(19) United States
(12) Patent Application Publication
(10) Pub. No.: US 2006/0004197 A1
(43) Pub. Date: Jan. 5, 2006

(54) Sulfonamide-based compounds as protein tyrosine kinase inhibitors

(76) Inventors: Thomas Thrash, Houston, TX (US); Michael S. Lawless, St. Charles, MO (US); Julian Smith, Whitestone (GB); Richard Foster, Cornwall (GB); Qian Liu, Ballwin, MO (US); Raymond J.A. Budde, Bellaire, TX (US)

Correspondence Address: Michael R. Hull;MARSHALL, GERSTEIN & BORUN, Sears Tower, Suite 6300, 233 S. Wacker Drive, Chicago, IL, 60606-6357, US

(21) Appl. No.: 10/884,113

(22) Filed: Jul. 2, 2004

(51) Int. Cl. C07D 279/12; C07D 213/53; C07D 241/04; C07C 311/31

(52) U.S. Cl. 544/59; 564/76; 544/392; 546/331

(57) ABSTRACT

Various sulfonamide-based compounds are able to selectively inhibit the Src family of tyrosine kinases. These compounds are useful in the treatment of various diseases including hyperproliferative diseases, hematologic diseases, osteoporosis, neurological diseases, autoimmune diseases, allergic/immunological diseases, or viral infections.

### BACKGROUND

1. Technical Field

Novel sulfonamide compounds are disclosed which are useful for the treatment of diseases related to increased protein tyrosine kinase activity. Methods of synthesis of these compounds and methods of treatment employing these compounds are also disclosed. The novel compounds include mono-sulfonamides and bis-sulfonamides capable of inhibiting the protein tyrosine kinases (PTKs).

2. Background of the Related Art

Within the Src family of PTKs, Src is associated with cellular membranes and is involved in signal transduction and growth regulation pathways (Sridhar and Cooper, 2000, Frame, 2002). Src propagates cellular signals by transferring the gamma phosphate of ATP to the side chain of tyrosine residues on substrate proteins. Alterations in the phosphorylation of Src substrates are key events in cellular signaling. Most normal cells contain very low levels and activity of Src (Barnekow, 1989) and the Src enzyme is not required for the establishment or maintenance of cell viability (Soriano, et al., 1991).

However, excessive Src activity is associated with various cancers, and therefore Src is a drug target in oncology (Cartwright et al., 1990). For example, Src activity is greatly increased in breast cancer (Partanen, 1994); stomach cancer (Takeshima et al., 1991); colon cancer (Termuhlen et al., 1993); hairy cell leukemia and a subgroup of B-cell lymphomas (Lynch et al., 1993); low grade human bladder carcinoma (Fanning et al., 1992); neuroblastoma (Bjelfman et al., 1990); ovarian cancer (Wiener et al., 1999); and non-small cell lung carcinoma (Budde et al., 1994). In the case of colon cancer, Src is activated more frequently than Ras or p53 (Jessup et al., 1993). Src undergoes two distinct activations corresponding with malignant transformation of colonocytes (Cartwright et al., 1990) and tumor progression (Termuhlen et al., 1993).

Antisense to Src inhibits growth of human monoblastoid leukemia cells (Waki et al., 1994), K562 human leukemia cells (Kitanaka et al., 1994) and HT-29 human colon cancer cells (Staley et al., 1997). Src activity has been reduced in a human ovarian cancer cell line (SKOv-3) by antisense technology. The reduced Src activity in SKOv-3 is associated with altered cellular morphology, reduced anchorage-independent growth, diminished tumor growth and reduced vascular endothelial growth factor mRNA expression in vitro (Wiener et al., 1999).

Inhibition of Src would have the effect of interrupting the signal transduction pathways in which it participates and would thereby reduce the rate of growth of cancer cells.

Src inhibitors are currently being studied for use in the treatment of hematologic and solid tumors, inflammatory and autoimmune diseases (Sinha et al., 1999). Src inhibitors have potential for treatment of osteoporosis, a condition in which bone resorption is increased resulting in weakening of bone. It was shown that mice depleted of the Src gene developed osteopetrosis (Soriano et al., 1991) and that Src is involved with bone resorption (20).

Potential sites for targeting inhibitors of Src family PTKs are the SH2 and SH3 domains (Park et al., 2003), the phosphoryl transfer site (SH1 domain), i.e., the active site or other unknown sites on the enzyme. Compounds binding to SH2 and SH3 domains would block the protein-protein interactions and the recruitment of other signal transduction proteins mediated by these domains. Active-site directed inhibitors could be targeted to the ATP binding site, the protein substrate binding site, or both (bisubstrate analogues).

### SUMMARY OF THE DISCLOSURE

In satisfaction of the aforenoted needs, disclosed herein are a number of small-molecule sulfonamide PTK inhibitors that are suitable to act as pharmaceuticals. The inhibitors disclosed herein may be targeted to the phosphoryl transfer site (SH1 domain), i.e., the active site. Active-site directed inhibitors can be targeted to the ATP binding site, the protein substrate binding site, or both (bisubstrate analogues). While the disclosed sulfonamide compounds serve as inhibitors for the Src family of PTKs, it will be understood that the disclosed compounds may very well serve as inhibitors to additional families of PTKs or other protein kinases as well.

The disclosed compounds are selected from the following general formulas: 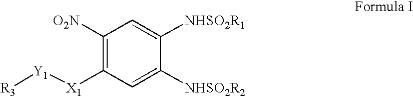
wherein R₁ is p-(C₆H₄)CH₃ or CH₃;
wherein R₂ is p-(C₆H₄)CH₃ or CH₃;
wherein R₃ is F, Cl, p-(C₆H₄)OCH₃, o-(C₆H₄)OCH₃, m-(C₆H₄)OCH₃, p-(C₆H₄)OH, p-(C₆H₄)Cl, o-(C₆H₄)Cl, m-(C₆H₄)Cl, p-(C₆H₄)F, p-(C₆H₄)CH₃, o-(C₆H₄)CH₃, M-(C₆H₄)CH₃, 3,5-(C₆H₃)(CH₃)₂, 2,6-(C₆H₃)(CH₃)₂, o-(C₆H₄)[CH(CH₃)₂], C₆H₅, 2-furyl, morpholin-4-yl, n-Me-piperazin-1-yl, thiomorpholin-4-yl, 3-pyridyl, 2-pyridyl, cyclohexyl, cyclohexyl-1-ol, or 5-Me-pyrazol-4-yl;
wherein X₁ is S, NH or O; and
wherein Y₁ is (CH₂)ₙ wherein n ranges from 1 to 3; 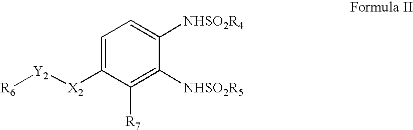
wherein R₄ is p-(C₆H₄)CH₃, p-(C₆H₄)(CH₂CH₃), p-(C₆H₄)[CH(CH₃)₂], p-(C₆H₄)Cl, p-(C₆H₄)F, p-(C₆H₄)OC₆H₅, m-(C₆H₄)NO₂, CH₂(C₆H₅), 1-naphthyl, 2-naphthyl, p-(C₆H₄)NH(C═O)CH₃, p-(C₆H₄)OCH₃, (CH₂)₃CH₃, 3,4-(C₆H₃)(OCH₃)₂, C₆H₅, p-(C₆H₄)[C(CH₃)]₃, 2,4,6-(C₆H₂)(CH₃)₃, p-(C₆H₄)NO₂, CH₃, 4-methyl-2-acetamidothiazol-5-yl, 3,5-dimethylisoxazol-4-yl, 1-methylimidazol-4-yl, 5-Br-6-Cl-pyrid-3-yl, 7-Cl-benzo[1,2,5]oxadiazol-4-yl, 5-[3-(isoxazolyl)]thien-2-yl, 1,2,5-trimethyl-3-carbomethoxypyrrol-4-yl, p-(C₆H₄)CH₂CH₂CH₂CH₃, 2-(1-naphthyl)ethyl, p-(C₆H₄)SO₂CH₃, m-(C₆H₄)OCH₃, 5-bromothien-2-yl, or isoquinolin-5-yl;
wherein R₅ is p-(C₆H₄)CH₃, p-(C₆H₄)(CH₂CH₃), p-(C₆H₄)[CH(CH₃)₂], p-(C₆H₄)Cl, p-(C₆H₄)F, p-(C₆H₄)OC₆H₅, m-(C₆H₄)NO₂, CH₂(C₆H₅), 1-naphthyl, 2-naphthyl, p-(C₆H₄)NH(C═O)CH₃, p-(C₆H₄)OCH₃, (CH₂)₃CH₃, 3,4-(C₆H₃)(OCH₃)₂, C₆H₅, p-(C₆H₄)[C(CH₃)]₃, 2,4,6-(C₆H₂)(CH₃)₃, p-(C₆H₄)NO₂, CH₃, 3,5-dimethylisoxazol-4-yl, 1-methylimidazol-4-yl, 1,2,5-trimethyl-3-carbomethoxypyrrol-4-yl, 2-(1-naphthyl)ethyl, p-(C₆H₄)SO₂CH₃, 5-bromothien-2-yl, or 2-methoxy-4-methylphenyl;
wherein R₆ is F, Cl, p-(C₆H₄)OCH₃, (CH₂)₄CH₂OH, p-(C₆H₄)F, 2-naphthyl, CH₃, p-(C₆H₄)Cl, m-(C₆H₄)CO₂H, m-(C₆H₄)CH₂CO₂H, p-(C₆H₄)CO₂H, p-(C₆H₄)CH₂CO₂H, CH₂CH₂CH₂OH, allyl, (CH₂)₇CH₂OH, (CH₂)₇CH₂C(═O)CH₃, (CH₂)₃CH₃, m-(C₆H₄)Cl, o-(C₆H₄)Cl, cyclohexyl, pyrazol-1-yl, benzimidazol-1-yl, N(CH₃)₂, imidazol-1-yl, N-(4-toluenesulfonyl)piperazin-1-yl, morpholin-4-yl, p-CH₂CH₂(C₆H₄)OCH₃, C(CH₃)₃, 3-pyridyl, C₆H₅, CH₂C(CH₃)₂CH₂OH, CH₂(naphth-1-yl), CH₂C₆H₅, 2-thienyl, 3,5-dimethylisoxazol-4-yl, or OH;
wherein R₇ is H, CH₂CHCH₂ or OCH₃;
wherein X₂ is O, S, H, C(═O), NH, CH₂, C(═NOCH₂C₆H₅), C(═NOH), or C(═NOCH₃); and
wherein Y₂ is CH₂, NH, C(═O), or SO₂; 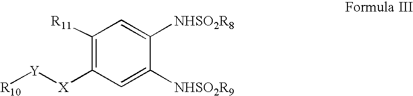
wherein R₈ is p-(C₆H₄)CH₃;
wherein R₉ is p-(C₆H₄)CH₃;
wherein R₁₀ is p-(C₆H₄)Cl, p-(C₆H₄)OCH₃, Br, or o-(C₆H₄)Cl,
wherein R₁₁ is NH₂, NHC(═O)CH₃, CO₂CH₃, CO₂H, CH₃, Br, CF₃, or F;
wherein X₃ is S, O, or SO₂; and
wherein Y₃ is CH₂; 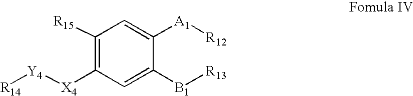
wherein R₁₂ is p-(C₆H₄)CH₃, OCH₃, H, NH₂, 2-cyanopyrid-5-yl, 2-trifluoromethylpyrid-5-yl, p-(C₆H₄)CN, p-(C₆H₄)NHC(═O)CH₃, benzofuran-2-yl, quinolin-2-yl, 5-methyl-pyrazin-2-yl, CN, CO₂H, C₆H₅, C(═O)NH₂, CH₂NH₂, CH₃, 7-chloro-benzo[1,2,5]oxadiazol-4-yl, and p-(C₆H₄)CH₂CH₃,
wherein R₁₃ is p-(C₆H₄)CH₃, OP(═O)(OCH₂CH₃)₂, OP(═O)(OH)₂, p-(C₆H₄)CH₃, NH₂, OH, OCH₃, CH₂CH₂OH, CH₂CH₂CH₂OH, rac-CH₂CH(OH)CH₂OH, CH₂CO₂H, CO₂CH₃, CO₂H, H, CH₂NH₂, quinolin-2-yl, 6-methylnaphth-2-yl, or CH₂CH₂CH₃;
wherein R₁₄ is p-(C₆H₄)OCH₃, p-(C₆H₄)Cl, C₆H₅, morpholin-4-yl, cyclohexyl, naphth-1-yl, o-(C₆H₄)Cl, or CH₃;
wherein R₁₅ is H, NO₂, or CF₃;
wherein X₄ O, CH₂, S, NH, or SO₂;
wherein Y₄ is O, C(═O), or CH₂;
wherein A is NHSO₂, O—SO₂, NHC(═O), N(CH₃)SO₂, NHCH₂, CH₂NHSO₂, NHC(═O)CH₂, CH₂NHC(═O), or SO₂NH;
wherein B is O—SO₂, NHC(═O), N(CH₃)SO₂, O, NHSO₂, CH₂NHSO₂, N[C(═O)C₆H₅)]SO₂, CH₂NHC(═O), E/Z N═C, NHC(═O), NH₂CH₂, or NHC(═O)NHSO₂; 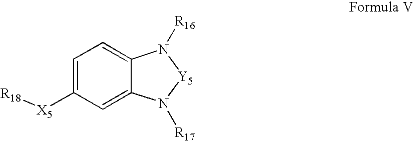
wherein R₁₆ is p-(C₆H₄)CH₃ or H;
wherein R₁₇ is p-(C₆H₄)CH₃ or H;
wherein R₁₈ is p-(C₆H₄)OCH₃;
wherein X₅ is O; and
wherein Y₅ is C(═O) or SO₂.

In an embodiment, the PTK inhibitor compound is selected from the group consisting of:
N-[4-fluoro-5-nitro-2-(4-toluenesulfonylamino)-phenyl]-4-toluenesulfonamide;
N-[4-(4-methoxy-phenylsulfanyl)-5-nitro-2-(4-toluenesulfonylamino)-phenyl]-4-toluenesulfonamide;
N-[4-(2-methoxy-phenylsulfanyl)-5-nitro-2-(4-toluenesulfonylamino)-phenyl]-4-toluenesulfonamide;
N-[4-(3-methoxy-phenylsulfanyl)-5-nitro-2-(4-toluenesulfonylamino)-phenyl]-4-toluenesulfonamide;
N-[4-(4-hydroxy-phenylsulfanyl)-5-nitro-2-(4-toluenesulfonylamino)-phenyl]-4-toluenesulfonamide;
N-[4-(4-chloro-phenylsulfanyl)-5-nitro-2-(4-toluenesulfonylamino)-phenyl]-4-toluenesulfonamide;
N-[4-(2-chloro-phenylsulfanyl)-5-nitro-2-(4-toluenesulfonylamino)-phenyl]-4-toluenesulfonamide;
N-[4-(3-chloro-phenylsulfanyl)-5-nitro-2-(4-toluenesulfonylamino)-phenyl]-4-toluenesulfonamide;
N-[4-(4-fluoro-phenylsulfanyl)-5-nitro-2-(4-toluenesulfonylamino)-phenyl]-4-toluenesulfonamide;
N-[5-nitro-2-(4-toluenesulfonylamino)-4-(p-tolylsulfanyl)-phenyl]-4-toluenesulfonamide;
N-[5-nitro-2-(4-toluenesulfonylamino)-4-(o-tolylsulfanyl)-phenyl]-4-toluenesulfonamide;
N-[5-nitro-2-(4-toluenesulfonylamino)-4-(m-tolylsulfanyl)-phenyl]-4-toluenesulfonamide;
N-[4-(2,4-dimethyl-phenylsulfanyl)-5-nitro-2-(4-toluenesulfonylamino)-phenyl]-4-toluenesulfonamide;
N-[4-(2,6-dimethyl-phenylsulfanyl)-5-nitro-2-(4-toluenesulfonylamino)-phenyl]-4-toluenesulfonamide;
N-[4-(2-isopropyl-phenylsulfanyl)-5-nitro-2-(4-toluenesulfonylamino)-phenyl]-4-toluenesulfonamide;
N-[5-nitro-4-phenylsulfanyl-2-(4-toluenesulfonylamino)-phenyl]-4-toluenesulfonamide;
N-[4-(furan-2-ylmethylsulfanyl)-5-nitro-2-(4-toluenesulfonylamino)-phenyl]-4-toluenesulfonamide;
N-[4-(4-chloro-benzylsulfanyl)-5-nitro-2-(4-toluenesulfonylamino)-phenyl]-4-toluenesulfonamide;
N-[4-(2-chloro-benzylsulfanyl)-5-nitro-2-(4-toluenesulfonylamino)-phenyl]-4-toluenesulfonamide;
N-[4-(4-methoxy-benzylsulfanyl)-5-nitro-2-(4-toluenesulfonylamino)-phenyl]-4-toluenesulfonamide;
N-[4-benzylsulfanyl-5-nitro-2-(4-toluenesulfonylamino)-phenyl]-4-toluenesulfonamide;
N-[5-(4-chloro-phenylsulfanyl)-2-methanesulfonylamino-4-nitro-phenyl]-methanesulfonamide;
N-[4-chloro-5-nitro-2-(4-toluenesulfonylamino)-phenyl]-4-toluenesulfonamide;
N-[5-nitro-4-phenyl-2-(4-toluenesulfonylamino)-phenyl]-4-toluenesulfonamide;
N-[4-(morpholin-4-yl)-5-nitro-2-(4-toluenesulfonylamino)-phenyl]-4-toluenesulfonamide;
N-[4-(4-methyl-piperazin-1-yl)-5-nitro-2-(4-toluenesulfonylamino)-phenyl]-4-toluenesulfonamide;
N-[5-nitro-4-(thiomorpholin-4-yl)-2-(4-toluenesulfonylamino)-phenyl]-4-toluenesulfonamide;
N-[5-nitro-4-[(pyridin-3-ylmethyl)-amino]-2-(4-toluenesulfonylamino)-phenyl]-4-toluenesulfonamide;
N-[5-nitro-4-[(pyridin-2-ylmethyl)-amino]-2-(4-toluenesulfonylamino)-phenyl]-4-toluenesulfonamide;
N-[4-(4-methoxy-benzylamino)-5-nitro-2-(4-toluenesulfonylamino)-phenyl]-4-toluenesulfonamide;
N-[4-(2-chloro-benzylamino)-5-nitro-2-(4-toluenesulfonylamino)-phenyl]-4-toluenesulfonamide;
N-[4-(cyclohexylmethyl-amino)-5-nitro-2-(4-toluenesulfonylamino)-phenyl]-4-toluenesulfonamide;
N-[4-(1-hydroxy-cyclohexylmethyl-amino)-5-nitro-2-(4-toluenesulfonylamino)-phenyl]-4-toluenesulfonamide;
N-[4-cyclohexylamino-5-nitro-2-(4-toluenesulfonylamino)-phenyl]-4-toluenesulfonamide;
N-[4-[3-(5-methyl-1H-pyrazol-4-yl)-propylamino]-5-nitro-2-(4-toluenesulfonylamino)-phenyl]-4-toluenesulfonamide;
N-[4-fluoro-2-(4-toluenesulfonylamino)-phenyl]-4-toluenesulfonamide;
N-[4-chloro-2-(4-toluenesulfonylamino)-phenyl]-4-toluenesulfonamide;
4-ethyl-N-[2-(4-ethyl-benzenesulfonylamino)-4-(4-methoxy-phenoxy)-phenyl]-benzenesulfonamide;
4-isopropyl-N-[2-(4-isopropyl-benzenesulfonylamino)-4-(4-methoxy-phenoxy)-phenyl]-benzenesulfonamide;
4-chloro-N-[2-(4-chloro-benzenesulfonylamino)-4-(4-methoxy-phenoxy)-phenyl]-benzenesulfonamide;
4-fluoro-N-[2-(4-fluoro-benzenesulfonylamino)-4-(4-methoxy-phenoxy)-phenyl]-benzenesulfonamide;
N-[4-(4-methoxy-phenoxy)-2-(4-phenoxy-benzenesulfonylamino)-phenyl]-4-phenoxy-benzenesulfonamide;
N-[4-(4-methoxy-phenoxy)-2-(3-nitro-benzenesulfonylamino)-phenyl]-3-nitro-benzenesulfonamide;
N-[4-(4-methoxy-phenoxy)-2-phenylmethanesulfonylamino-phenyl]-C-phenyl-methanesulfonamide;
naphthalene-1-sulfonic acid [4-(4-methoxy-phenoxy)-2-(naphthalen-1-yl-sulfonylamino)-phenyl]-amide;
naphthalene-2-sulfonic acid [4-(4-methoxy-phenoxy)-2-(naphthalen-2-yl-sulfonylamino)-phenyl]-amide;
N-[2-(4-acetamido-benzenesulfonylamino)-4-(4-methoxy-phenoxy)-phenyl]-4-acetamido-benzenesulfonamide;
N-[2-(4-methoxy-benzenesulfonylamino)-4-(4-methoxy-phenoxy)-phenyl]-4-methoxy-benzenesulfonamide;
butane-1-sulfonic acid [2-(butane-1-sulfonylamino)-4-(4-methoxy-phenoxy)-phenyl]-amide;
N-[2-(3,4-dimethoxy-benzenesulfonylamino)-4-(4-methoxy-phenoxy)-phenyl]-3,4-dimethoxy-benzenesulfonamide;
N-[2-benzenesulfonylamino-4-(4-methoxy-phenoxy)-phenyl]-benzenesulfonamide;
N-[2-(4-t-butyl-benzenesulfonylamino)-4-(4-methoxy-phenoxy)-phenyl]-4-t-butyl-benzenesulfonamide;
N-[4-(4-methoxy-phenoxy)-2-(4-toluenesulfonylamino)-phenyl]-4-toluenesulfonamide;
N-[4-(4-methoxy-phenoxy)-2-(2,4,6-trimethylbenzenesulfonylamino)-phenyl]-2,4,6-trimethylbenzenesulfonamide;
N-[4-(4-methoxy-phenoxy)-2-(4-nitrobenzenesulfonylamino)-phenyl]-4-nitrobenzenesulfonamide;
N-[4-(5-hydroxy-pentyloxy)-2-(4-toluenesulfonylamino)-phenyl]-4-toluenesulfonamide;
N-[4-(4-fluoro-phenoxy)-2-(4-toluenesulfonylamino)-phenyl]-4-toluenesulfonamide;
N-[4-(naphthalene-2-yloxy)-2-(4-toluenesulfonylamino)-phenyl]-4-toluenesulfonamide;
N-[4-methoxy-2-(4-toluenesulfonylamino)-phenyl]-4-toluenesulfonamide
N-[4-(4-chloro-phenoxy)-2-(4-toluenesulfonylamino)-phenyl]-4-toluenesulfonamide;
N-[4-(3-carboxy-phenoxy)-2-(4-toluenesulfonylamino)-phenyl]-4-toluenesulfonamide;
{3-[3,4-bis(4-toluenesulfonylamino)-phenoxy]-phenyl}-acetic acid;
N-[4-(3-carboxy-phenoxy)-2-(4-toluenesulfonylamino)-phenyl]-4-toluenesulfonamide;
{4-[3,4-bis(4-toluenesulfonylamino)-phenoxy]-phenyl}-acetic acid;
N-[4-(3-hydroxy-propoxy)-2-(4-toluenesulfonylamino)-phenyl]-4-toluenesulfonamide;
N-[4-allyloxy-2-(4-toluenesulfonylamino)-phenyl]-4-toluenesulfonamide;
N-[4-(8-hydroxy-octyloxy)-2-(4-toluenesulfonylamino)-phenyl]-4-toluenesulfonamide;
5-[3,4-bis-(4-toluenesulfonylamino)-phenoxy]-pentyl acetate;
N-[4-(4-chloro-phenylsulfanyl)-2-(4-toluenesulfonylamino)-phenyl]-4-toluenesulfonamide;
N-[4-(4-methoxy-phenylsulfanyl)-2-(4-toluenesulfonylamino)-phenyl]-4-toluenesulfonamide;
N-[4-butylsulfanyl-2-(4-toluenesulfonylamino)-phenyl]-4-toluenesulfonamide;
N-[3-allyl-4-(4-methoxy-benzyloxy)-2-(4-toluenesulfonylamino)-phenyl]-4-toluenesulfonamide;
N-[4-(4-chloro-benzyloxy)-2-(4-toluenesulfonylamino)-phenyl]-4-toluenesulfonamide;
N-[4-(3-chloro-benzyloxy)-2-(4-toluenesulfonylamino)-phenyl]-4-toluenesulfonamide;
N-[4-(2-chloro-benzyloxy)-2-(4-toluenesulfonylamino)-phenyl]-4-toluenesulfonamide;
N-[4-(4-methoxy-benzyloxy)-2-(4-toluenesulfonylamino)-phenyl]-4-toluenesulfonamide;
N-[4-cyclohexylmethoxy-2-(4-toluenesulfonylamino)-phenyl]-4-toluenesulfonamide;
N-[4-(2-chloro-benzylsulfanyl)-2-(4-toluenesulfonylamino)-phenyl]-4-toluenesulfonamide;
N-[4-(pyrazol-1-yl)-2-(4-toluenesulfonylamino)-phenyl]-4-toluenesulfonamide;
N-[4-(benzimidazol-1-yl)-2-(4-toluenesulfonylamino)-phenyl]-4-toluenesulfonamide;
N-[4-dimethylamino-2-(4-toluenesulfonylamino)-phenyl]-4-toluenesulfonamide;
N-[4-(imidazol-1-yl)-2-(4-toluenesulfonylamino)-phenyl]-4-toluenesulfonamide;
N-{2-(4-toluenesulfonylamino)-4-[4-(toluene-4-sulfonyl)-piperazin-1-yl]-phenyl}-4-toluenesulfonamide;
N-[4-(morpholin-4-yl)-2-(4-toluenesulfonylamino)-phenyl]-4-toluenesulfonamide;
N-[4-(4-chloro-phenyl)-2-(4-toluenesulfonylamino)-phenyl]-4-toluenesulfonamide;
N-[3-methoxy-2-(4-toluenesulfonylamino)-phenyl]-4-toluenesulfonamide;
N-[2-(4-methoxy-phenyl)-ethyl]-3,4-bis-(4-toluenesulfonylamino)-benzamide;
N-t-butyl-3,4-bis-(4-toluenesulfonylamino)-benzamide;
N-pyridin-3-yl-3,4-bis-(4-toluenesulfonylamino)-benzamide;
N-phenyl-3,4-bis-(4-toluenesulfonylamino)-benzamide;
N-(3-hydroxy-2,2-dimethyl-propyl)-3,4-bis-(4-toluenesulfonylamino)-benzamide;
N-naphthalen-1-ylmethyl-3,4-bis-(4-toluenesulfonylamino)-benzamide;
2-phenyl-N-[3,4-bis-(4-toluenesulfonylamino)-phenyl]-acetamide;
N-[3,4-bis-(4-toluenesulfonylamino)-phenyl]-thiophene-2-sulfonamide;
3,5-dimethyl-N-[3,4-bis-(4-toluenesulfonylamino)-phenyl]-isoxazole-4-sulfonamide;
3,4-bis-(4-toluenesulfonylamino)-benzoic acid;
N-[4-hydroxymethyl-2-(4-toluenesulfonylamino-phenyl]-4-toluenesulfonamide;
N-[2-methanesulfonylamino-4-(4-methoxy-phenoxy)-phenyl]-4-toluenesulfonamide;
3,5-dimethyl-N-[5-(4-methoxy-phenoxy)-2-(4-toluenesulfonylamino)-phenyl]-isoxazole-4-sulfonamide;
N-[5-(4-methoxy-phenoxy)-2-(4-toluenesulfonylamino)-phenyl]-1-methyl-1H-imidazole-4-sulfonamide;
N-[2-methanesulfonylamino-5-(4-methoxy-phenoxy)-phenyl]-4-toluenesulfonamide;
N-{5-[4-(4-methoxy-phenoxy)-2-(4-toluenesulfonylamino)-phenylsulfamoyl]-4-methyl-thiazol-2-yl}-acetamide;
3,5-dimethyl-N-[4-(4-methoxy-phenoxy)-2-(4-toluenesulfonylamino)-phenyl]-isoxazole-4-sulfonamide;
N-[4-(4-methoxy-phenoxy)-2-(4-toluenesulfonylamino)-phenyl]-1-methyl-1H-imidazole-4-sulfonamide;
5-bromo-6-chloro-N-[4-(4-methoxy-phenoxy)-2-(4-toluenesulfonylamino)-phenyl]-pyridine-3-sulfonamide;
7-chloro-N-[4-(4-methoxy-phenoxy)-2-(4-toluenesulfonylamino)-phenyl]-benzo[1,2,5]oxadiazole-4-sulfonamide;
5-isoxazol-3-yl-N-[4-(4-methoxy-phenoxy)-2-(4-toluenesulfonylamino)-phenyl]-thiophene-2-sulfonamide;
methyl 4-[4-(4-methoxy-phenoxy)-2-(4-toluenesulfonylamino)-phenylsulfamoyl]-1,2,5-trimethyl-1H-pyrrole-3-carboxylate;
4-butyl-N-[4-(4-methoxy-phenoxy)-2-(4-toluenesulfonylamino)-phenyl]-benzenesulfonamide;
N-[5-(4-methoxy-phenoxy)-2-(2-naphthalen-1-yl-ethanesulfonylamino)-phenyl]-4-toluenesulfonamide;
4-methanesulfonyl-N-[4-(4-methoxy-phenoxy)-2-(4-toluenesulfonylamino)-phenyl]-benzenesulfonamide;
3-methoxy-N-[4-(4-methoxy-phenoxy)-2-(4-toluenesulfonylamino)-phenyl]-benzenesulfonamide;
N-[4-(4-methoxy-phenoxy)-2-(4-toluenesulfonylamino)-phenyl]-5-bromo-thiophene-2-sulfonamide;
N-[4-(4-methoxy-phenoxy)-2-(4-toluenesulfonylamino)-phenyl]-isoquinoline-5-sulfonamide;
methyl 4-[5-(4-methoxy-phenoxy)-2-(4-toluenesulfonylamino)-phenylsulfamoyl]-1,2,5-trimethyl-1H-pyrrole-3-carboxylate;
N-[4-(4-methoxy-phenoxy)-2-(2-naphthalen-1-yl-ethanesulfonylamino)-phenyl]-4-toluenesulfonamide;
4-methanesulfonyl-N-[5-(4-methoxy-phenoxy)-2-(4-toluenesulfonylamino)-phenyl]-benzenesulfonamide;
5-bromo-N-[5-(4-methoxy-phenoxy)-2-(4-toluenesulfonylamino)-phenyl]-thiophene-2-sulfonamide;
2-methoxy-N-[5-(4-methoxy-phenoxy)-2-(4-toluenesulfonylamino)-phenyl]-4-methyl-benzenesulfonamide;
N-[4-(benzyloxyimino-phenyl-methyl)-2-(4-toluenesulfonylamino)-phenyl]-4-toluenesulfonamide;
N-[4-(hydroxyimino-phenyl-methyl)-2-(4-toluenesulfonylamino)-phenyl]-4-toluenesulfonamide;
N-[4-(methoxyimino-phenyl-methyl)-2-(4-toluenesulfonylamino)-phenyl]-4-toluenesulfonamide;
N-[5-(2-chloro-benzylsulfanyl)-2-methanesulfonylamino-phenyl]-4-toluenesulfonamide;
N-[4-(2-chloro-benzylsulfanyl)-2-(4-toluenesulfonylamino)-phenyl]-4-toluenesulfonamide;
N-[4-(2-chloro-benzylsulfanyl)-2-methanesulfonylamino-phenyl]-4-toluenesulfonamide;
N-[5-amino-4-(4-chloro-phenylsulfanyl)-2-(4-toluenesulfonylamino)-phenyl]-4-toluenesulfonamide;
N-[2-(4-chloro-phenylsulfanyl)-4,5-bis-(4-toluenesulfonylamino)-phenyl]-acetamide;
methyl 2-(4-methoxy-phenoxy)-4,5-bis-(4-toluenesulfonylamino)-benzoate;
2-(4-methoxy-phenoxy)-4,5-bis-(4-toluenesulfonylamino)-benzoic acid;
N-[4-(4-methoxy-phenoxy)-5-methyl-2-(4-toluenesulfonylamino)-phenyl]-4-toluenesulfonamide;
N-[4,5-dibromo-2-(4-toluenesulfonylamino)-phenyl]-4-toluenesulfonamide;
N-[4-(4-methoxy-phenoxy)-2-(4-toluenesulfonylamino)-5-trifluoromethyl-phenyl]-4-toluenesulfonamide;
N-[4-(2-chloro-benzylsulfanyl)-5-fluoro-2-(4-toluenesulfonylamino)-phenyl]-4-toluenesulfonamide;
N-[4-(2-chloro-phenylmethanesulfonyl)-5-fluoro-2-(4-toluenesulfonylamino)-phenyl]-4-toluenesulfonamide;
diethyl-5-(4-methoxy-phenoxy)-2-(4-toluenesulfonylamino)-phenyl phosphate;
[5-(4-methoxy-phenoxy)-2-(4-toluenesulfonylamino)-phenyl]-monophosphate;
4-(4-methoxy-phenoxymethyl)-2-(4-toluenesulfonyloxy)-phenyl 4-toluenesulfonate;
N-[4-(4-chloro-phenylsulfanyl)-5-nitro-2-(4-toluamido)-phenyl]-4-toluamide;
N-[4-(4-chloro-phenylsulfanyl)-2-(4-toluoylamino)-phenyl]-4-toluamide;
N-[4-(4-chloro-phenylsulfanyl)-5-nitro-2-(4-toluenesulfonyl-methyl-amino)-phenyl]-N-methyl-4-toluenesulfonamide;
N-[2-amino-4-(4-chloro-phenylsulfanyl)-5-nitro-phenyl]-4-toluenesulfonamide;
N-[2-amino-4-(4-chloro-phenylsulfanyl)-5-nitro-phenyl]-4-toluamide;
N-[2-hydroxy-4-(4-methoxy-phenoxy)-phenyl]-4-toluenesulfonamide;
N-[2-methoxy-4-(4-methoxy-phenoxy)-phenyl]-4-toluenesulfonamide;
N-[2-methoxy-4-(4-methoxy-phenoxy)-phenyl]-N-methyl-4-toluenesulfonamide;
N-[2-(2-hydroxy-ethoxy)-4-(4-methoxy-phenoxy)-phenyl]-4-toluenesulfonamide;
N-[2-(3-hydroxy-propoxy)-4-(4-methoxy-phenoxy)-phenyl]-4-toluenesulfonamide;
N-[2-(2,3-dihydroxy-propoxy)-4-(4-methoxy-phenoxy)-phenyl]-4-toluenesulfonamide;
[5-(4-methoxy-phenoxy)-2-(4-toluenesulfonylamino)-phenoxy]-acetic acid;
N-[2-methoxy-5-(4-methoxy-phenoxymethyl)-phenyl]-4-toluenesulfonamide;
methyl 5-(4-methoxy-phenoxy)-2-(4-toluenesulfonylamino)-benzoate;
5-(4-methoxy-phenoxy)-2-(4-toluenesulfonylamino)-benzoic acid;
N-[4-(4-chloro-phenylsulfanyl)-3-nitro-phenyl]-4-toluenesulfonamide;
N-[3-(4-chloro-phenylsulfanyl)-4-nitro-phenyl]-4-toluenesulfonamide;
N-[2-amino-5-(4-methoxy-phenoxy)-phenyl]-4-toluenesulfonamide;
6-cyano-N-[4-(4-methoxy-phenoxy)-2-(4-toluenesulfonylamino)-phenyl]-nicotinamide;
N-[4-(4-methoxy-phenoxy)-2-(4-toluenesulfonylamino)-phenyl]-6-trifluoromethyl-nicotinamide;
N-[2-(4-cyano-benzylamino)-5-(4-methoxy-phenoxy)-phenyl]-4-toluenesulfonamide;
N-(4-{[4-(4-methoxy-phenoxy)-2-(4-toluenesulfonylamino)-phenylamino]-methyl}-phenyl)-acetamide;
N-[2-[(benzofuran-2-ylmethyl)-amino]-5-(4-methoxy-phenoxy)-phenyl]-4-toluenesulfonamide;
N-{5-(4-methoxy-phenoxy)-2-[(quinolin-2-ylmethyl)-amino]-phenyl}-4-toluenesulfonamide;
N-[5-(4-methoxy-phenoxy)-2-(4-toluenesulfonylamino)-phenyl]-5-methyl-pyrazine-2-carboxamide;
N-[2-amino-5-(4-methoxy-phenoxy)-benzyl]-4-toluenesulfonamide;
N-[2-cyano-4-(4-methoxy-phenoxy)-phenyl]-4-toluenesulfonamide;
5-(4-methoxy-phenoxy)-2-(4-toluenesulfonylamino)-benzamide;
N-[2-amino-4-(4-methoxy-phenoxy)-phenyl]-4-toluenesulfonamide;
N-[4-(4-methoxy-phenoxy)-2-(4-toluenesulfonylamino-methyl)-phenyl]-4-toluenesulfonamide;
N-[2-aminomethyl-4-(4-methoxy-phenoxy)-phenyl]-4-toluenesulfonamide;
N-[3-[benzoyl-(4-toluenesulfonyl)-amino]-4-(4-toluenesulfonylamino)-phenyl]-benzamide;
N-[5-(4-methoxy-phenoxy)-2-(4-toluenesulfonylamino)-benzyl]-4-toluamide;
N-[5-(4-methoxy-phenoxy)-2-(4-toluenesulfonylamino)-phenyl]-4-toluamide;
N-[4-(4-methoxy-phenoxy)-2-(4-toluenesulfonylamino)-phenyl]-4-toluamide;
N-[4-(4-methoxy-phenoxy)-2-(4-toluenesulfonylamino)-phenyl]-2-phenyl-acetamide;
N-[2-amino-5-(4-methoxy-phenoxy)-phenyl]-N-methyl-4-toluenesulfonamide;
N-{4-(4-methoxy-phenoxy)-2-[(quinolin-2-ylmethylene)-amino]-phenyl}-4-toluenesulfonamide;
4-(4-methoxy-phenoxymethyl)-2-(4-toluenesulfonylamino)-benzamide;
N-[2-aminomethyl-5-(4-methoxy-phenoxymethyl)-phenyl]-4-toluenesulfonamide;
N-[2-(methanesulfonylamino-methyl)-5-(4-methoxy-phenoxymethyl)-phenyl]-6-methyl-naphthalene-2-sulfonamide;
N-(4-morpholin-4-yl-5-nitro-2-(4-toluoylamino)-phenyl)-4-toluamide;
N-[5-(4-methoxy-phenoxy)-2-(4-toluenesulfonylamino)-phenyl]-butyramide;
N-[4-(cyclohexylmethyl-amino)-5-nitro-2-(4-toluoylamino)-phenyl]-4-toluamide;
N-{4-[(naphthalen-1-ylmethyl)-amino]-5-nitro-2-(4-toluoylamino)-phenyl}-4-toluamide;
N-[2-amino-4-(4-methoxy-phenoxy)-5-nitro-phenyl]-4-toluenesulfonamide;
N-[4-(4-methoxy-phenoxy)-2-(4-toluenesulfonyl-methyl-amino)-phenyl]-4-toluenesulfonamide;
N-[2-amino-4-(4-methoxy-phenoxy)-5-trifluoromethyl-phenyl]-4-toluenesulfonamide;
N-[4-(4-methoxy-phenoxymethyl)-2-(4-toluenesulfonylamino)-benzyl]-4-toluamide;
N-[2-amino-5-(4-methoxy-phenoxy)-4-trifluoromethyl-phenyl]-4-toluenesulfonamide;
N-[4-(4-methoxy-phenoxy)-2-(4-methyl-benzylamino)-phenyl]-4-toluenesulfonamide;
N-{4-(4-methoxy-phenoxy)-2-[3-(4-toluenesulfonyl)-ureido]-phenyl}-4-toluenesulfonamide;
N-[5-(4-methoxy-phenoxy)-2-(4-methyl-benzylamino)-phenyl]-4-toluenesulfonamide;
N-[2-amino-5-(2-chloro-benzylsulfanyl)-phenyl]-4-toluenesulfonamide;
N-[2-amino-4-(2-chloro-benzylsulfanyl)-5-nitro-phenyl]-7-chloro-benzo[1,2,5]oxadiazole-4-sulfonamide;
N-(4-ethyl-phenyl)-4-methoxy-2-(4-toluenesulfonylamino)-benzenesulfonamide;
N-[4-(2-chloro-phenylmethanesulfonyl)-2-(4-toluenesulfonylamino)-phenyl]-4-toluenesulfonamide;
6-(4-methoxy-phenoxy)-1-(4-toluenesulfonyl)-1,3-dihydro-benzoimidazol-2-one;
5-(4-methoxy-phenoxy)-1-(4-toluenesulfonyl)-1,3-dihydro-benzoimidazol-2-one;
5-(4-methoxy-phenoxy)-1,3-bis-(4-toluenesulfonyl)-1,3-dihydro-benzoimidazol-2-one;
5-(4-methoxy-phenoxy)-1,3-dihydro-benzo[1,2,5]thiadiazole 2,2-dioxide;
5-(4-methoxy-phenoxy)-1,3-dihydro-benzoimidazol-2-one;
5-(4-methoxy-phenoxy)-1-(4-toluenesulfonyl)-1,3-dihydro-benzo[1,2,5]thiadiazole 2,2-dioxide; and mixtures thereof.

A further embodiment is a pharmaceutical composition for the treatment of human and mammal diseases including but not limited to hyperproliferative diseases, hematologic diseases such as osteoporosis, neurological diseases such as Alzheimer's Disease, epilepsy or senility, autoimmune diseases, allergic/immunological diseases such as anaphylaxis, or viral infections which comprises a pharmaceutically acceptable carrier and a therapeutically effective amount of at least one cobalt complex disclosed herein or a pharmaceutically acceptable salt or hydrate thereof. The uses of the disclose PTK inhibiting sulfonamide compounds are not limited to the diseases listed herein.

Another embodiment is a method of synthesizing one or more of the sulfonamide compounds disclosed. Synthesis procedures are explained in detail below.

Another embodiment is a method of inhibiting PTKs by administering to a subject one or more sulfonamide compounds disclosed herein.

In a further embodiment, the step of the binding at least one of the disclosed sulfonamide compounds to protein tyrosine kinases may be included. In a further embodiment, the cell may be contacted with one or more of the disclosed sulfonamide compounds in order to alter cell morphology, migration, adhesion, cell cycle progression, secretion, differentiation, proliferation, anchorage-independent growth, vascular endothelial growth factor expression, microtubule binding by tau, viral infectivity, or bone reabsorption.

The protein tyrosine kinase may be Src, Fyn, Yes, Lyn, Lck, Blk, Hck, Fgr, or Yrk.

Another embodiment is a method of treating a PTK-related disease in a subject comprising the step of administering to the subject a pharmaceutically acceptable carrier and a therapeutically effective amount of one or more of the disclosed sulfonamide compounds.

In further embodiments, the administering may parenteral. In still further embodiments, the parenteral administration may be intravenous, intramuscular, subcutaneous, intraperitoneal, intraarterial, intrathecal or transdermal. In a further embodiment, the administering may be alimentary. In a further embodiment, the alimentary administration may be oral, rectal, sublingual, or buccal. In a further embodiment, the administration may be topical. In a further embodiment, the administration may be by inhalation. In a further embodiment, the administering may be combined with a second method of treatment.

Another embodiment is a method of preventing replication of a virus in an organism by administering to the organism infected with the virus one or more of the sulfonamide compounds disclosed herein. In a further embodiment, the virus may be a herpesvirus, papovavirus, hepadnavirus or retrovirus.

As used herein the specification, “a” or “an” may mean one or more. As used herein in the claim(s), when used in conjunction with the word “comprising,” the words “a” or “an” may mean one or more than one. As used herein “another” may mean at least a second or more.

Other features and advantages of the disclosed sulfonamide compounds, synthesis methods and treatment methods will become apparent from the following detailed description. It should be understood, however, that the detailed description and the specific examples, while indicating certain preferred embodiments, are given by way of illustration only, since various changes and modifications that fall within the spirit and scope of this disclosure will become apparent to those skilled in the art from this summary and the following detailed description.

### BRIEF DESCRIPTION OF THE DRAWINGS

The following drawings form part of the present disclosure and are included to further demonstrate certain aspects of the disclosed compounds and methods, wherein:

FIG. 1 is a schematic flow chart illustrating the step-wise synthesis of N-[4-(2-chloro-benzylsulfanyl)-5-nitro-2-(4-toluenesulfonylamino)-phenyl]-4-toluenesulfonamide (4r) using a process comprising i) 4-toluenesulfonyl chloride, pyridine, 80° C., 82%, ii) fuming HNO₃, acetic acid, 60° C., 36%, and, iii) 2-chlorophenyl-methanethiol, K₂CO₃, reflux, 77%;

FIG. 2 is a schematic flow chart illustrating the step-wise synthesis of N-[5-nitro-4-phenyl-2-(4-toluenesulfonylamino)-phenyl]-4-toluenesulfonamide (8) using a process comprising i) 4-toluenesulfonyl chloride, pyridine, 80° C., 78%, ii) fuming HNO₃, acetic acid, 75° C., 64%, and iii) PhB(OH)₂, Pd(PPh₃)₄, NaHCO₃, DME/H₂O, reflux, 52%;

FIG. 3 is a schematic flow chart illustrating the step-wise synthesis of N-[5-(4-methoxy-phenoxy)-2-(4-toluenesulfonylamino)-phenyl]-4-toluenesulfonamide (12p) using a process comprising i) 4-methoxyphenol, K₂CO₃, DMSO, 130° C., 30%, ii) sodium dithionite, EtOH/H₂O, reflux, iii) 4-toluenesulfonyl chloride, pyridine, 80° C., 20%;

FIG. 4 is a schematic flow chart illustrating the step-wise synthesis of N-[4-(2-chloro-benzyloxy)-2-(4-toluenesulfonylamino)-phenyl]-4-toluenesulfonamide (15d) using a process comprising i) 2-chlorobenzyl chloride, K₂CO₃, acetone, reflux, 86%, ii) sodium dithionite, EtOH/H₂O, reflux, and iii) 4-toluenesulfonyl chloride, pyridine, 80° C., 42%;

FIG. 5 is a schematic flow chart illustrating the step-wise synthesis of N-[4-(imidazol-1-yl)-2-(4-toluenesulfonylamino)-phenyl]-4-toluenesulfonamide using a process comprising i) imidazole, K₂CO₃, DMSO, 130° C., 38%, ii) 3 atm H₂, Pd/C, EtOH, room temperature, and iii) 4-toluenesulfonyl chloride, pyridine, 80° C., 42%;

FIG. 6 is a schematic flow chart illustrating the step-wise synthesis of N-[4-(4-chloro-phenyl)-2-(4-toluenesulfonylamino)-phenyl]-4-toluenesulfonamide (21) using a process comprising i) Br₂, sodium acetate trihydrate, acetic acid, 0° C. to room temperature, 66%, ii) 4-chlorophenylboronic acid, Pd(PPh₃)₄, NaHCO₃, DME/H₂O, reflux, 65%, iii) 3 atm H₂, Pd/C, EtOH, and iv) 4-toluenesulfonyl chloride, pyridine, 80° C., 24%;

FIG. 7 is a schematic flow chart illustrating the step-wise synthesis of N-[2-(4-chloro-phenylsulfanyl)-4,5-bis-(4-toluenesulfonylamino)-phenyl]-acetamide (24) using a process comprising i) Na₂S₂O₄, EtOH/H₂O, reflux, 100%. ii) Ac₂O, DMAP, pyridine, room temperature;

FIG. 8 is a schematic flow chart illustrating the step-wise synthesis of 2-(4-methoxy-phenoxy)-4,5-bis-(4-toluenesulfonylamino)-benzoic acid (34) using a process comprising i) Ac₂O, DMAP, pyridine, room temperature, 100%, ii) HNO₃, Ac₂O, AcOH, 0° C., 22%, iii) NBS, cat. benzoyl peroxide, CCl₄, reflux, 32%, iv) hexamethylenetetramine, CHCl₃, reflux, 23%, v) 2-methyl-2-butene, NaH₂PO₄.H2O, NaClO₂, t-butanol/H₂O, room temperature, 81%, vi) 50% HCl, MeOH, reflux, 90%, vii) 4-methoxyphenol, K₂CO₃, acetone, reflux, 93%, viii) sodium dithionite, EtOH/H₂O, reflux, ix) 4-toluenesulfonyl chloride, pyridine, 80° C., 42%, and x) KOH, MeOH, reflux, 64%;

FIG. 9 is a schematic flow chart illustrating the step-wise synthesis of N-[4-(4-methoxy-phenoxy)-5-methyl-2-(4-toluenesulfonylamino)-phenyl]-4-toluenesulfonamide (37) using a process comprising i) 4-methoxyphenol, K₂CO₃, acetone, reflux, 48%, ii) HCl, MeOH, reflux, 97%, iii) H₂, Pd/C, acetic acid, room temperature, and iv) 4-toluenesulfonyl chloride, pyridine, 80° C.;

FIG. 10 is a schematic flow chart illustrating the step-wise synthesis of [5-(4-methoxy-phenoxy)-2-(4-toluenesulfonylamino)-phenyl]-phosphate (43) using a process comprising i) 4-methoxyphenol, K₂CO₃, DMSO, 140° C., 58%, ii) diethyl chlorophosphate, TEA, toluene, 80° C., 78%, iii) 3 atm H₂, Pd/C, EtOH, room temperature, iv) 4-toluenesulfonyl chloride, pyridine, room temperature, 42% and v) TMSI, CH₃CN, 0° C. to room temperature, 85%;

FIG. 11 is a schematic flow chart illustrating the step-wise synthesis of [4-(4-methoxy-phenoxymethyl)-2-(4-toluenesulfonyloxy)-phenyl]-4-toluenesulfonate (48) using a process comprising i) 4-toluenesulfonyl chloride, pyridine, room temperature, 87%, ii) NaBH₄, EtOH, 0° C. to room temperature, 83%, iii) thionyl chloride, CH₂Cl₂, 45° C., 78%, and iv) 4-methoxyphenol, NaI, K₂CO₃, acetone, reflux, 67%;

FIG. 12 is a schematic flow chart illustrating the step-wise synthesis of N-[4-(4-chloro-phenylsulfanyl)-5-nitro-2-(4-toluoylamino)-phenyl]-4-toluamide (51) using a process comprising i) H₂O/conc., H₂SO₄, 80° C., 88%, ii) 4-toluoyl chloride, pyridine, room temperature, 35%, and iii) 4-chlorothiophenol, NaI, K₂CO₃, acetone, reflux, 62%;

FIG. 13 is a schematic flow chart illustrating the step-wise synthesis of N-[4-(4-chloro-phenylsulfanyl)-5-nitro-2-(4-toluenesulfonyl-methyl-amino)-phenyl]-N-methyl-4-toluenesulfonamide (53) using a process comprising MeI, K₂CO₃, acetone, reflux, 14%;

FIG. 14 is a schematic flow chart illustrating the step-wise synthesis N-[2-amino-4-(4-chloro-phenylsulfanyl)-5-nitro-phenyl]-4-toluenesulfonamide (55) using a process comprising i) 4-toluenesulfonyl chloride, pyridine, 80° C., 60%, and ii) 4-chlorothiophenol, K₂CO₃, acetone, reflux, 43%;

FIG. 15 is a schematic flow chart illustrating the step-wise synthesis of N-[2-hydroxy-4-(4-methoxy-phenoxy)-phenyl]-4-toluenesulfonamide (59) using a process comprising i) N,N-diisopropylethylamine, bromomethyl methyl ether, NaI, DME, room temperature, 98%, ii) sodium dithionite, EtOH/H₂O, reflux, iii) 4-toluenesulfonyl chloride, pyridine, room temperature, 11%, and iv) conc. HCl, ZnCl₂, EtOH, room temperature, 99%; and

FIG. 16 is a schematic flow chart illustrating the synthesis of various Formula V compounds using COCl₂ or SO₂Cl₂ to form a Formula V compound where Y₅ is C═O or SO₂ respectively and starting with a compound where X₅ is —O— and R₁₈ is p-(C₆H₄)OCH₃ as indicated in Table 6 below.

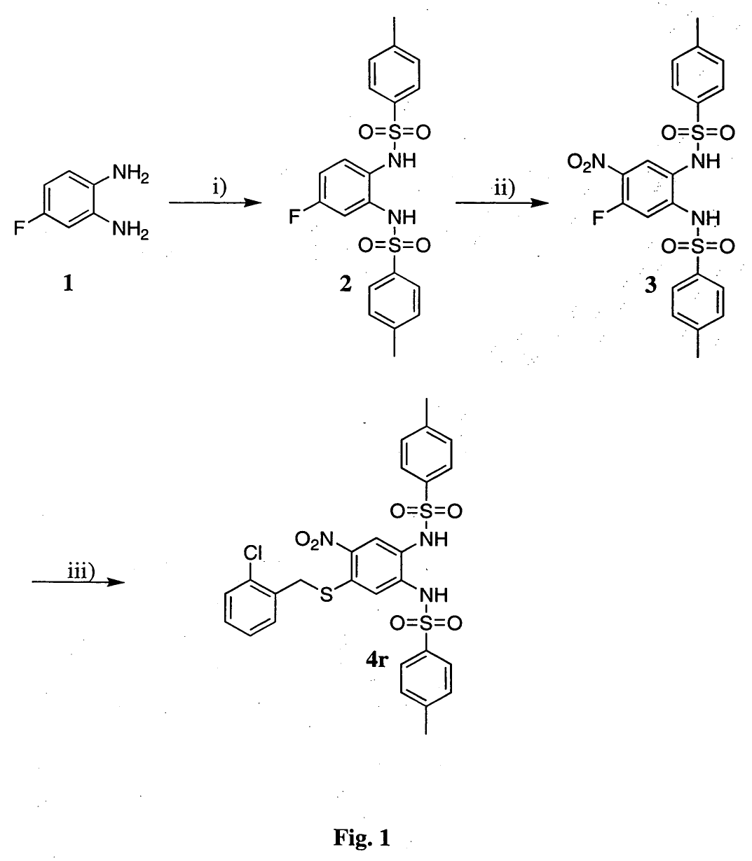

The compounds synthesized using the schemes illustrated in FIGS. 1-15 and other disclosed compounds are listed by compound number in Tables 1-2 below. The compound numbers used in the tables correspond to the numbers used in the drawings.

### DETAILED DESCRIPTION OF THE PRESENTLY PREFERRED EMBODIMENTS

The Src family of PTKs catalyzes the transfer of the gamma phosphate of ATP to protein substrates within the cell. The sulfonamide-based inhibitors act by blocking this transfer of the phosphate thereby inhibiting the catalytic activity of the Src family. These compounds are reversible inhibitors. By blocking the catalytic activity of the Src family, the signal-transduction pathway regulating the growth of tumor cells can be stopped or significantly impeded. The disclosed sulfonamide-based inhibitors show specificity for Src over the two other kinases tested, Csk and FGFr.

Definitions

Hematologic Disease As used herein, “hematologic disease” refers to a disease in which there is abnormal generation of blood cells.

Neurologic Disease As used herein, “neurologic disease” refers to a disease caused by abnormalities within the nervous system.

Proliferative Disease As used herein, “proliferative disease” refers to a disease that occurs due to abnormal growth or extension by the multiplication of cells (Cambridge Dictionary of Biology, 1990).

Autoimmune Disease As used herein, “autoimmune disease” refers to a disease caused by the presence and activation of T or B lymphocytes capable of recognizing “self” constituents with the release of auto-antibodies or damage caused to cells by cell-mediated immunity (Cambridge Dictionary of Biology, 1990).

Allergic/Immunological Disease As used herein, “allergic/immunological disease” refers to disease caused by one or more aspects of the immune system. Examples of included types of diseases are immunodeficiency, characterized by increased susceptibility to infections due to the deficiency of a component of the immune system (B cells, T cells, phagocytic cells, and complement); hypersensitivity disorders which result from immunologically specific interactions between antigens (exogenous or endogenous) and humoral antibodies or sensitized lymphocytes; and reactions to transplantations, in which allografts are rejected through either a cell-mediated or a humoral immune reaction of the recipient against antigens present on the membranes of the donor's cells (The Merck Manual, 1999).

Viral Infection As used herein, “viral infection” refers to a disease caused by the invasion of body tissue by a micro-organism that requires a cell in which to multiply (Cambridge Dictionary of Biology, 1990).

Src family of protein tyrosine kinases As used herein, “Src family of protein tyrosine kinases” refers to a group of intracellular non-receptor tyrosine kinases that share similar structural features and regulation such as a N terminal sequence for lipid attachment, a unique domain, SH3, SH2, and kinase domains, followed by a C-terminal negative regulatory tail (Smithgall, 1998). Any reference to the Src family or its individual members includes all alternatively spliced forms of these proteins. Examples include alternatively spliced neuronal Src and alternatively spliced forms of Fyn and Lyn. Alternatively spliced forms of Src are referred to as Nₓ, where x indicates the size of the N-loop within the SH3 domain where alternative splicing occurs. Therefore, Src is also referred to as N₆. Examples of alternatively spliced forms of Src include N₁₂ and N₂₃.

Src family of tyrosine kinase-related disease As used herein, “Src family of tyrosine kinase-related disease” refers to any disease in which the disorder occurs due to an alteration in the activity of the Src family of tyrosine kinases, or in which it is advantageous to block the signaling pathway of a Src family member.

Binding As used herein, “binding” refers to the non-covalent or covalent interaction of two chemical compounds.

Inhibiting As used herein, “inhibiting” refers to the ability of a substance to reduce the velocity of an enzyme-catalyzed reaction (Biochemical Calculations, 1976). A substance is a better inhibitor than another if it is able to cause the same amount of reduction in velocity at a lower concentration than another substance.

Halogen As used herein, “halogen” refers to fluoro, chloro, bromo, or iodo.

Alkyl As used herein, “alkyl” refers to a group of carbon and hydrogen atoms derived from an alkane molecule by removing one hydrogen atom. “Alkyl” may include saturated monovalent hydrocarbon radicals having straight, cyclic or branched moieties. Said “alkyl” group may include an optional carbon-carbon double or triple bond where said alkyl group comprises at least two carbon atoms. It is understood that for cyclic moieties at least three carbon atoms are required in said alkyl group.

Aryl As used herein, “aryl” refers to an organic radical derived from an aromatic hydrocarbon by removal of one hydrogen.

Alkoxy As used herein, “alkoxy” refers to O-alkyl groups wherein “alkyl” is as defined above.

Hydrogen bond As used herein, “hydrogen bond” refers to the primarily electrostatic bond formed by interaction of a hydrogen atom covalently bound to a highly electronegative element (e.g., oxygen, nitrogen, or fluorine) and a second electronegative atom (e.g., oxygen, nitrogen, or fluorine). The bonding partners are called “hydrogen bond donor atom,” that is the atom to which hydrogen is covalently bound, and “hydrogen bond acceptor atom.”

Salt bridge As used herein, “salt bridge” refers to the attractive force, described by Coulomb's law, between either a cation and an anion or between a cationic and an anionic group of atoms; the cationic and anionic groups may be on the same molecule or on different molecules.

Heterocyclic As used herein, heterocyclic, refers to a cyclic compound in which one or more of the atoms in the ring are elements other than carbon. The atoms that are not carbon may be any possible substituent. Heterocyclic compounds may or may not be aromatic.

Orientation of Compounds

Certain disclosed compounds may exist in different enantiomeric forms. This disclosure relates to the use of all optical isomers and stereoisomers of the disclosed compounds that possess the desired activity. One of skill in the art would be aware that if a given isomer does not possess the desired activity, that isomer should not be used for treatment.

Pharmaceutical Compositions

Pharmaceutically Acceptable Carriers

The disclosed compositions comprise an effective amount of one or more disclosed sulfonamide-based compounds or pharmaceutically acceptable salts thereof, dissolved and/or dispersed in a pharmaceutically acceptable carrier.

The phrases “pharmaceutically and/or pharmacologically acceptable” refer to molecular entities and/or compositions that do not produce an adverse, allergic and/or other unacceptable reaction when administered to an animal.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards. Various pharmaceutical preparations and administration methods are discussed in U.S. Pat. No. 6,503,914 and the references cited therein.

Lipid Formulations and/or Nanocapsules

In certain embodiments, the use of lipid formulations and/or nanocapsules is contemplated for the introduction of with the disclosed sulfonamide-based compounds or pharmaceutically acceptable salts thereof into host cells as disclosed in U.S. Pat. No. 6,503,914.

Kits

Disclosed therapeutic kits comprise the disclosed sulfonamide-based compounds or pharmaceutically acceptable salts thereof. Such kits will generally contain, in suitable container means, a pharmaceutically acceptable formulation of with the disclosed sulfonamide-based compounds in a pharmaceutically acceptable formulation as disclosed in U.S. Pat. No. 6,503,914. The kit may have a single container means, and/or it may have distinct container means for each compound.

Combination Treatments

In order to increase the effectiveness of with the disclosed sulfonamide-based compounds, it may be desirable to combine these compositions with other agents effective in the treatment of the disease as disclosed in U.S. Pat. No. 6,503,914. The disclosed sulfonamide-based compounds may also be combined with other agents, treatments and/or therapies in the treatment of hematologic diseases, osteoporosis, neurological diseases, autoimmune diseases, allergic/immunological diseases, viral infections, and hyperproliferative disease. Such treatments and therapies that may be combined with the use of the disclosed compounds include chemotherapy, radiotherapy, immunotherapy, gene therapy, antisense, inducers of cellular proliferation, inhibitors or cellular proliferation, regulators of programmed cell death, surgery and other agents and treatment as discussed in U.S. Pat. No. 6,503,914, the references cited therein and the references cited herein.

### EXAMPLES

The following examples are included to demonstrate preferred embodiments. It should be appreciated by those skilled in the art that the techniques disclosed in the examples which follow represent techniques discovered by the inventor to function well in the practice of the disclosed techniques, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of this disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of this disclosure.

The number in parentheses next to a compound name, either final product or intermediate, refers to the compound reference numbers used in FIGS. 1-15 and/or Tables 1-6.

### Examples 1 and 2

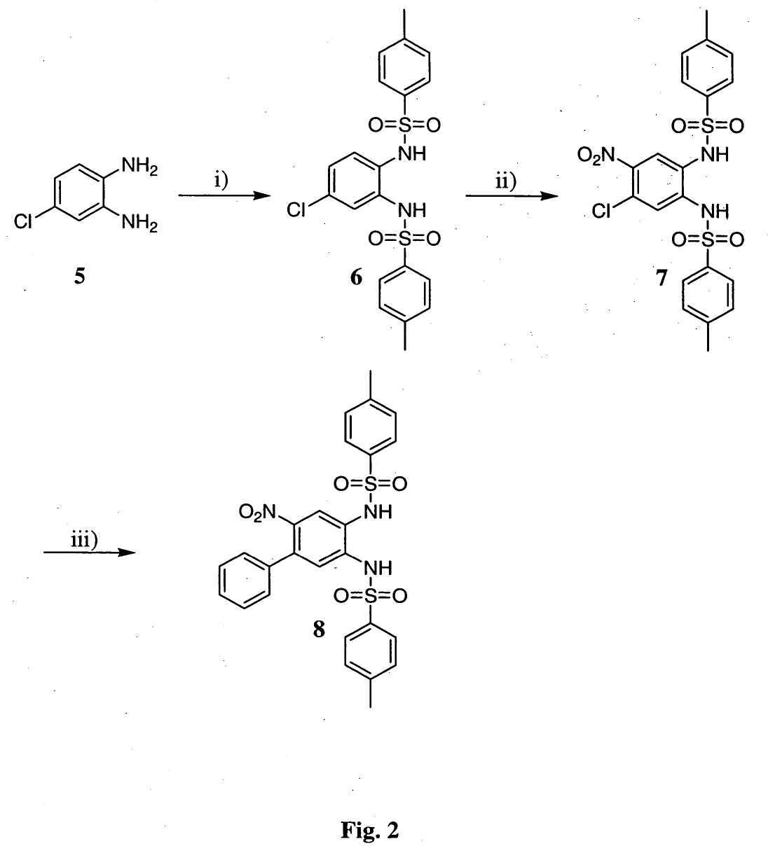

### Synthesis Formula I Compounds (FIGS. 1 and 2)

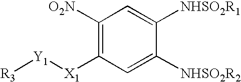

### Example 1

### Representative Synthesis of 4a-4u (Scheme 1; FIG. 1)

4-fluoro-1,2-phenylenediamine (1): Commercially available from Lancaster Synthesis, Windham, N.H., USA:

N-[4-fluoro-2-(4-toluenesulfonylamino)-phenyl]-4-toluenesulfonamide (2): 1.00 g 1 (7.9 mmol) was dissolved in 2 mL anhydrous pyridine. To the solution was added 3.18 g 4-toluenesulfonyl chloride (16.67 mmol, 2.1 eq) dissolved in 7 mL anhydrous pyridine. The solution was heated at 75° C. for 18 hours and then poured into 70 mL ice cold 20% HCl. The resulting solid was collected by vacuum filtration and washed with deionized H₂O. After air drying, the product was recrystallized from 1:9H₂O/acetic acid to afford a brown solid. Yield=2.82 g (82%).

N-[4-fluoro-5-nitro-2-(4-toluenesulfonylamino)-phenyl]-4-toluenesulfonamide (3): 2.77 g 2 (6.4 mmol) was suspended in 20 mL glacial acetic acid and heated to 60° C. To the suspension was added a solution of 0.7 mL filming HNO₃ (15.95 mmol, 2.5 eq) in 1 mL glacial acetic acid. One half of the fuming HNO₃ was added in one portion, and the remainder was slowly added dropwise. After 5 minutes, a thick orange precipitate had formed. Stirring was continued one hour more at 60° C., and the solid was collected by vacuum filtration. The resulting yellow solid was washed with deionized H₂O and air dried. Purification was completed by recrystallization from EtOH. Yield=1.11 g (36%).

N-[4-(2-chloro-benzylsulfanyl)-5-nitro-2-(4-toluenesulfonylamino)-phenyl]-4-toluenesulfonamide (4r): 30 mg 3 (0.063 mmol) was dissolved in 5 mL acetone, and to the solution was added 16.5 μL 2-chlorophenyl-methanethiol (0.125 mmol, 2.0 eq) and 52 mg K₂CO₃ (0.378 mmol, 6 eq). The reaction mixture was refluxed for 4 days at which time conversion of starting material was complete. The crude reaction mixture was diluted with EtOAc/10% HCl, and the EtOAc extract was washed once more with 10% HCl and twice with deionized H₂O. After drying over anhydrous Na₂SO₄ and filtering the EtOAc extract was stripped to dryness and then recrystallized from CHCl₃/hexanes to give a yellow solid. Yield=30 mg (77%).

### Example 2

### Representatitve Synthesis of Compound 8 (Scheme 2; FIG. 2)

4-chloro-1,2-phenylenediamine (5): Commercially available from Sigma-Aldrich Chemical Company, Milwaukee, Wis., USA.

N-[4-chloro-2-(4-toluenesulfonylamino)-phenyl]-4-toluenesulfonamide (6): 6.00 g 5 (42.1 mmol) was dissolved in 12 mL anhydrous pyridine. To the solution was added 16.45 g 4-toluenesulfonyl chloride (16.67 mmol, 2.1 eq) dissolved in 30 mL anhydrous pyridine. The solution was heated at 75° C. for 18 hours and then poured into 200 mL ice cold 20% HCl. The resulting purplish-black solid was collected by vacuum filtration and washed with deionized H₂O. After air drying, the product was recrystallized from 1:9H₂O/acetic acid to afford a purplish-red solid. Yield=14.83 g (78%).

N-[4-chloro-5-nitro-2-(4-toluenesulfonylamino)-phenyl]-4-toluenesulfonamide (7): 3.00 g 6 (6.4 mmol) was suspended in 12 mL glacial acetic acid and heated to 70° C. To the suspension was added a solution of 0.7 mL fuming HNO₃ (15.95 mmol, 2.5 eq) in 1 mL glacial acetic acid. One half of the fuming HNO₃ was added in one portion, and the remainder was slowly added dropwise. After 5 minutes, a thick orange precipitate had formed. Stirring was continued 45 minutes more at 70° C., and the solid was collected by vacuum filtration. The resulting yellow solid was washed with deionized H₂O and air dried. Purification was completed by recrystallization from 9:1 acetic acid:H₂O. Yield 2.11 g (64%).

N-[5-nitro-4-phenyl-2-(4-toluenesulfonylamino)-phenyl]-4-toluenesulfonamide (8): 25 mg 7 (0.051 mmol) and 6 mg Pd(PPh₃)₄ were dissolved in 1 ml DME. To the solution was added 0.2 mL 1 M NaHCO₃ (0.20 mmol, 4 eq) and 9 mg PhB(OH)₂ (0.071 mmol, 1.4 eq). The reaction was refluxed for 21 hours, at which time reaction progress appeared to have stalled. An additional 5 mg PhB(OH)₂ (0.75 eq) and 6 mg Pd(PPh₃)₄ (0.1 eq) were added, and reflux was maintained for an additional 6 hours. After cooling to room temperature, the reaction was diluted with EtOAc, and the EtOAc extract was washed with 10% HCl, followed by subsequent washes with deionized H₂O and saturated aqueous NaCl. The extract was dried over anhydrous Na₂SO₄, filtered, and concentrated by rotary evaporation. The resulting reddish-orange solid was recrystallized from CHCl₃/hexanes. A light-brown solid was isolated via filtration. Yield=14 mg (51%).

Compounds 9a-9k were synthesized by Tripos Receptor Research, Bude, Cornwall, UK.

### Examples 3-6

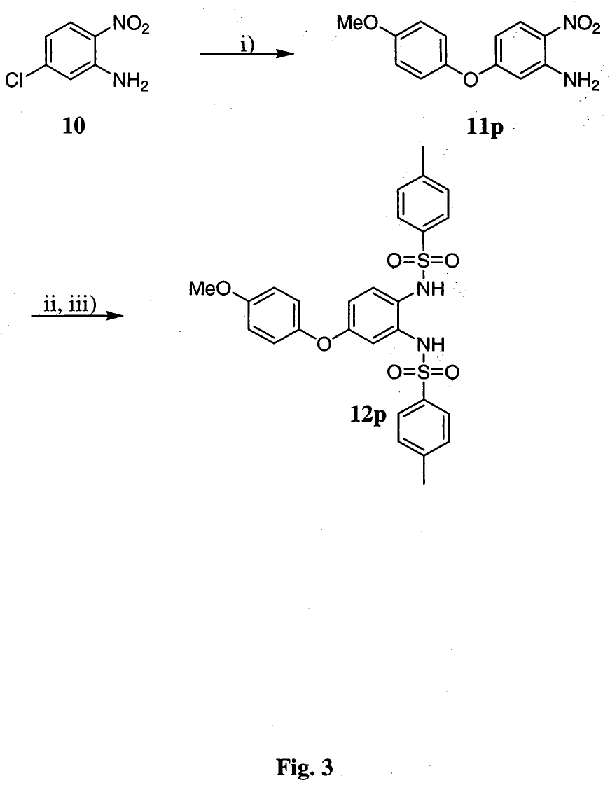

### Synthesis of Formula II Compounds (FIGS. 3-6)

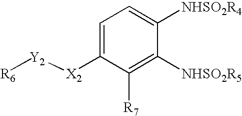

### Example 3

### Representative Synthesis of 12a-12hh (Scheme 3; FIG. 3)

5-chloro-2-nitroaniline (10; see FIG. 3): Commercially available from Sigma-Aldrich Chemical Company, Milwaukee, Wis., USA.

5-(4-methoxy-phenylsulfanyl)-2-nitroaniline (11gg): 100 mg of 5-chloro-2-nitroaniline (10) (0.58 mmol) and 144 μL 4-methoxybenzenethiol (1.17 mmol, 2 eq) were dissolved in 2 mL DMSO. To the solution was added 480 mg K₂CO₃ (3.48 mmol, 6 eq), and the suspension was heated at 130° C. for 15 hours, at which time consumption of the 5-chloro-2-nitroaniline (10) was indicated. The reaction mixture was diluted with EtOAc/deionized H₂O, and the EtOAc extract was washed three times more with deionized H₂O and twice with saturated aqueous NaCl. After drying over anhydrous Na₂SO₄ and filtering, the EtOAc extract was concentrated by rotary evaporation to give a red-brown solid. Purification was completed by recrystallization from CHCl₃/hexanes. An orange powder was obtained after filtration and further washing of the precipitate with hexanes. Yield=113 mg (71%).

N-[5-(4-methoxy-phenylsulfanyl)-2-(4-toluenesulfonylamino)-phenyl]-4-toluenesulfonamide (12gg): 25 mg of 5-(4-methoxy-phenylsulfanyl)-2-nitroaniline (0.091 mmol) was suspended in 3 mL EtOH/1 mL deionized H₂O, and the suspension was heated to near boiling. To the hot suspension was added 158 mg sodium dithionite (0.91 mmol, 10 eq). The reaction mixture was refluxed for 18 hours, and then an additional 53 mg sodium dithionite (3.3 eq) was added and reflux continued. After an additional 30 minutes of reflux, the reaction mixture was cooled to room temperature and poured into excess saturated aqueous NaHCO₃ solution. The product was extracted into EtOAc, and the extract was washed once more with saturated aqueous NaHCO₃ and once with deionized H₂O. After drying over anhydrous Na₂SO₄ and filtering, the product was collected as a brown-black solid following rotary evaporation.

The crude product was mixed with 38 mg 4-toluenesulfonyl chloride (0.20 mmol, 2.2 eq) and dissolved in 1 mL anhydrous pyridine. The reaction mixture was heated at 80° C. for 18 hours and then poured into 10 mL 20% HCl. A brown solid was isolated after filtering and washing with deionized H₂O. The solid was redissolved in 1:9 deionized H₂O/acetic acid, and deionized H₂O was added until precipitation occurred. A tan solid was isolated after filtering and washing with deionized H₂O. Yield=10 mg (20%).

5-(4-methoxy-phenoxy)-2-nitroaniline (11p; see FIG. 3): 100 mg of (0.58 mmol) and 149 mg 4-methoxyphenol (1.16 mmol, 2 eq) were dissolved in 2 mL DMSO. To the solution was added 480 mg K₂CO₃ (3.48 mmol, 6 eq), and the suspension was heated at 130° C. for 2.5 days. The reaction mixture was then diluted with EtOAc/deionized H₂O, and the EtOAc extract was separated and washed three times more with deionized H₂O and twice with saturated aqueous NaCl. After drying over anhydrous Na₂SO₄ and filtering, the EtOAc extract was concentrated by rotary evaporation to give a red-brown solid. Purification was completed recrystallization from CHCl₃/hexanes. An orange powder was obtained after filtration and further washing of the precipitate with hexanes. Yield=46 mg (30%).

N-[5-(4-methoxy-phenoxy)-2-(4-toluenesulfonylamino)-phenyl]-4-toluenesulfonamide (12p): 25 mg 11p (0.091 mmol) was suspended in 3 mL EtOH/1 mL deionized H₂O, and the suspension was heated to near boiling. To the hot suspension was added 158 mg sodium dithionite (0.91 mmol, 10 eq). The reaction mixture was refluxed for 18 hours, and then an additional 53 mg sodium dithionite (3.3 eq) was added and reflux continued. After an additional 30 minutes of reflux, the reaction mixture was cooled to room temperature and poured into excess saturated aqueous NaHCO₃. The product was extracted into EtOAc, and the extract was washed once more with saturated aqueous NaHCO₃ and once with deionized H₂O. After drying over anhydrous Na₂SO₄ and filtering, the product was collected as a brown-black solid following rotary evaporation.

The crude product was mixed with 38 mg 4-toluenesulfonyl chloride (0.20 mmol, 2.2 eq) and dissolved in 1 mL anhydrous pyridine. The reaction mixture was heated at 80° C. for 18 hours and then poured into 10 mL 20% HCl. A brown solid was isolated after filtering and washing with deionized H₂O. The solid was redissolved in 1:9 deionized H₂O/acetic acid, and deionized H₂O was added until precipitation occurred. A tan solid was isolated after filtering and washing with deionized H₂O. Yield=10 mg (20%).

4-amino-3-nitrophenol (13): Commercially available from Sigma-Aldrich Chemical Company, Milwaukee, Wis., USA.

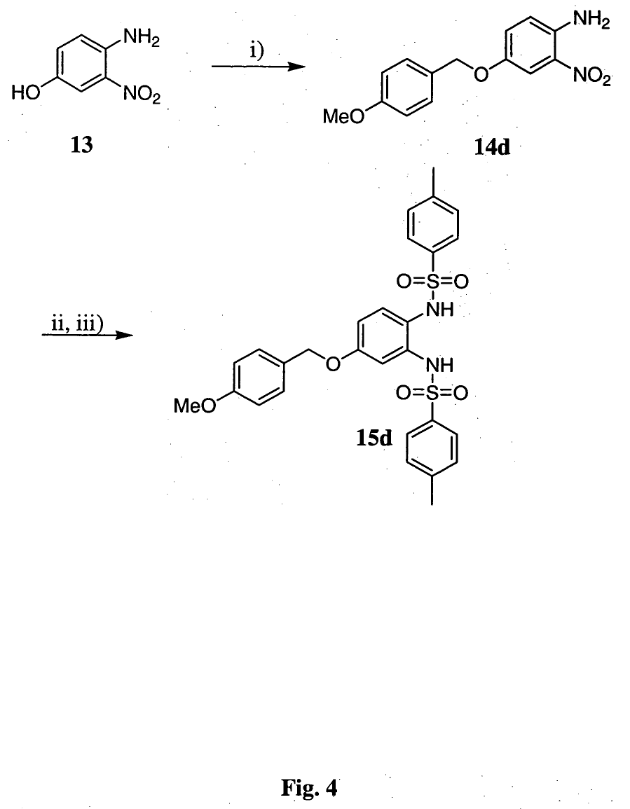

4-(2-chloro-benzyloxy)-2-nitroaniline (14d; see FIG. 4): 75 mg of 4-amino-3-nitrophenol (13) (0.49 mmol) and 94 mg 2-chlorobenzyl chloride (0.58 mmol, 1.2 eq) were dissolved in 2 mL acetone. To the solution was added 350 mg K₂CO₃ (2.45 mmol, 5 eq), and the reaction mixture was refluxed for 18 hours. The reaction was diluted with EtOAc, and the EtOAc solution was washed three times with deionized H₂O. The extract was dried over anhydrous Na₂SO₄, filtered and reduced to dryness by rotary evaporation. The residue was recrystallized from CHCl₃/hexanes to yield an orange solid. Yield=117 mg (86%).

N-[4-(2-chloro-benzyloxy)-2-(4-toluenesulfonylamino)-phenyl]-4-toluenesulfonamide (15d): 25 mg of 4-(2-chloro-benzyloxy)-2-nitroaniline (14d) (0.090 mmol) was dissolved in 3 mL EtOH/1 ml deionized H₂O and heated to near boiling. To the hot solution was added 156 mg sodium dithionite (0.90 mmol, 10 eq), and the solution was heated to reflux. After refluxing 4 hours, an additional 80 mg sodium dithionite (5 eq) was added, and reflux was maintained for an additional four hours. After cooling to room temperature, the reaction mixture was diluted with EtOAc and washed twice with saturated aqueous NaHCO₃. After drying over anhydrous Na₂SO₄, the EtOAc extract was filtered, reduced to dryness and redissolved in 1 mL anhydrous pyridine. To the pyridine solution was added 43 mg 4-toluenesulfonyl chloride (0.23 mmol, 2.5 eq), and the reaction mixture was heated at 80° C. overnight. After overnight heating, the reaction mixture was poured into 20 mL 20% HCl, and a tan solid was collected via filtration. The crude solid was redissolved in a minimum of 1:1 1 M NaOH/EtOH and filtered. The filtered solution was acidified with 20% HCl and a tan solid precipitated. The solid was collected via filtration, washed with deionized H₂O, and air dried. Yield=21 mg (42%).

### Example 5

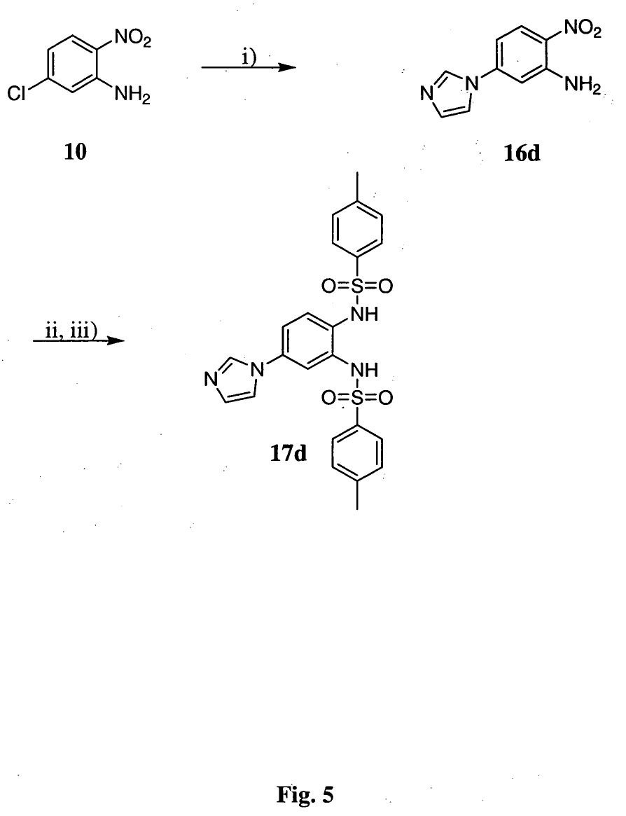

### Representative Synthesis of 17a-f (Scheme 5; FIG. 5)

5-(imidazol-1-yl)-2-nitroaniline (16d; see FIG. 5): 100 mg of 5-chloro-2-nitroaniline (10) (0.58 mmol), 80 mg imidazole (1.18 mmol, 2 eq), and 480 mg K₂CO₃ (3.48 mmol, 6 eq) were mixed in 2 mL DMSO. The reaction mixture was heated at 130° C. for 2.5 days and then diluted with EtOAc/H₂O. The EtOAc phase was washed twice with deionized H₂O and twice with saturated aqueous NaCl solution. After drying over anhydrous Na₂SO₄ and filtering, the extract was concentrated by rotary evaporation to give a yellow-orange solid. The crude solid was recrystallized from CHCl₃/hexanes to provide pure product as an orange solid. Yield=45 mg (38%).

N-[4-(imidazol-1-yl)-2-(4-toluenesulfonylamino)-phenyl]-4-toluenesulfonamide (17d): 15 mg of 5-(imidazol-1-yl)-2-nitroaniline (16d) (0.074 mmol) was dissolved in 2 mL EtOH, and to the solution was added 5 mg Pd/C. The suspension was then shaken for 6 hours under 3 atm H₂ in a Parr shaker and thereafter filtered through Celite. The filtrate was reduced to dryness by rotary evaporation and combined with 31 mg 4-toluenesulfonyl chloride (0.16 mmol, 2.2 eq). The solids were dissolved in 1 mL pyridine, and the reaction mixture was heated at 80° C. for 18 hours. After 18 hours, the pyridine solution was poured into 10 mL 20% HCl. A yellow precipitate formed initially before rapidly redissolving. The pH of the aqueous solution was adjusted to ˜6 with 1 M NaOH, and a yellow solid precipitated. The solid was collected by filtration, washed several times with deionized H₂O, and air dried. Yield=16 mg (42%).

### Example 6

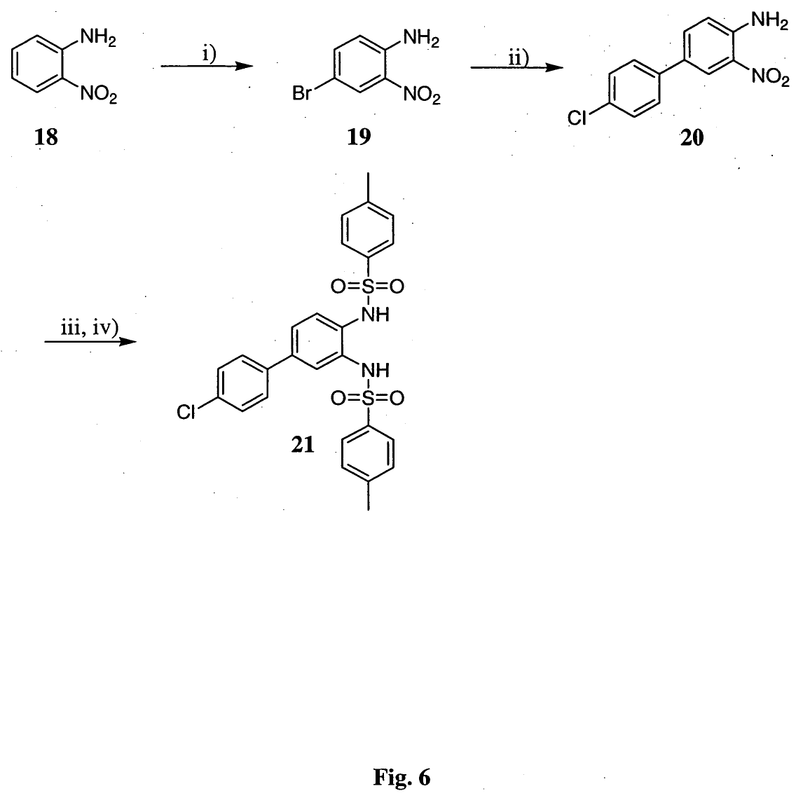

### Synthesis of 21 (Scheme 6; FIG. 6)

2-nitroaniline (18; see FIG. 6): Commercially available from Sigma-Aldrich Chemical Company, Milwaukee, Wis., USA.

4-bromo-2-nitroaniline (19): 2.50 g of 2-nitroaniline (18) (18.1 mmol) and 3.94 g sodium acetate trihydrate (29.0 mmol, 1.6 eq) were dissolved in 25 mL glacial acetic acid. The solution was chilled to 0° C., and a solution of 0.95 ml Br₂ (18.6 mmol, 1.03 eq) in 2 mL acetic acid was added dropwise over 10 minutes. The reaction mixture was warmed to room temperature and then stirred for one hour. After one hour, the reaction mixture was poured into 200 mL ice cold deionized H₂O, and a bright orange solid was collected via filtration. The crude solid was recrystallized from EtOH and air dried to provide an orange crystalline solid. Yield=2.58 g (66%).

4-(4-chlorophenyl)-2-nitroaniline (20): 50 mg of 4-bromo-2-nitroaniline (19) (0.23 mmol) and 27 mg Pd(PPh₃)₄ (0.023 mmol, 0.1 eq) were dissolved in 1 mL DME. To the solution was added 50 mg 4-chlorophenylboronic acid (0.32 mmol, 1.4 eq) and 0.92 mL 1 M NaHCO₃ (0.92 mmol, 4 eq). The reaction mixture was refluxed for four hours, cooled to room temperature, and diluted with EtOAc. The EtOAc solution was washed once with saturated NaHCO₃, once with deionized H₂O, and once with saturated aqueous NaCl. After drying over anhydrous Na₂SO₄ and filtering, an orange solid was isolated by rotary evaporation. The solid was recrystallized from CHCl₃/hexanes to give an orange powder. Yield=37 mg (65%).

N-[4-(4-chloro-phenyl)-2-(4-toluenesulfonylamino)-phenyl]-4-toluenesulfonamide (21): 10 mg 20 (0.040 mmol) was dissolved in 1.5 mL EtOH, and to the solution was added 2 mg Pd/C. The suspension was shaken under 3 atm H₂ in a Parr shaker for 5 hours and then filtered through Celite. The filtrate was reduced to dryness by rotary evaporation, and the crude solid was combined with 18 mg 4-toluenesulfonyl chloride (0.090 mmol, 2.2 eq). The solids were dissolved in 1 mL pyridine, and the solution was heated at 80° C. for 20 hours. After 20 hours, the reaction mixture was poured into 20 mL 10% HCl, and a light brown solid precipitated. The crude solid was redissolved in a minimum of aqueous 10% HCl/EtOH (9:1), and additional 10% HCl was added until precipitation of a tan solid was complete. The solid was isolated via filtration and washed with deionized H₂O. Yield=5 mg (24%).

Compounds 22b-22nn were synthesized by Tripos Receptor Research, Bude, Cornwall, UK.

### Examples 7-9

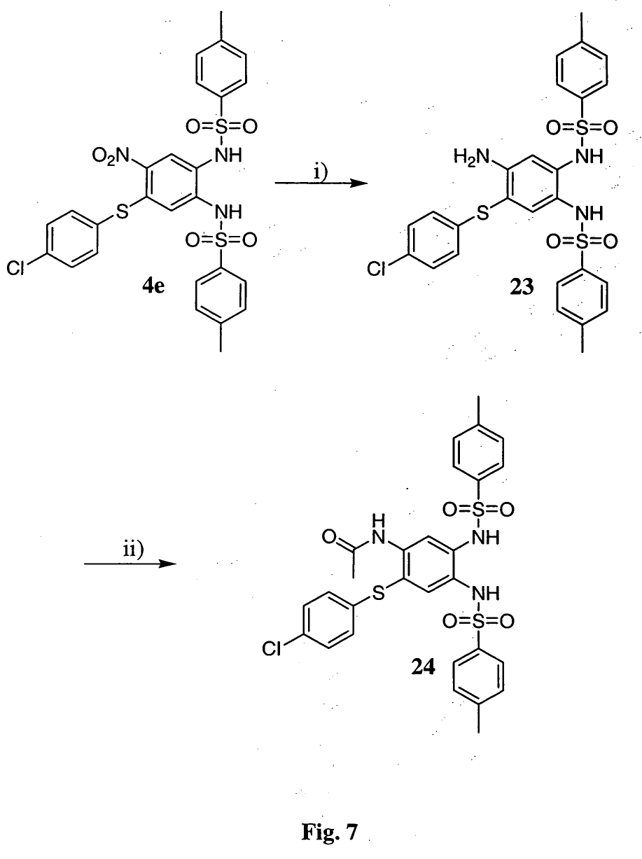

### Synthesis of Formula III Compounds (FIGS. 7-9)

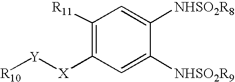

### Example 7

### Synthesis of 23 and 24 (Scheme 7; FIG. 7)

N-[5-amino-4-(4-chloro-phenylsulfanyl)-2-(4-toluenesulfonylamino)-phenyl]-4-toluenesulfonamide (23): 22 mg of N-[4-(4-chloro-phenylsulfanyl)-5-nitro-2-(4-toluenesulfonylamino)-phenyl]-4-toluenesulfonamide (4e) (0.033 mmol) was suspended in 3 mL EtOH/1.5 mL H₂O and heated to near reflux. To the hot suspension was added 59 mg sodium dithionite (0.33 mmol, 10 eq), and within 5 minutes a homogenous solution had formed. After refluxing for 1 hour, an additional 30 mg sodium dithionite (5 eq) was added and reflux was continued for 30 minutes more. The reaction was cooled to room temperature and diluted with CH₂Cl₂/saturated aqueous NaCl. The CH₂Cl₂ phase was washed twice with 10% HCl, once with saturated NaHCO₃, and once with deionized H₂O. The extract was dried over anhydrous Na₂SO₄, filtered and rotary evaporated to a light yellow solid. Yield=24 mg (>100%).

N-[2-(4-chloro-phenylsulfanyl)-4,5-bis-(4-toluenesulfonylamino)-phenyl]-acetamide (24): 19 mg of N-[5-amino-4-(4-chloro-phenylsulfanyl)-2-(4-toluenesulfonylamino)-phenyl]-4-toluenesulfonamide (23) (0.033 mmol) and 0.4 mg 4-dimethylaminopyridine (0.0033 mmol, 0.1 eq) were dissolved in 1 mL pyridine. To the solution was added 31 μL Ac₂O (0.33 mmol, 10 eq), and the reaction mixture was stirred overnight at room temperature. After overnight stirring, the reaction mixture was diluted with EtOAc and washed once with deionized H₂O, three times with 10% aqueous CuSO₄, and twice more with deionized H₂O. The crude extract was dried over anhydrous Na₂SO₄, filtered, and reduced to dryness by rotary evaporation. The crude solid was recrystallized from CHCl₃/hexanes to provide a small amount of reddish-brown solid. Yield not determined.

### Example 8

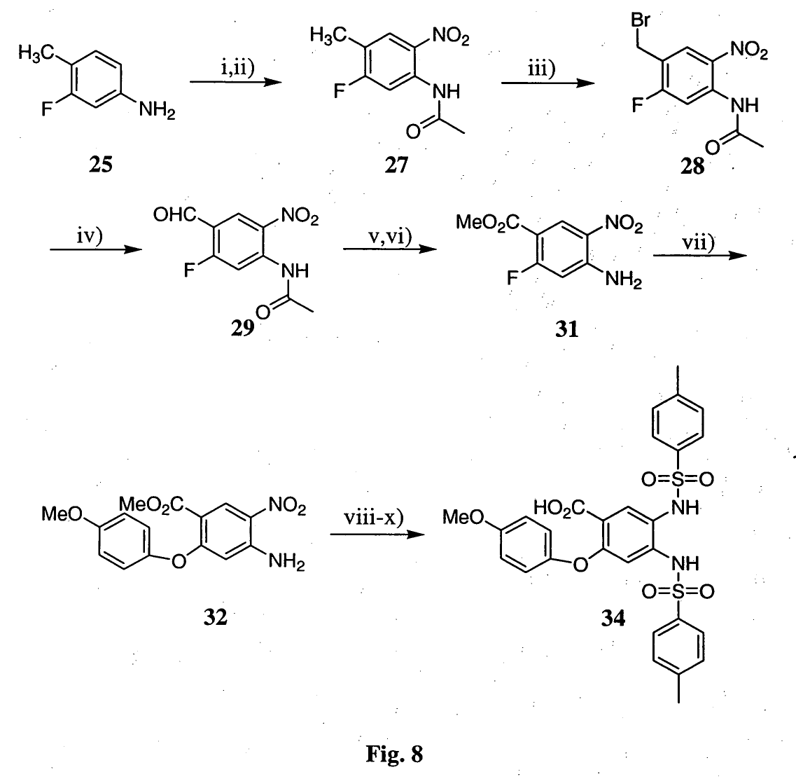

### Synthesis of 33 and 34 (Scheme 8; FIG. 8)

3-fluoro-4-methylaniline (25): Commercially available from Sigma-Aldrich Chemical Company, Milwaukee, Wis., USA.

(3-fluoro-4-methyl-phenyl)-acetamide (26): 5.00 g of 3-fluoro-4-methylaniline (25) (40.0 mmol) and 500 mg 4-dimethylaminopyridine (4.0 mmol, 0.1 eq) were dissolved in 16 mL pyridine. To the solution was added 23 mL Ac₂O (240 mmol, 6 eq). Cooling in an ice bath was necessary immediately following addition to moderate the initial rise in temperature. After the initial cooling period, the reaction was stirred at room temperature for 4 hours, and the pyridine solution was then diluted with EtOAc/H₂O. The organic phase was washed twice more with deionized H₂O, three times with 10% aqueous CuSO₄, and two further times with deionized H₂O. The extract was dried over anhydrous Na₂SO₄, filtered, and reduced to a white solid after rotary evaporation. Yield=6.69 g (100%).

(5-fluoro-4-methyl-2-nitro-phenyl)-acetamide (27): 3.00 g of (3-fluoro-4-methyl-phenyl)-acetamide (26) (18.0 mmol) was dissolved in 4 mL glacial acetic acid/7 mL Ac₂O. The solution was chilled to −5° C., and a solution of 1.0 mL. HNO₃ in 1.5 mL Ac₂O was added dropwise. After addition of the HNO₃ solution was completed, the reaction was maintained at 0° C. for 1 hour and then poured into 60 mL deionized H₂O. A yellow-orange oil separated and solidifed to a yellow solid. The solid was collected by filtration, and washed once with deionized H₂O, once with isopropanol, and twice with hexanes. The product was then chromatographed with 1:7 EtOAc/hexanes to remove residual starting material. The purified product was a yellow solid. Yield=0.84 g (22%).

N-(4-bromomethyl-5-fluoro-2-nitro-phenyl)-acetamide (28): 1.15 g of (5-fluoro-4-methyl-2-nitro-phenyl)-acetamide (27) (5.42 mmol), 1.07 g NBS (5.97 mmol, 1.1 eq), and 33 mg benzoyl peroxide (0.136 mmol, 0.025 eq) were dissolved in 30 mL CCl₄. The reaction mixture was refluxed for 22 hours, cooled to room temperature, filtered through Celite, and reduced to dryness by rotary evaporation. The crude material was chromatographed with 10% EtOAc/hexanes to provide pure product as a yellow solid. Yield=0.50 g (32%).

N-(5-fluoro-4-formyl-2-nitro-phenyl)-acetamide (29): 0.52 g of N-(4-bromomethyl-5-fluoro-2-nitro-phenyl)-acetamide (28) (1.78 mmol) and 0.30 g hexamethylenetetramine (2.14 mmol, 1.2 eq) were dissolved in CHCl₃ and refluxed for 18 hours. After 18 hours, 30 mL glacial acetic acid was added, and reflux was continued for one hour more. The reaction mixture was cooled to room temperature and diluted with EtOAc. The combined organic solution was washed three times with 10% HCl, three times with saturated aqueous NaHCO₃, and twice with deionizized H₂O. After drying over anhydrous Na₂SO₄ and filtering, the organic phase was reduced to dryness to yield a yellow solid. Yield=93 mg (23%).

4-acetylamino-2-fluoro-5-nitro-benzoic acid (30): 93 mg of N-(5-fluoro-4-formyl-2-nitro-phenyl)-acetamide (29) (0.41 mmol) was dissolved in 8 mL t-butanol. To the solution was added 1.1 mL 2-methyl-2-butene (10.3 mmol, 25 eq), followed by a solution of 396 mg NaH₂PO₄.H₂O (2.87 mmol, 7 eq) and 371 mg NaClO₂ (4.1 mmol, 10 eq) in 4 mL H₂O. The reaction mixture was stirred at room temperature for 2.5 days and then concentrated by rotary evaporation. After dilution with deionized H₂O, the aqueous phase was washed twice with hexanes and then acidified with 10% HCl. The product was extracted into EtOAc, and the organic extract was washed twice with deionized H₂O. After drying over anhydrous Na₂SO₄ and filtering, the product was isolated by rotary evaporation to yield a tan solid. Yield=80 mg (81%).

Methyl 4-amino-2-fluoro-5-nitro-benzoate (31): 80 mg of 4-acetylamino-2-fluoro-5-nitro-benzoic acid (30) (0.33 mmol) was dissolved in 20 mL MeOH, and 1 mL 50% aqueous HCl was added. The reaction mixture was refluxed for 18 hours and neutralized with saturated aqueous NaHCO₃. MeOH was removed by rotary evaporation, and the remaining aqueous solution was extracted with EtOAc. The organic extract was washed twice more with saturated aqueous NaHCO₃ and twice with deionized H₂O. After drying over anhydrous Na₂SO₄ and filtering, the product was isolated as a yellow solid. Yield=64 mg (90%).

Methyl 4-amino-2-(4-methoxy-phenoxy)-5-nitro-benzoate (32): 64 mg of methyl 4-amino-2-fluoro-5-nitro-benzoate (31) (0.30 mmol) and 74 mg 4-methoxyphenol (0.60 mmol, 2 eq) were dissolved in 10 mL acetone. To the solution was added 250 mg K₂CO₃ (1.8 mmol, 6 eq), and the suspension was refluxed for 2 days. The reaction was cooled to room temperature and diluted with EtOAc/deionized H₂O. The EtOAc phase was washed twice with 1 M NaOH and twice with deionized H₂O. After drying over anhydrous Na₂SO₄ and filtering, the product was isolated as a yellow solid after rotary evaporation. Yield=88 mg (93%).

Methyl 2-(4-methoxy-phenoxy)-4,5-bis-(4-toluenesulfonylamino)-benzoate (33): 44 mg of methyl 4-amino-2-(4-methoxy-phenoxy)-5-nitro-benzoate (32) (0.14 mmol) was dissolved in 5 mL EtOH/2 mL deionized H₂O and heated to reflux. To the refluxing solution was added 240 mg sodium dithionite (1.4 mmol, 10 eq), and reflux was continued for 3 hours more. Upon consumption of the starting material, the reaction mixture was cooled to room temperature and diluted with EtOAc. The organic phase was washed twice with saturated aqueous NaHCO₃ and once with saturated aqueous NaCl solution. The extract was dried over Na₂SO₄, filtered, and rotary evaporated to a brown oil. The crude product was redissolved in 1 mL pyridine, and 68 mg 4-toluenesulfonyl chloride (0.35 mmol, 2.5 eq) was added. After heating at 80° C. for 2.5 days, the reaction mixture was poured into 20 mL 10% HCl. A tan solid was collected and washed with deionized H₂O. The tan solid was redissolved in a minimum of EtOH/1 M NaOH. The solution was filtered into 20 mL 10% HCl, and a tan solid again formed. The precipitate was collected via filtration and washed with deionized H₂O. Yield=35 mg (42%).

2-(4-methoxy-phenoxy)-4,5-bis-(4-toluenesulfonylamino)-benzoic acid (34): 29 mg of methyl 2-(4-methoxy-phenoxy)-4,5-bis-(4-toluenesulfonylamino)-benzoate (33) (0.049 mmol) and 27 mg KOH (0.49 mmol, 10 eq) were dissolved in 2.5 mL MeOH. The reaction mixture was refluxed for two days, at which time consumption of starting material was indicated by TLC. The reaction mixture was cooled to room temperature and diluted with EtOAc/deionized H₂O. The aqueous phase was extracted once more with EtOAc and then was made acidic with 10% HCl. The organic extracts were discarded. The acidified aqueous solution was extracted with EtOAc, and the yellow extract was washed once with deionized H₂O. After drying over anhydrous Na₂SO₄ and filtering, a tan solid was obtained after solvent evaporation. The crude material was redissolved in 10 mL 1 M NaOH, and the solution was poured into 20 mL 10% HCl. A tan precipitate was isolated after filtering and washing with deionized H₂O. Yield=18 mg (64%).

### Example 9

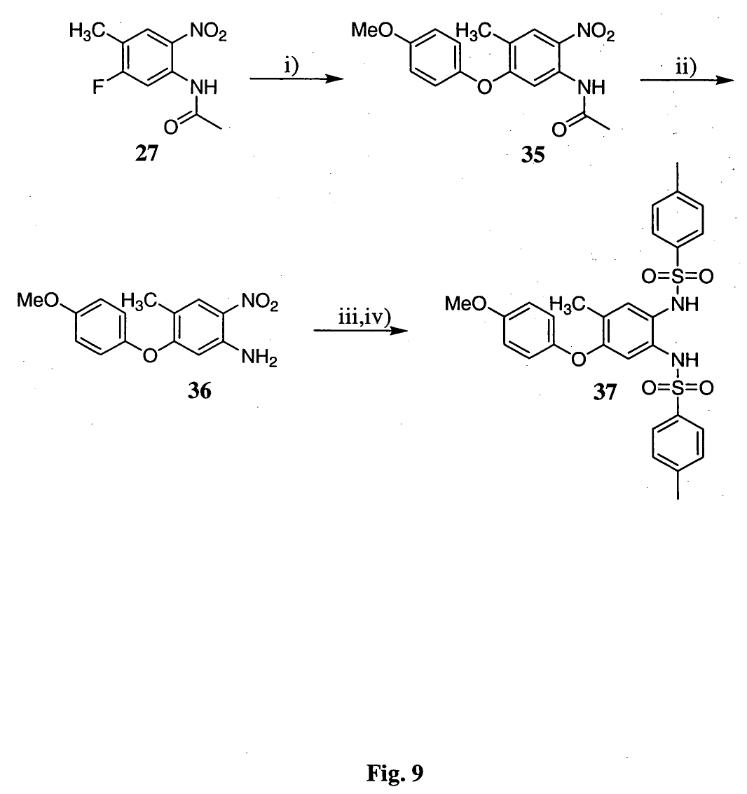

### Synthesis of 37 (Scheme 9; FIG. 9)

[5-(4-methoxy-phenoxy)-4-methyl-2-nitro-phenyl)-acetamide (35): 1.50 g of (5-fluoro-4-methyl-2-nitro-phenyl)-acetamide (27) (7.1 mmol) and 1.73 g 4-methoxyphenol (14.2 mmol, 2 eq) were dissolved in 30 mL acetone. To the solution was added 7.8 g K₂CO₃ (56.5 mmol, 8 eq), and the suspension was refluxed for 5 hours. After cooling to room temperature, the reaction mixture was diluted with EtOAc/deionized H₂O. The EtOAc phase was successively washed twice with deionized H₂O, twice with 1 M NaOH, once with 10% HCl, once with deionized H₂O, and twice with saturated aqueous NaCl. After drying over anhydrous Na₂SO₄ and filtering, a brownish-orange solid was obtained after rotary evaporation. The solid was recrystallized from CHCl₃/hexanes, and the recrystallized product was purified further by column chromatography (15% EtOAc/hexanes, increasing to 33% EtOAc/hexanes). A final recrystallization from EtOAc/hexanes yielded a brilliant orange solid pure product after filtration. Yield=1.07 g (48%).

5-(4-methoxy-phenoxy)-4-methyl-2-nitro-aniline (36): 500 mg of [5-(4-methoxy-phenoxy)-4-methyl-2-nitro-phenyl)-acetamide (35) (1.58 mmol) was dissolved in 10 mL MeOH, and to the solution was added 20 mL 25% HCl (60.5 mmol, 38 eq). The combined solution was refluxed for 4 hours and then cooled to room temperature. The reaction mixture was diluted with EtOAc/deionized H₂O, and the EtOAc phase was washed twice with saturated aqueous NaHCO₃ and once with deionized H₂O before drying over anhydrous Na₂SO₄. After filtration and removal of solvent by rotary evaporation, an oily orange solid was obtained. Yield=419 mg (97%).

N-[4-(4-methoxy-phenoxy)-5-methyl-2-(4-toluenesulfonylamino)-phenyl]-4-toluenesulfonamide (37): 200 mg of 5-(4-methoxy-phenoxy)-4-methyl-2-nitro-aniline (36) (0.73 mmol) was dissolved in 5 mL glacial acetic acid. To the solution was added 20 mg Pd/C, and the suspension was shaken for 2 days under 3 atm H₂. The reaction mixture was filtered through Celite, and the acetic acid was removed by rotary evaporation: Remaining traces of acetic acid were removed by rotary evaporation of the residual solid from toluene (acetic acid/toluene azeotrope) and drying under vacuum.

The crude brown oil from above was redissolved in 4 mL pyridine, and 350 mg 4-toluenesulfonyl chloride (1.84 mmol, 2.5 eq.) was added to the solution. The reaction mixture was heated at 80° C. for 2.5 days and then poured into 30 mL 20% HCl. A tan solid was isolated after filtering and washing with deionized H₂O. The crude solid was chromatographed with 3:7 EtOAc/hexanes, followed by 1:1 to separate the product from a closely-eluting impurity. The product was isolated as a tan foam after rotary evaporation. Yield not determined.

N-[4,5-dibromo-2-(4-toluenesulfonylamino)-phenyl]-4-toluenesulfonamide (38a): Commercially available from Sigma-Aldrich Chemical Company, Milwaukee, Wis., USA.

Compounds 38b-38d were synthesized by Tripos Receptor Research, Bude, Cornwall, UK.

### Examples 10-15

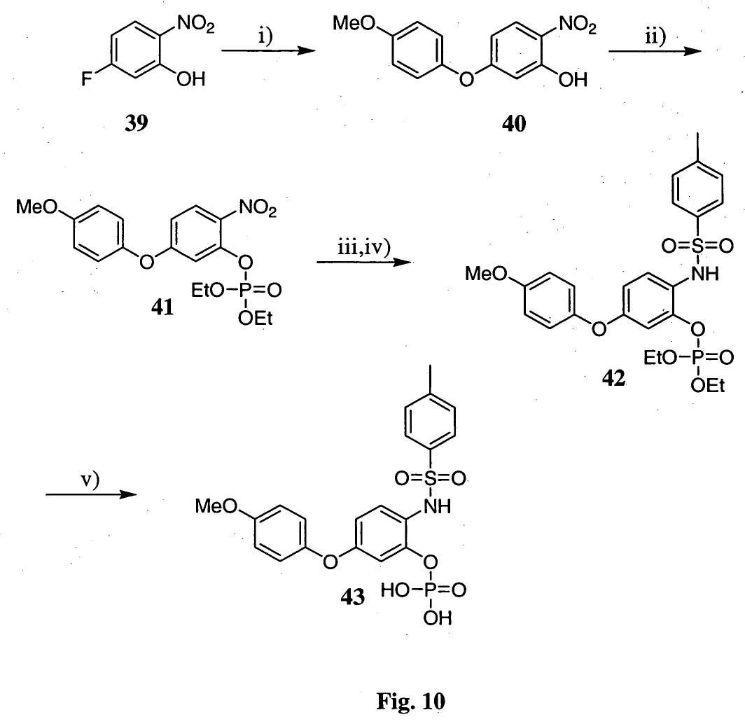

### Synthesis of Formula IV Compounds (FIG. 10-15)

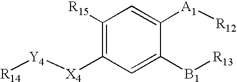

### Example 10

### Synthesis of 42 and 43 (Scheme 10; FIG. 10)

5-fluoro-2-nitrophenol (39): Commercially available from Sigma-Aldrich Chemical Company, Milwaukee, Wis., USA.

5-(4-methoxy-phenoxy)-2-nitrophenol (40): 400 mg of 5-fluoro-2-nitrophenol (39) (2.55 mmol) and 632 mg 4-methoxyphenol (5.1 mmol, 2 eq) were dissolved in 10 mL DMSO. To the solution was added 2.80 g K₂CO₃ (20.4 mmol, 8 eq), and the suspension was heated at 140° C. for 3.5 hours. The reaction mixture was then cooled to room temperature and diluted with EtOAc/10% HCl. The EtOAc phase was washed three additional times with deionized H₂O and twice with saturated aqueous NaCl. After drying over anhydrous Na₂SO₄ and filtering, a brown oil was obtained after rotary evaporation. The product was isolated by chromatography with 10% EtOAc/hexanes. A yellow solid was obtained after removal of solvent by rotary evaporation. Yield=384 mg (58%).

Diethyl [5-(4-methoxy-phenoxy)-2-nitro-phenyl]-phosphate (41): 70 mg of 5-(4-methoxy-phenoxy)-2-nitrophenol (40) (0.268 mmol) was dissolved in 2 mL anhydrous toluene. To the solution was added 35 μL diethyl chlorophosphate (0.282 mmol, 1.05 eq) and 39 μL TEA (0.282 mmol, 1.05 eq). The turbid yellow solution was heated at 80° C. for 42 hours. After 42 hours, the reaction mixture was filtered through Celite, and the filtrate was diluted with EtOAc and extracted twice with saturated aqueous Na₂CO₃. The organic phase was subsequently washed twice with 10% HCl and twice with deionized H₂O. Washing of the organic phase was then continued with saturated aqueous Na₂CO₃ followed by deionized H₂O until residual 40 was removed. After drying over anhydrous Na₂SO₄ and filtering, the crude product was obtained as a yellow oil after rotary evaporation. Yield=83 mg (78%).

Diethyl [5-(4-methoxy-phenoxy)-2-(4-toluenesulfonylamino)-phenyl]-phosphate (42): 249 mg of diethyl[5-(4-methoxy-phenoxy)-2-nitro-phenyl]-phosphate (41) (0.63 mmol) was dissolved in 5 mL EtOH. To the solution was added 50 mg Pd/C. The suspension was shaken under 3 atm H₂ for 18 hours and then filtered through Celite. The filtrate was diluted with EtOAc and washed successively with saturated aqueous Na₂CO₃, deionized H₂O, saturated aqueous Na₂CO₃, and finally three times with deionized H₂O. After drying over anhydrous Na₂SO₄ and filtering, the crude product was obtained as a brown oil after rotary evaporation.

The brown oil was redissolved in 3 mL pyridine, and 182 mg 4-toluenesulfonyl chloride (0.94 mmol, 1.5 eq) was added to the solution. The reaction mixture was stirred at room temperature for 4 days and then diluted with EtOAc/deionized H₂O. The EtOAc phase was washed once more with deionized H₂O, four times with 10% aqueous CuSO₄, and twice more with deionized H₂O. After drying over anhydrous Na₂SO₄ and filtering, the crude product was obtained as a brown oil after rotary evaporation. The crude product was chromatographed with 1:1 EtOAc/hexanes to provide pure product as a yellow oil after rotary evaporation. Yield=168 mg (51%).

[5-(4-methoxy-phenoxy)-2-(4-toluenesulfonylamino)-phenyl]-phosphate (43): 45 mg of diethyl[5-(4-methoxy-phenoxy)-2-(4-toluenesulfonylamino)-phenyl]-phosphate (42) (0.086 mmol) was dissolved in 1 mL anhydrous CH₃CN. The solution was chilled to 0° C., and 51 μL (0.36 mmol, 4.2 eq) TMSI was added to the reaction mixture, and the solution was warmed gradually to room temperature and stirred for 20 hours. After 20 hours, the reaction was quenched with MeOH and diluted with EtOAc/deionized H₂O. The EtOAc phase was extracted twice with 0.1 M NaOH, and the aqueous phase was then immediately acidified and extracted with EtOAc. The EtOAc extract was washed once more with deionized H₂O, dried over anhydrous Na₂SO₄, filtered, and reduced to dryness by rotary evaporation to provide a pale yellow solid. Yield=34 mg (85%).

### Example 11

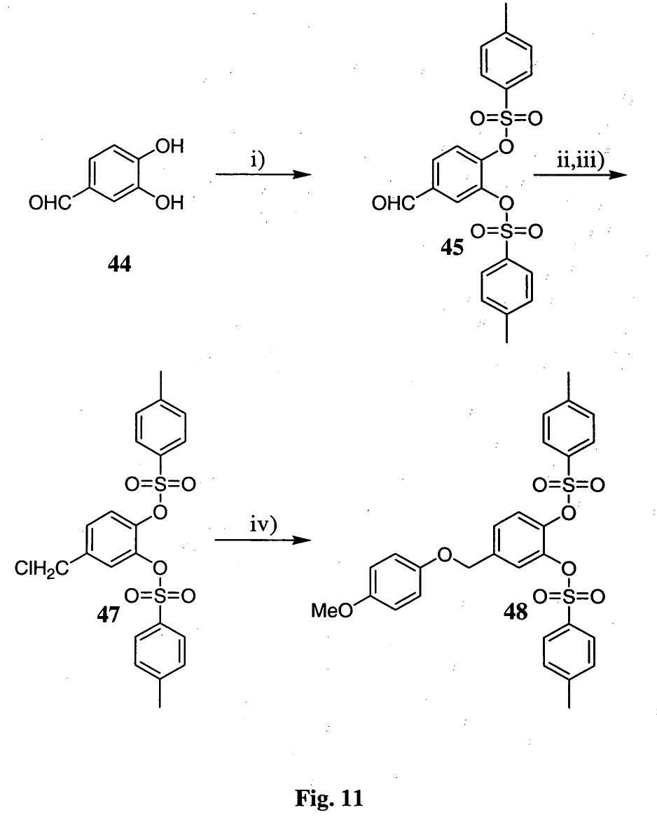

### Synthesis of 48 (Scheme 11; FIG. 11)

3,4-dihydroxybenzaldehyde (44): Commercially available from Sigma-Aldrich Chemical Company, Milwaukee, Wis., USA.

[4-formyl-2-(4-toluenesulfonyloxy)-phenyl]-4-toluenesulfonate (45): 600 mg of 3,4-dihydroxybenzaldehyde (44) (4.35 mmol) and 1.99 g 4-toluenesulfonyl chloride (10.43 mmol, 2.4 eq) were dissolved in 10 mL anhydrous pyridine. The reaction mixture was stirred at room temperature for 2.5 days and then diluted with EtOAc/deionized H₂O. The EtOAc phase was washed successively with deionized H₂O, saturated aqueous Na₂CO₃, four times with 10% aqueous CuSO₄, once more with deionized H₂O, once more with saturated aqueous Na₂CO₃, and once with deionized H₂O. After drying over anhydrous Na₂SO₄ and filtering, the crude product was isolated as a brown oil. The product was purified by chromatography with 1:3 EtOAc/hexanes to yield an oily yellow solid after removal of solvent by rotary evaporation. Yield=1.68 g (87%).

[4-hydroxymethyl-2-(4-toluenesulfonyloxy)-phenyl]-4-toluenesulfonate (46): 200 mg of [4-formyl-2-(4-toluenesulfonyloxy)-phenyl]-4-toluenesulfonate (45) (0.45 mmol) was dissolved in 8 mL EtOH. The solution was chilled to 0° C., and 21 mg NaBH₄ (0.54 mmol, 1.2 eq) was added to the reaction mixture. The reaction was stirred at 0° C. for 1 hour and then warmed to room temperature and stirred for 17 hours. The reaction was then diluted with EtOAc/deionized H₂O, and the organic phase was washed twice more with deionized H₂O and dried over anhydrous Na₂SO₄. After filtration, the crude product was isolated as a colorless oil after rotary evaporation. The crude product was chromatographed with 40% EtOAc/hexanes to afford pure product as a colorless oil. Yield=167 mg (83%).

[4-chloromethyl-2-(4-toluenesulfonyloxy)-phenyl]-4-toluenesulfonate (47): 44 mg of [4-hydroxymethyl-2-(4-toluenesulfonyloxy)-phenyl]-4-toluenesulfonate (46) (0.098 mmol) was dissolved in 3 mL CH₂Cl₂, and 57 μL thionyl chloride (0.78 mmol, 8 eq) was added to the solution. The reaction mixture was heated at 45° C. for 24 hours and then cooled to room temperature. After cooling to room temperature, the reaction mixture was diluted with EtOAc and washed twice with saturated aqueous Na₂CO₃ and once with deionized H₂O. After drying over anhydrous Na₂SO₄ and filtering, the solvent was removed by rotary evaporation. The crude product was chromatographed with 20% EtOAc/hexanes to afford pure product as a pale yellow oil. Yield=36 mg (78%).

[4-(4-methoxy-phenoxymethyl)-2-(4-toluenesulfonyloxy)-phenyl]-4-toluenesulfonate (48): 56 mg of [4-chloromethyl-2-(4-toluenesulfonyloxy)-phenyl]-4-toluenesulfonate (47) (0.12 mmol) and 18 mg 4-methoxyphenol (0.14 mmol, 1.2 eq) were dissolved in 2 mL acetone. To the solution was added 5 mg NaI (0.03 mmol, 0.25 eq) and 99 mg K₂CO₃ (0.72 mmol, 6 eq), and the reaction mixture was refluxed for 25 hours. After 25 hours, the reaction was cooled to room temperature and diluted with EtOAc/deionized H₂O. The organic phase was washed once with 1 M NaOH and once with deionized H₂O. After drying over anhydrous Na₂SO₄ and filtering, a yellow-green oil was obtained following rotary evaporation. The crude product was chromatographed with 1:3 EtOAc/hexanes to afford pure product as a white solid. Yield=45 mg (67%).

### Example 12

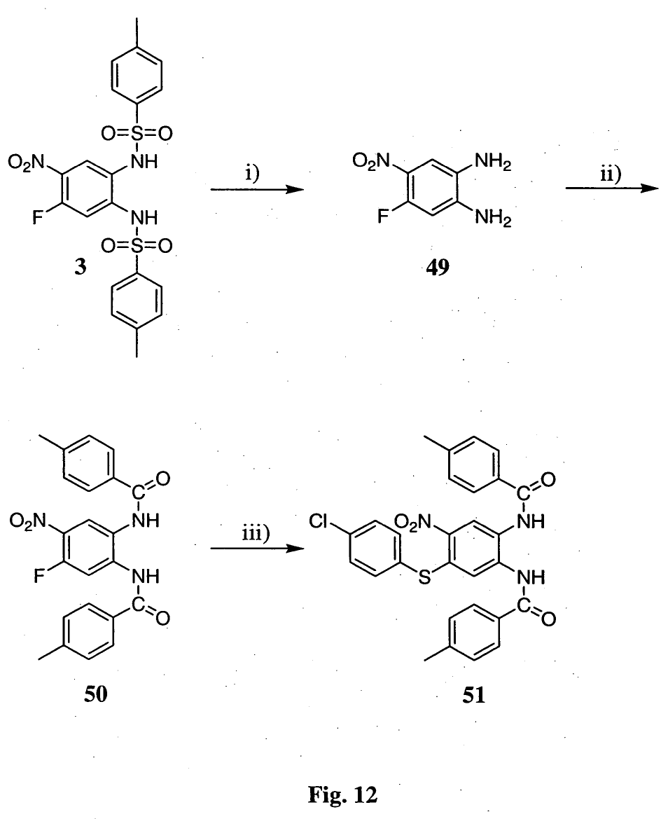

### Representative Synthesis of 51 and 52 (Scheme 12; FIG. 12)

4-fluoro-5-nitro-1,2-phenylenediamine (49): 158 mg of N-[4-fluoro-5-nitro-2-(4-toluenesulfonylamino)-phenyl]-4-toluenesulfonamide (3) (0.33 mmol) was suspended in a solution of 35 μL deionized H₂O/360 μL concentrated H₂SO₄, and the suspension was heated at 80° C. for one hour. After one hour, the reaction mixture was poured into 10 mL deionized H₂O, and a yellow solid precipitated. The aqueous suspension was heated to boiling to redissolve the solids and then cooled to room temperature. After adjusting the solution pH to ˜9 with concentrated NH₄OH, an orange solid precipitated. The solid was isolated by filtration, washed with deionized H₂O, and air dried. Yield=49 mg (88%).

N-[4-fluoro-5-nitro-2-(4-toluoylamino)-phenyl]-4-toluamide (50): 40 mg of 4-fluoro-5-nitro-1,2-phenylenediamine (49) (0.23 mmol) was dissolved in 1 mL anhydrous pyridine, and to the solution was added 70 μL 4-toluoyl chloride (0.53 mmol, 2.25 eq). The solution was stirred for 19 hours at room temperature and then poured into 20 mL 0.25 M NaOH. An orange solid was isolated by filtration and subsequently recrystallized from EtOH. Pure product was isolated by filtration after chilling in ice. Yield=33 mg (35%).

N-[4-(4-chloro-phenylsulfanyl)-5-nitro-2-(4-toluoylamino)-phenyl]-4-toluamide (51): 10 mg of N-[4-fluoro-5-nitro-2-(4-toluoylamino)-phenyl]-4-toluamide (50) (0.025 mmol) and 7 mg 4-chlorothiophenol (0.049 mmol, 2 eq) were dissolved in 5 mL acetone. To the suspension was added 23 mg K₂CO₃ (0.17 mmol, 6.7 eq), and the suspension was refluxed for 26 hours. The reaction mixture was then diluted with EtOAc/deionized H₂O, and the organic phase was washed once with 10% HCl and twice with deionized H₂O. After drying over anhydrous Na₂SO₄ and filtering, the solvent was removed by rotary evaporation. The crude product was recrystallized from CHCl₃/hexanes to afford a yellow-green solid after filtration. Yield=8 mg (62%).

### Example 13

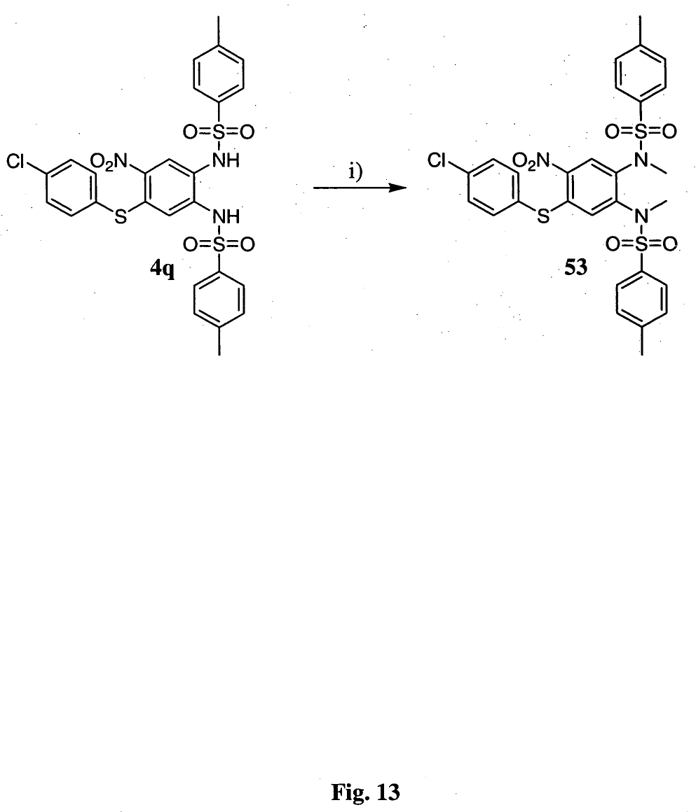

### Synthesis of 53 (Scheme 13; FIG. 13)

N-[4-(4-chloro-phenylsulfanyl)-5-nitro-2-(4-toluenesulfonyl-methyl-amino)-phenyl]-N-methyl-4-toluenesulfonamide (53): 20 mg of N-[4-(4-chloro-benzylsulfanyl)-5-nitro-2-(4-toluenesulfonylamino)-phenyl]-4-toluenesulfonamide (4q) (0.033 mmol) and 40 μL MeI (0.663 mmol, 20 eq) were dissolved in 4 mL acetone. To the solution was added 46 mg K₂CO₃ (0.33 mmol, 10 eq), and the resulting suspension was refluxed for 48 hours. After cooling to room temperature, the reaction was diluted with EtOAc/10% HCl. The organic phase was washed twice with 10% HCl and twice with deionized H₂O. There was a substantial amount of yellow solid that was insoluble in either phase that was removed by filtration subsequent to drying. The EtOAc phase was dried over anhydrous Na₂SO₄, filtered, and rotary evaporated to a yellow solid. After recrystallization from CHCl₃/hexanes, a small quantity of yellow solid was collected and washed with hexanes. Yield=3 mg (14%).

### Example 14

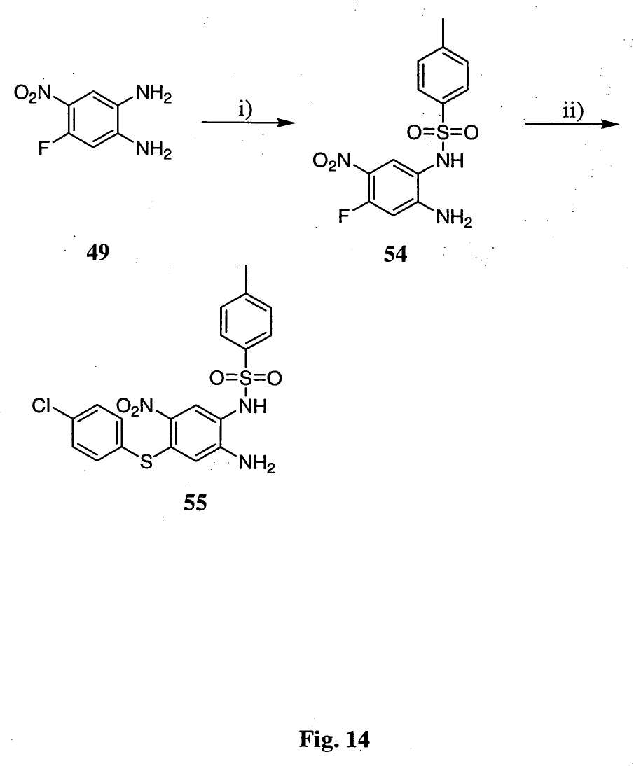

### Representative Synthesis of 55 and 56 (Scheme 14; FIG. 14)

N-(2-amino-4-fluoro-5-nitro-phenyl)-4-toluenesulfonamide (54): 21 mg of 4-fluoro-5-nitro-1,2-phenylenediamine (49) (0.23 mmol) and 31.5 mg 4-toluenesulfonyl chloride (0.16 mmol, 1.3 eq) were dissolved in 1 mL pyridine, and the solution was heated at 80° C. After 18 hours heating, the hot reaction mixture was poured into 10 mL 20% HCl to produce a brown precipitate. After recrystallization from 1:9H₂O/AcOH, a tan solid was isolated after filtration. Yield=25 mg (60%).

N-[2-amino-4-(4-chloro-phenylsulfanyl)-5-nitro-phenyl]-4-toluenesulfonamide (55): 15 mg of N-(2-amino-4-fluoro-5-nitro-phenyl)-4-toluenesulfonamide (54) (0.046 mmol) and 13 mg 4-chlorothiophenol (0.092, 2 eq) were dissolved in 5 mL acetone. To the solution was added 37 mg K₂CO₃ (0.27 mmol, 5.8 eq), and the suspension was refluxed for 23 hours. After cooling to room temperature, the reaction mixture was diluted with EtOAc/deionized H₂O. The organic phase was washed twice more with deionized H₂O, dried over anhydrous Na₂SO₄, and filtered. After recrystallization from CHCl₃/hexanes and filtration, the product was obtained as a tan solid. Yield=9 mg (43%).

### Example 15

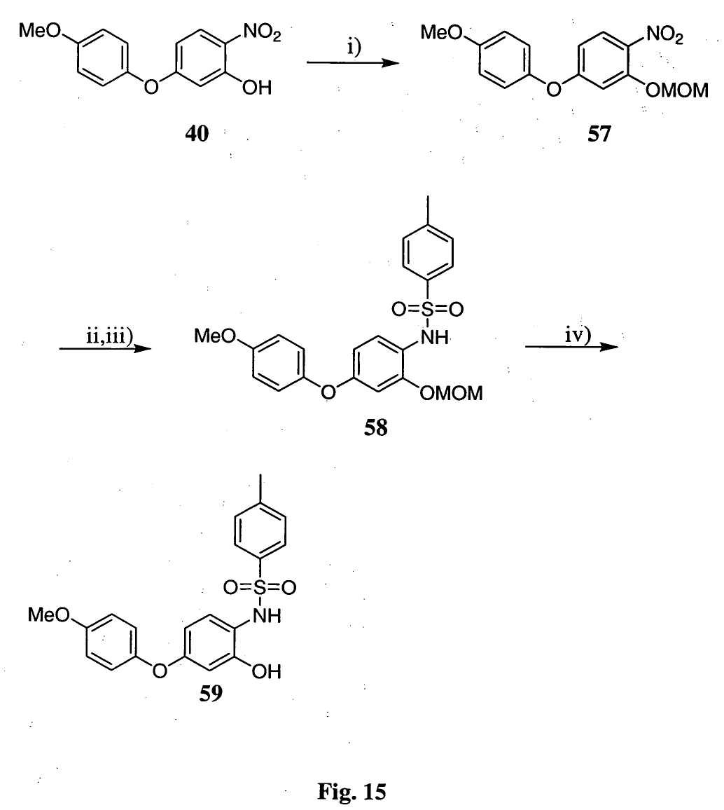

### Representative Synthesis of 59-65 (Scheme 15; FIG. 15)

2-methoxymethoxy-4-(4-methoxy-phenoxy)-1-nitro-benzene (57): 120 mg of 5-(4-methoxy-phenoxy)-2-nitrophenol (40) (0.46 mmol) and 190 mg NaI (1.27 mmol, 2.75 eq) were dissolved in 5 mL anhydrous DME. To the solution was added 140 μL N,N-diisopropylethylamine (0.80 mmol, 1.75 eq) and 50 μL bromomethyl methyl ether (0.58 mmol, 1.26 eq). The reaction mixture was stirred at room temperature for 2 hours and then diluted with EtOAc/deionized H₂O. The organic phase was washed twice successively with saturated aqueous Na₂CO₃ followed by deionized H₂O. After drying over anhydrous Na₂SO₄ and filtering, the product was obtained as a yellow solid following rotary evaporation. Yield=137 mg (98%).

N-[2-methoxymethoxy-4-(4-methoxy-phenoxy)-phenyl]-4-toluenesulfonamide (58): 137 mg of 2-methoxymethoxy-4-(4-methoxy-phenoxy)-1-nitro-benzene (57) (0.45 mmol) was suspended in 4 mL EtOH/2 mL deionized H₂O. The suspension was heated to near boiling, and 782 mg sodium dithionite (4.5 mmol, 10 eq) was added in small portions. The reaction mixture was heated at reflux for 2 hours, and 300 mg fresh sodium dithionite (4 eq) was added. An additional 200 mg fresh sodium dithionite (2.5 eq) was added one hour later, and reflux was continued for 18 hours. After 18 hours, the reaction was cooled to room temperature and diluted with EtOAc. The EtOAc solution was washed twice with saturated aqueous NaHCO₃, dried over anhydrous Na₂SO₄, filtered, and rotary evaporated to dryness.

The crude reduction product and 108 mg 4-toluenesulfonyl chloride were dissolved in 2 mL pyridine, and the reaction mixture was stirred at room temperature for 2.5 days. The reaction mixture was diluted with EtOAc and washed with deionized water, and the organic phase was then washed 4 times with 10% aqueous CuSO₄, and twice more with deionized H₂O. After drying over anhydrous Na₂SO₄ and filtering, the crude product was obtained as a brown oil following rotary evaporation. Purification of the product was completed by column chromatography with 1:3 EtOAc/hexanes. Yield=22 mg (11%).

N-[2-hydroxy-4-(4-methoxy-phenoxy)-phenyl]-4-toluenesulfonamide (59): 22 mg of N-[2-methoxymethoxy-4-(4-methoxy-phenoxy)-phenyl]-4-toluenesulfonamide (58) (0.051 mmol) was dissolved in 5 mL EtOH. To the yellow solution was added 0.5 mL concentrated HCl (excess), and 15 mg ZnCl₂ (0.11 mmol, 2.2 eq). The reaction was stirred at room temperature for 24 hours, and then diluted with EtOAc/deionized H₂O. The organic phase was washed once more with deionized H₂O and twice with saturated aqueous NaHCO₃. After drying over anhydrous Na₂SO₄ and filtering, the solvent was removed by rotary evaporation. Purification of the product was completed via column chromatography with 20% EtOAc/hexanes, followed by 33% EtOAc/hexanes, to provide the product as a white solid. Yield=17 mg (99%).

Compounds 71a-71nn were synthesized by Tripos Receptor Research, Bude, Cornwall, UK.

### Example 16

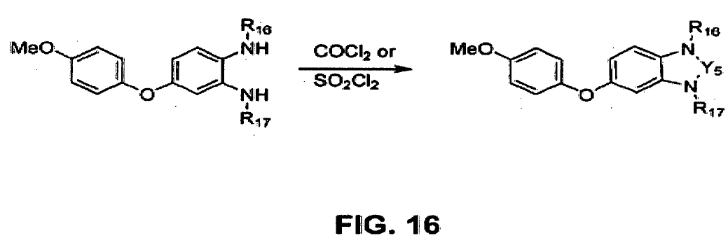

### (Hypothetical): Synthesis of Formula V Compounds (FIG. 16)

As shown in FIG. 16, Formula V compounds can be synthesized using COCl₂ or SO₂Cl₂ to form a Formula V compound where Y₅ is C═O or SO₂ respectively. While the scheme illustrated in FIG. 16 starts with a compound where X₅ is —O— and R₁₈ is p-(C₆H₄)OCH₃ as indicated in Table 6 below, the starting compound may be synthesized by one skilled in the art from compound 11p (Example 3, FIG. 3) or from compound 40 (Example 15, FIG. 15).

A listing of compounds that can be synthesized using schemes 1-15 as illustrated in Examples 1-15 and FIGS. 1-15 is provided in Table 1 below.

TABLE 1

| | | | | Purity |
|---|---|---|---|---|
| # | 1H NMR | Name | MS | by HPLC |
| 2 | (300MHz, acetone-d6): 7.91(d, | N-[4-fluoro-5-nitro-2-(4- | ND | ND |
| | J=8.2Hz, 2H), 7.59(d, J=8.4Hz, | toluenesulfonylamino)- | | |
| | 2H), 7.39-7.48(m, | phenyl]-4- | | |
| | 6H), 2.43(m, 6H) | toluenesulfonamide | | |
| 4a | (300MHz, acetone-d6): | N-[4-(4-methoxy- | M−1=598(ESI−) | >94% |
| | 7.59-7.66(m, 3H), 7.47(d, J=6Hz, | phenylsulfanyl)-5-nitro-2-(4- | | |
| | 2H), 7.36-7.42(m, | toluenesulfonylamino)- | | |
| | 6H), 7.23(d, J=6Hz, 2H), | phenyl]-4- | | |
| | 6.90(s, 1H, 3.93(s, 3H), 2.45(s, | toluenesulfonamide | | |
| | 3H), 2.43(s, 3H) | | | |
| 4b | (300MHz, acetone-d6): 7.76(dt, | N-[4-(2-methoxy- | M−1=598(ESI−) | >79% |
| | J=7.5Hz, J′=2Hz, 1 | phenylsulfanyl)-5-nitro-2-(4- | | |
| | H), 7.56-7.63(m, 3H), 7.53(dd, | toluenesulfonylamino)- | | |
| | J=7.5Hz, J′=2Hz, | phenyl]-4- | | |
| | 1H), 7.37-7.47(m, 3H), 7.21-7.39(m, | toluenesulfonamide | | |
| | 5H), 6.90(s, 1H), | | | |
| | 3.82(s, 3H), 2.40(m, 6H) | | | |
| 4c | (300MHz, acetone-d6): | N-[4-(3-methoxy- | M−1=598(ESI−) | >90% |
| | 7.56-7.65(m, 4H), 7.33-7.47(m, | phenylsulfanyl)-5-nitro-2-(4- | | |
| | 6H), 7.28(dd, J=7.5Hz, | toluenesulfonylamino)- | | |
| | J′=2Hz, 1H), 7.13(m, 2H), | phenyl]-4- | | |
| | 6.97(s, 1H), 3.92(s, 3H), | toluenesulfonamide | | |
| | 2.42(s, 6H) | | | |
| 4d | (300MHz, acetone-d6): | N-[4-(4-hydroxy- | M−1=584(ESI−) | <70% |
| | 7.57-7.68(m, 3H), 7.34-7.46(m, | phenylsulfanyl)-5-nitro-2-(4- | | |
| | 8H), 7.12(d, J=9Hz, | toluenesulfonylamino)- | | |
| | 2H), 6.92(s, 1H), 2.41(m, | phenyl]-4- | | |
| | 6H) | toluenesulfonamide | | |
| 4e | (300MHz, acetone-d6): | N-[4-(4-chloro- | M−1=602(ESI−) | >97% |
| | 7.67-7.73(m, 3H), 7.64(d, J=9Hz, | phenylsulfanyl)-5-nitro-2-(4- | | |
| | 2H), 7.56(d, J=9Hz, | toluenesulfonylamino)- | | |
| | 2H), 7.36-7.43(m, 6H), | phenyl]-4- | | |
| | 6.84(s, 1H), 2.41(m, 6H) | toluenesulfonamide | | |
| 4f | (300MHz, acetone-d6): | N-[4-(2-chloro- | M−1=602(ESI−) | >96% |
| | 7.72-7.82(m, 4H), 7.60-7.68(m, | phenylsulfanyl)-5-nitro-2-(4- | | |
| | 3H), 7.31-7.42(m, 6H), | toluenesulfonylamino)- | | |
| | 6.78(s, 1H), 2.38-2.44(m, | phenyl]-4- | | |
| | 6H) | toluenesulfonamide | | |
| 4g | (300MHz, acetone-d6): 7.88(d, | N-[4-(3-chloro- | M−1=602(ESI−) | >90% |
| | J=9Hz, 1H), 7.53-7.78(m, | phenylsulfanyl)-5-nitro-2-(4- | | |
| | 6H), 7.35-7.47(m, 6H), | toluenesulfonylamino)- | | |
| | 6.90(s, 1H), 2.40(m, 6H) | phenyl]-4- | | |
| | | toluenesulfonamide | | |
| 4h | (300MHz, acetone-d6): 7.67(s, | N-[4-(4-fluoro- | M−1=586(ESI−) | >98% |
| | 1H), 7.59-7.66(m, 4H), | phenylsulfanyl)-5-nitro-2-(4- | | |
| | 7.36-7.50(m, 8H), 6.84(s, | toluenesulfonylamino)- | | |
| | 1H), 2.42(m, 6H) | phenyl]-4- | | |
| | | toluenesulfonamide | | |
| 4i | (300MHz, acetone-d6): | N-[5-nitro-2-(4- | M−1=582(ESI−) | >89% |
| | 7.59-7.67(m, 3H), 7.52(d, J=9Hz, | toluenesulfonylamino)-4-(p- | | |
| | 2H), 7.34-7.48(m, 8H), | tolylsulfanyl)-phenyl]-4- | | |
| | 6.89(s, 1H), 2.53(s, 3H), | toluenesulfonamide | | |
| | 2.41(m, 6H) | | | |
| 4j | (300MHz, acetone-d6): 7.71(s, | N-[5-nitro-2-(4- | M−1=582(ESI−) | >97% |
| | 1H), 7.47-7.67(m, 6H), | toluenesulfonylamino)-4-(o- | | |
| | 7.34-7.43(m, 4H), 7.30(d, J=9Hz, | tolylsulfanyl)-phenyl]-4- | | |
| | 2H), 6.74(s, 1H) | toluenesulfonamide | | |
| 4k | (300MHz, acetone-d6): | N-[5-nitro-2-(4- | M−1=582(ESI−) | >89% |
| | 7.51-7.64(m, 4H), 7.32-7.49(m, | toluenesulfonylamino)-4-(m- | | |
| | 9H), 7.00(s, 1H), 2.48(s, | tolylsulfanyl)-phenyl]-4- | | |
| | 3H), 2.41(s, 6H) | toluenesulfonamide | | |
| 4l | (300MHz, acetone-d6): 7.69(s, | N-[4-(2,4-dimethyl- | M−1=596(ESI−) | >97% |
| | 1H), 7.62(d, J=7.5Hz, | phenylsulfanyl)-5-nitro-2-(4- | | |
| | 2H), 7.28-7.43(m, 9H), 6.74(s, | toluenesulfonylamino)- | | |
| | 1H), 2.50(s, 1H), 2.43(m, | phenyl]-4- | | |
| | 6H), 2.20(s, 3H) | toluenesulfonamide | | |
| 4m | (300MHz, acetone-d6): 7.74(s, | N-[4-(2,6-dimethyl- | M−1=596(ESI−) | >97% |
| | 1H), 7.63(d, J=7.5Hz, | phenylsulfanyl)-5-nitro-2-(4- | | |
| | 2H), 7.27-7.53(m, 8H), 6.67(s, | toluenesulfonylamino)- | | |
| | 1H), 2.43(m, 6H), 2.27(s, | phenyl]-4- | | |
| | 6H) | toluenesulfonamide | | |
| 4n | (300MHz, acetone-d6): | N-[4-(2-isopropyl- | M−1=610(ESI−) | >90% |
| | 7.26-7.76(m, 13H), 6.83(s, | phenylsulfanyl)-5-nitro-2-(4- | | |
| | 1H), 3.35(septet, J=6Hz, | toluenesulfonylamino)- | | |
| | 1H), 2.44(s, 6H), 1.16(d, J=6Hz, | phenyl]-4- | | |
| | 6H) | toluenesulfonamide | | |
| 4o | (300MHz, acetone-d6): | N-[5-nitro-4-phenylsulfanyl- | M−1=568(ESI−) | >97% |
| | 7.54-7.63(m, 8H), 7.31-7.44(m, | 2-(4-toluenesulfonylamino)- | | |
| | 6H), 6.91(s, 1H), 2.44(m, | phenyl]-4- | | |
| | 6H) | toluenesulfonamide | | |
| 4p | (300MHz, acetone-d6): 7.82(d, | N-[4-(furan-2- | M−1=572(ESI−) | >81% |
| | J=9Hz, 2H), 7.66(d, J=7.5Hz, | ylmethylsulfanyl)-5-nitro-2- | | |
| | 2H), 7.62(s, 1H), | (4-toluenesulfonylamino)- | | |
| | 7.54-7.58(m, 2H), 7.37-7.47(m, | phenyl]-4- | | |
| | 4H), 6.40-6.45(m, 2H), | toluenesulfonamide | | |
| | 4.15(s, 2H), 2.42(s, 6H) | | | |
| 4q | (300MHz, acetone-d6): 7.78(d, | N-[4-(4-chloro- | M−1=616(ESI−) | >97% |
| | J=9Hz, 2H), 7.66(s, | benzylsulfanyl)-5-nitro-2-(4- | | |
| | 1H), 7.62(d, J=9Hz, 2H), | toluenesulfonylamino)- | | |
| | 7.38-7.53(m, 9H), 4.12(s, | phenyl]-4- | | |
| | 2H), 2.41(s, 6H) | toluenesulfonamide | | |
| 4r | (300MHz, acetone-d6): 7.84(d, | N-[4-(2-chloro- | M−1=616(ESI−) | >86% |
| | J=7.5Hz, 2H), 7.70(s, | benzylsulfanyl)-5-nitro-2-(4- | | |
| | 1H), 7.51-7.60(m, 2H), 7.36-7.48(m, | toluenesulfonylamino)- | | |
| | 7H), 4.21(s, 2H), | phenyl]-4- | | |
| | 2.42(s, 6H) | toluenesulfonamide | | |
| 4s | (300MHz, acetone-d6): 7.82(d, | N-[4-(4-methoxy- | M−1=612(ESI−) | >95% |
| | J=7.5Hz, 2H), 7.60-7.66(m, | benzylsulfanyl)-5-nitro-2-(4- | | |
| | 3H), 7.53(s, 1H), 7.34-7.47(m, | toluenesulfonylamino)- | | |
| | 6H), 6.95(d, J=7.5Hz, | phenyl]-4- | | |
| | 2H), 4.08(s, 2H), 3.81(s, | toluenesulfonamide | | |
| | 3H), 2.42(s, 6H) | | | |
| 4t | (300MHz, acetone-d6): 7.81(d, | N-[4-benzylsulfanyl-5-nitro- | M+1=584 | >94% |
| | J=9Hz, 2H), 7.66(s, | 2-(4-toluenesulfonylamino)- | (ESI+) | |
| | 1H), 7.64(d, J=9Hz, 2H), | phenyl]-4- | | |
| | 7.55(s, 1H), 7.34-7.49(m, | toluenesulfonamide | | |
| | 9H), 4.11(s, 1H), 2.45(s, 6H) | | | |
| 4u | (300MHz, acetone-d6): 8.45(s, | N-[5-(4-chloro- | M−1=450(ESI−) | >81% |
| | 1H), 7.71(d, J=9Hz, | phenylsulfanyl)-2- | | |
| | 2H), 7.63(d, J=9Hz, 2H), | methanesulfonylamino-4- | | |
| | 7.22(s, 1H), 3.20(s, 3H), | nitro-phenyl]- | | |
| | 3.01(s, 3H) | methanesulfonamide | | |
| 6 | (300MHz, acetone-d6): 7.82(d, | N-[4-chloro-5-nitro-2-(4- | M−1=494(ESI−) | >99% |
| | J=9Hz, 2H), 7.64(d, J=9Hz, | toluenesulfonylamino)- | | |
| | 2H), 7.57(s, 1H), 7.53(s, | phenyl]-4- | | |
| | 1H), 7.45(d, J=9Hz, | toluenesulfonamide | | |
| | 2H), 7.42(d, J=9Hz, 2H), | | | |
| | 2.41(s, 6H) | | | |
| 8 | (300MHz, acetone-d6): 7.73(m, | N-[5-nitro-4-phenyl-2-(4- | M−1=536(ESI−) | >91% |
| | 4H), 7.63(s, 1H), 7.37-7.47(m, | toluenesulfonylamino)- | | |
| | 7H), 7.17(s, 1H), | phenyl]-4- | | |
| | 7.08-7.14(m, 2H), 2.43(s, | toluenesulfonamide | | |
| | 6H) | | | |
| 9a | RR | N-[4-(morpholin-4-yl)-5-nitro- | RR | RR |
| | | 2-(4-toluenesulfonylamino)- | | |
| | | phenyl]-4- | | |
| | | toluenesulfonamide | | |
| 9b | RR | N-[4-(4-methyl-piperazin-1- | RR | RR |
| | | yl)-5-nitro-2-(4- | | |
| | | toluenesulfonylamino)- | | |
| | | phenyl]-4- | | |
| | | toluenesulfonamide | | |
| 9c | RR | N-[5-nitro-4-(thiomorpholin- | RR | RR |
| | | 4-yl)-2-(4- | | |
| | | toluenesulfonylamino)- | | |
| | | phenyl]-4- | | |
| | | toluenesulfonamide | | |
| 9d | RR | N-[5-nitro-4-[(pyridin-3- | RR | RR |
| | | ylmethyl)-amino]-2-(4- | | |
| | | toluenesulfonylamino)- | | |
| | | phenyl]-4- | | |
| | | toluenesulfonamide | | |
| 9e | RR | N-[5-nitro-4-[(pyridin-2- | RR | RR |
| | | ylmethyl)-amino]-2-(4- | | |
| | | toluenesulfonylamino)- | | |
| | | phenyl]-4- | | |
| | | toluenesulfonamide | | |
| 9f | RR | N-[4-(4-methoxy- | RR | RR |
| | | benzylamino)-5-nitro-2-(4- | | |
| | | toluenesulfonylamino)- | | |
| | | phenyl]-4- | | |
| | | toluenesulfonamide | | |
| 9g | RR | N-[4-(2-chloro-benzylamino)- | RR | RR |
| | | 5-nitro-2-(4- | | |
| | | toluenesulfonylamino)- | | |
| | | phenyl]-4- | | |
| | | toluenesulfonamide | | |
| 9h | RR | N-[4-(cyclohexylmethyl- | RR | RR |
| | | amino)-5-nitro-2-(4- | | |
| | | toluenesulfonylamino)- | | |
| | | phenyl]-4- | | |
| | | toluenesulfonamide | | |
| 9i | RR | N-[4-(1-hydroxy- | RR | RR |
| | | cyclohexylmethyl-amino)-5- | | |
| | | nitro-2-(4- | | |
| | | toluenesulfonylamino)- | | |
| | | phenyl]-4- | | |
| | | toluenesulfonamide | | |
| 9j | RR | N-[4-cyclohexylamino-5- | RR | RR |
| | | nitro-2-(4- | | |
| | | toluenesulfonylamino)- | | |
| | | phenyl]-4- | | |
| | | toluenesulfonamide | | |
| 9k | RR | N-[4-[3-(5-methyl-1H- | RR | RR |
| | | pyrazol-4-yl)-propylamino]-5- | | |
| | | nitro-2-(4- | | |
| | | toluenesulfonylamino)- | | |
| | | phenyl]-4- | | |
| | | toluenesulfonamide | | |
| 2 | (300MHz, acetone-d6): 7.73(d, | N-[4-fluoro-2-(4- | ND | >86% |
| | J=8Hz, 2H), 7.55(d, J=8Hz, | toluenesulfonylamino)- | | |
| | 2H), 7.33-7.41(m, 4H), | phenyl]-4- | | |
| | 7.05(dd, J=9.9Hz, J′=2.7Hz, | toluenesulfonamide | | |
| | 1H), 6.73-6.86(m, 2H), | | | |
| | 2.41(m, 6H) | | | |
| 6 | (300MHz, acetone-d6): 8.55(br s, | N-[4-chloro-2-(4- | ND | >95% |
| | 2H), 7.67(d, J=8Hz, | toluenesulfonylamino)- | | |
| | 2H), 7.60(d, J=8Hz, 2H), | phenyl]-4- | | |
| | 7.32-7.44(m, 4H), 7.17(d, J=2.5Hz, | toluenesulfonamide | | |
| | 1H), 7.07(dd, J=7Hz, | | | |
| | J′=2.5Hz, 1H), 6.97(d, | | | |
| | J=7Hz, 1H), 2.41(s, 6H) | | | |
| 12a | (300MHz, acetone-d6): 8.40(br s, | 4-ethyl-N-[2-(4-ethyl- | M−1=565(ESI−) | <70% |
| | 2H), 7.58-7.66(m, 4H), | benzenesulfonylamino)-4-(4- | | |
| | 7.34-7.41(m, 4H), 6.95(d, J=7.5Hz, | methoxy-phenoxy)-phenyl]- | | |
| | 2H), 6.79-6.88(m, | benzenesulfonamide | | |
| | 3H), 6.68(d, J=4Hz, 1H), | | | |
| | 6.56(dd, J=9Hz, J′=4Hz, | | | |
| | 1H), 3.80(s, 3H), 2.63-2.80(m, | | | |
| | 4H), 1.20-1.35(m, 6H) | | | |
| 12b | (300MHz, acetone-d6): 8.46(br s, | 4-isopropyl-N-[2-(4- | ND | >75% |
| | 2H), 7.57-7.68(m, 4H), | isopropyl- | | |
| | 7.38-7.46(m, 4H), 6.98(d, J=7.5Hz, | benzenesulfonylamino)-4-(4- | | |
| | 2H), 6.88(d, J=7.5Hz, | methoxy-phenoxy)-phenyl]- | | |
| | 2H), 6.83(s, 1H), 6.73(d, | benzenesulfonamide | | |
| | J=4Hz, 1H), 6.55(dd, J=7.5Hz, | | | |
| | J′=4Hz, 1H), 3.84(s, | | | |
| | 3H), 3.01(septet, J=6Hz, | | | |
| | 2H), 1.24-1.33(m, 12H) | | | |
| 12c | (300MHz, acetone-d6): 8.63(br s, | 4-chloro-N-[2-(4-chloro- | M−1=577(ESI−) | >74% |
| | 2H), 7.71(d, J=7.5Hz, | benzenesulfonylamino)-4-(4- | | |
| | 4H), 7.56-7.63(m, 4H), 6.94-6.99(m, | methoxy-phenoxy)-phenyl]- | | |
| | 3H), 6.87(d, J=7.5Hz, | benzenesulfonamide | | |
| | 2H), 6.71(dd, J=7.5Hz, | | | |
| | J′=3Hz, 1H), 6.57(d, J=3Hz, | | | |
| | 1H), 3.83(s, 3H) | | | |
| 12d | (300MHz, acetone-d6): 8.47(br s, | 4-fluoro-N-[2-(4-fluoro- | M−1=545(ESI−) | <70% |
| | 2H), 8.69-8.80(m, 4H), | benzenesulfonylamino)-4-(4- | | |
| | 7.26-7.37(m, 4H), 6.85-6.99(m, | methoxy-phenoxy)-phenyl]- | | |
| | 5H), 6.70(dd, J=6Hz, J′=2Hz, | benzenesulfonamide | | |
| | 1H), 6.62(d, J=2Hz, | | | |
| | 1H), 3.81(s, 3H) | | | |
| 12e | (300MHz, acetone-d6): 8.46(br s, | N-[4-(4-methoxy-phenoxy)- | M−1=693(ESI−) | <70% |
| | 2H), 7.66-7.74(m, 4H), | 2-(4-phenoxy- | | |
| | 7.46-7.53(m, 4H), 7.22-7.32(m, | benzenesulfonylamino)- | | |
| | 2H), 7.02-7.17(m, 8H), | phenyl]-4-phenoxy | | |
| | 6.88-6.98(m, 5H), 6.74-6.72(m, | benzenesulfonamide | | |
| | 2H), 3.81(s, 3H) | | | |
| 12f | (300MHz, acetone-d6): | N-[4-(4-methoxy-phenoxy)- | M−1=599(ESI−) | >93% |
| | 8.39-8.56(m, 4H), 8.10(d, J=6Hz, | 2-(3-nitro- | | |
| | 2H), 7.39(t, J=6Hz, | benzenesulfonylamino)- | | |
| | 2H), 6.99(d, J=7.5Hz, 1H), | phenyl]-3-nitro- | | |
| | 6.94(d, J=7.5Hz, 2H), 6.84(d, | benzenesulfonamide | | |
| | J=7.5Hz, 2H), 6.71(dd, | | | |
| | J=7.5Hz, J′=3Hz, 1H), | | | |
| | 6.58(d, J=3Hz, 1H), 3.83(s, | | | |
| | 3H) | | | |
| 12g | (300MHz, acetone-d6): 8.30(br s, | N-[4-(4-methoxy-phenoxy)- | M−1=537(ESI−) | >77% |
| | 2H), 7.30-7.47(br m, | 2- | | |
| | 10H), 6.98-7.15(br m, 6H), | phenylmethanesulfonylamino- | | |
| | 6.72(br m, 1H), 4.51(s, 4H), | phenyl]-C-phenyl- | | |
| | 3.83(s, 3H) | methanesulfonamide | | |
| 12h | (300MHz, acetone-d6): | naphthalene-1-sulfonic acid | M+1=611 | >87% |
| | 8.59-8.79(m, 4H), 8.19-8.28(m, | [4-(4-methoxy-phenoxy)-2- | (ESI+) | |
| | 2H), 7.97-8.12(m, 4H), | (naphthalen-1-yl- | | |
| | 7.64-7.74(m, 4H), 7.49-7.56(m, | sulfonylamino)-phenyl]- | | |
| | 2H), 6.87(d, J=8Hz, | amide | | |
| | 2H),(6.61(d, J=8Hz, 2H), | | | |
| | 6.55(d, J=7.5Hz, 1H), 6.31-6.38(m, | | | |
| | 2H), 3.80(s, 3H), | | | |
| 12i | (300MHz, acetone-d6): | naphthalene-2-sulfonic acid | M−1=609(ESI−) | >89% |
| | 8.51-8.62(m, 2H), 8.28(d, J=9Hz, | [4-(4-methoxy-phenoxy)-2- | | |
| | 2H), 7.94-8.12(m, | (naphthalen-2-yl- | | |
| | 6H), 7.62-7.82(m, 6H), 6.95(d, | sulfonylamino)-phenyl]- | | |
| | J=6Hz, 1H), 6.49-6.77(m, | amide | | |
| | 6H) | | | |
| 12j | (300MHz, acetone-d6): 9.62(s, | N-[2-(4-acetamido- | M−1=623(ESI−) | >97% |
| | 1H), 9.57.(s, 1H), 8.37(s, | benzenesulfonylamino)-4-(4- | | |
| | 1H), 8.34(s, 1H), 7.74-7.81(m, | methoxy-phenoxy)-phenyl]- | | |
| | 4H), 7.59(d, J=9Hz, | 4-acetamido- | | |
| | 4H), 6.92-6.97(m, 3H), 6.83(d, | benzenesulfonamide | | |
| | J=9Hz, 2H), 6.60-6.66(m, | | | |
| | 2H), 3.81(s, 3H), 2.17(s, | | | |
| | 3H), 2.12(s, 3H) | | | |
| 12k | (300MHz, acetone-d6): 8.32(s, | N-[2-(4-methoxy- | M−1=569(ESI−) | >82% |
| | 2H), 7.59-7.66(m, 4H), | benzenesulfonylamino)-4-(4- | | |
| | 7.02-7.08(m, 4H), 6.95(d, J=9Hz, | methoxy-phenoxy)-phenyl]- | | |
| | 2H), 6.82-6.92(m, | 4-methoxy- | | |
| | 3H), 6.58-6.64(m, 2H), 3.91(s, | benzenesulfonamide | | |
| | 3H), 3.88(s, 3H), 3.82(s, | | | |
| | 3H) | | | |
| 12l | (300MHz, acetone-d6): 8.28(s, | butane-1-sulfonic acid [2- | M−1=469(ESI−) | ND |
| | 2H), 7.44(d, J=8Hz, | (butane-1-sulfonylamino)-4- | | |
| | 1H), 7.16(d, J=2.8Hz, 1H), | (4-methoxy-phenoxy)- | | |
| | 7.07(d, J=9Hz, 2H), 7.00(d, | phenyl]-amide | | |
| | J=9Hz, 2H), 6.79(dd, J=7.5Hz, | | | |
| | J′=2.8Hz, 1H), | | | |
| | 3.83(s, 3H), 3.10-3.20(m, | | | |
| | 4H), 1/73-1.90(m, 4H), 1.40-1.54(m, | | | |
| | 4H), 0.87-1.02(m, | | | |
| | 6H) | | | |
| 12m | (300MHz, aceteone-d6): | N-[2-(3,4-dimethoxy- | M−1=629(ESI−) | >95% |
| | 8.32(br s, 2H), 7.18-7.28(m, | benzenesulfonylamino)-4-(4- | | |
| | 3H), 7.14(d, J=1.9Hz, 1H), | methoxy-phenoxy)-phenyl]- | | |
| | 6.90-7.05(m, 5H), 6.79(d, J=8Hz, | 3,4-dimethoxy- | | |
| | 2H), 6.66(dd, J=8Hz, | benzenesulfonamide | | |
| | J′=2.5Hz, 1H), 6.53(d, | | | |
| | J=2.5Hz, 1H), 3.90(s, 3H), | | | |
| | 3.82(s, 3H), 3.78(s, 3H), | | | |
| | 3.73(s, 6H) | | | |
| 12n | (300MHz, acetone-d6): 8.46(br s, | N-[2-benzenesulfonylamino- | M−1=509(ESI−) | >96% |
| | 2H), 7.62-7.75(m, 6H), | 4-(4-methoxy-phenoxy)- | | |
| | 7.51-7.61(m, 4H), 6.96(d, J=8Hz, | phenyl]-benzenesulfonamide | | |
| | 2H), 6.80-6.88(m, | | | |
| | 3H), 6.65(d, J=3.1Hz, 1H), | | | |
| | 6.60(dd, J=8Hz, J′=3.1Hz, | | | |
| | 1H), | | | |
| 12o | (300MHz, acetone-d6): 8.45(br s, | N-[2-(4-t-butyl- | M−1=621(ESI−) | <70% |
| | 2H), 7.54-7.69(m, 8H), | benzenesulfonylamino)-4-(4- | | |
| | 6.97(d, J=8Hz, 2H), 6.80-6.89(m, | methoxy-phenoxy)-phenyl]- | | |
| | 3H), 6.76(d, J=3Hz, | 4-t-butyl- | | |
| | 1H), 6.53(dd, J=7.5Hz, | benzenesulfonamide | | |
| | J′=3Hz, 1H), 3.83(s, 3H), | | | |
| | 1.35(s, 3H), 1.32(s, 3H) | | | |
| 12p | (300MHz, acetone-d6): 8.36(br s, | N-[4-(4-methoxy-phenoxy)- | M+1=539 | >76% |
| | 2H), 7.52-7.62(m, 4H), | 2-(4-toluenesulfonylamino)- | (ESI+) | |
| | 7.31-7.40(m, 4H), 6.96(d, J=8.5Hz, | phenyl]-4- | | |
| | 2H), 6.80-6.89(m, | toluenesulfonamide | | |
| | 3H), 6.66(d, J=2.5Hz, 1H), | | | |
| | 6.58(dd, J=8Hz, J′=2.5Hz, | | | |
| | 1H), 3.82(s, 3H), 2.41(s, | | | |
| | 3H), 2.38(s, 3H) | | | |
| 12q | (300MHz, acetone-d6): 8.44(br s, | N-[4-(4-methoxy-phenoxy)- | M+1=595 | >90% |
| | 2H), 7.03(s, 2H), 6.99(s, | 2-(2,4,6- | (ESI+) | |
| | 2H), 6.93(d, J=7.5Hz, | trimethylbenzenesulfonylamino)- | | |
| | 2H), 6.74-6.82(m, 3H), 6.58(dd, | phenyl]-2,4,6- | | |
| | J=7.5Hz, J′=2.8Hz, | trimethylbenzenesulfonamide | | |
| | 1H), 6.48(d, J=2.8Hz, 1H), | | | |
| | 3.85(s, 3H), 2.48(m, 12H), | | | |
| | 2.32(s, 3H), 2.28(s, 3H) | | | |
| 12r | (300MHz, acetone-d6): | N-[4-(4-methoxy-phenoxy)- | M+1=599 ESI+ | <70% |
| | 8.32-8.43(m, 4H), 7.91-8.01(m, | 2-(4- | | |
| | 4H), 6.83-7.14(m, 8H), | nitrobenzenesulfonylamino)- | | |
| | 6.68(dd, J=8Hz, J′=2.8Hz, | phenyl]-4- | | |
| | 1H), 6.58(d, J=2.8Hz, | nitrobenzenesulfonamide | | |
| | 1H), 3.82(s, 3H), | | | |
| 12s | (300MHz, acetone-d6): 8.36(s, | N-[4-(5-hydroxy-pentyloxy)- | M+1=519 | ND |
| | 1H), 8.22(s, 1H), 8.66(d, | 2-(4-toluenesulfonylamino)- | (ESI+) | |
| | J=7.5Hz, 2H), 7.50(d, J=7.5Hz, | phenyl]-4- | | |
| | 2H), 7.24-7.37(m, | toluenesulfonamide | | |
| | 4H), 7.79(d, J=2Hz, 1H), | | | |
| | 6.63(d, J=6Hz, 1H), 6.50(dd, | | | |
| | J=6Hz, J′=2Hz, 1H), | | | |
| | 3.83(t, J=6Hz, 2H), 3.60(m, | | | |
| | 2H), 2.40(s, 6H), 1.65-1.77(m, | | | |
| | 2H), 1.42-1.62(m, | | | |
| | 4H) | | | |
| 12t | (300MHz, acetone-d6): 8.48(br s, | N-[4-(4-fluoro-phenoxy)-2- | M−1=525(ESI−) | <70% |
| | 2H), 7.56-7.64(m, 4H), | (4-toluenesulfonylamino)- | | |
| | 7.33-7.40(m, 4H), 7.15-7.22(m, | phenyl]-4- | | |
| | 2H), 6.88-6.95(m, 3H), | toluenesulfonamide | | |
| | 6.64-6.71(m, 2H), 2.41(m, | | | |
| | 6H) | | | |
| 12u | (300MHz, acetone-d6): 8.57(br s, | N-[4-(naphthalene-2-yloxy)- | M−1=557(ESI−) | <70% |
| | 2H), 7.74-8.02(m, 5H), | 2-(4-toluenesulfonylamino)- | | |
| | 7.20-7.65(m, 10H), 7.12(dd, | phenyl]-4- | | |
| | J=8Hz, J′=2Hz, 1H), 6.96(d, | toluenesulfonamide | | |
| | J=8Hz, 1H), 6.63-6.70(m, | | | |
| | 1H), 2.43(s, 3H), 2.37(s, | | | |
| | 3H) | | | |
| 12v | (300MHz, acetone-d6): 8.43(br s, | N-[4-methoxy-2-(4- | M+1=447 | <70% |
| | 1H), 8.30(br s, 1H), | toluenesulfonylamino)- | (ESI+) | |
| | 7.73(d, J=9Hz, 2H), 7.55(d, | phenyl]-4- | | |
| | J=9Hz, 2H), 7.32-7.40(m, | toluenesulfonamide | | |
| | 4H), 6.84(d, J=3Hz, | | | |
| | 1H), 6.71(d, J=7.5Hz, 1H), | | | |
| | 6.53(dd, J=7.5Hz, J′=3Hz, | | | |
| | 1H), 3.67(s, 3H), 2.41(s, | | | |
| | 6H) | | | |
| 12w | (300MHz, acetone-d6): 8.47(br s, | N-[4-(4-chloro-phenoxy)-2- | M+1=543 | <70% |
| | 2H), 7.55-7.64(m, 4H), | (4-toluenesulfonylamino)- | (ESI+) | |
| | 7.32-7.44(m, 6H), 6.96(d, J=7.5Hz, | phenyl]-4- | | |
| | 1H), 6.88(d, J=8Hz, | toluenesulfonamide | | |
| | 2H), 6.66-6.73(m, 2H), | | | |
| | 2.39(m, 6H) | | | |
| 12x | (300MHz, acetone-d6): 8.50(br s, | N-[4-(3-carboxy-phenoxy)-2- | M−1=551(ESI−) | <70% |
| | 2H), 7.85(d, J=7.5Hz, | (4-toluenesulfonylamino)- | | |
| | 1H), 7.51-7.65(m, 5H), 7.46(m, | phenyl]-4- | | |
| | 1H), 7.30-7.40(m, 4H), | toluenesulfonamide | | |
| | 7.27(dd, J=7.5Hz, J′=2.5Hz, | | | |
| | 1H), 6.93(d, J=8Hz, | | | |
| | 1H), 6.81(d, J=2.8Hz, 1H), | | | |
| | 6.73(dd, J=8Hz, J′=2.8Hz, | | | |
| | 1H), 2.40(s, 3H), 2.38(s, | | | |
| | 3H) | | | |
| 12y | (300MHz, acetone-d6): | {3-[3,4-bis(4- | M−1=565(ESI−) | <70% |
| | 7.55-7.65(m, 4H), 7.29-7.38(m, | toluenesulfonylamino)- | | |
| | 5H), 7.13(d, J=7Hz, | phenoxy]-phenyl}-acetic acid | | |
| | 1H), 6.92(m, 2H), 6.80(d, J=2.5Hz, | | | |
| | 1H), 6.71(m, 1H), | | | |
| | 6.62(dd, J=8Hz, J′=2.5Hz, | | | |
| | 1H), 3.61(s, 2H), 2.41(s, | | | |
| | 6H) | | | |
| 12z | (300MHz, acetone-d6): 8.03(d, | N-[4-(3-carboxy-phenoxy)-2- | M−1=551(ESI−) | <70% |
| | J=7.8Hz, 2H), 7.56-7.66(m, | (4-toluenesulfonylamino)- | | |
| | 4H), 7.32-7.40(m, 4H), | phenyl]-4- | | |
| | 7.06(d, J=7.5Hz, 1H), 6.87(d, | toluenesulfonamide | | |
| | J=7.8Hz, 2H), 6.74-6.83(m, | | | |
| | 2H), 2.40(s, 6H) | | | |
| 12aa | (300MHz, acetone-d6): 7.58(d, | {4-[3,4-bis(4- | M−1=565(ESI−) | >75% |
| | J=8Hz, 4H), 7.28-7.40(m, | toluenesulfonylamino)- | | |
| | 6H), 6.94(d, J=7.5Hz, | phenoxy]-phenyl}-acetic acid | | |
| | 1H), 6.81(d, J=8Hz, 2H), | | | |
| | 6.62-6.71(m, 2H), 3.66(s, | | | |
| | 2H), 2.43(s, 3H), 2.40(s, 3H) | | | |
| 12bb | (300MHz, acetone-d6): 8.39(s, | N-[4-(3-hydroxy-propoxy)-2- | M+1=491 | ND |
| | 1H), 8.27(s, 1H), 7.71(d, | (4-toluenesulfonylamino)- | (ESI+) | |
| | J=8Hz, 2H), 7.55(d, J=8Hz, | phenyl]-4- | | |
| | 2H), 7.30-7.40(m, 4H), | toluenesulfonamide | | |
| | 6.84(d, J=3Hz, 1H), 6.70(d, | | | |
| | J=7Hz, 1H), 6.53(dd, J=7Hz, | | | |
| | J′=2.7Hz, 1H), 3.94(t, | | | |
| | J=6Hz, 2H), 3.66(t, J=5.7Hz, | | | |
| | 2H), 2.41(s, 6H), | | | |
| | 1.89(quintet, J=6Hz, 2H) | | | |
| 12cc | (300MHz, acetone-d6):8.32(br s, | N-[4-allyloxy-2-(4- | M+1=473(ESI+ | ND |
| | 2H), 7.71(d, J=8Hz, | toluenesulfonylamino)- | | |
| | 2H), 7.54(d, J=8Hz, 2H), | phenyl]-4- | | |
| | 7.29-7.40(m, 4H), 6.84(d, J=3Hz, | toluenesulfonamide | | |
| | 1H), 6.68(d, J=7.5Hz, | | | |
| | 1H) 6.54(dd, J=7.5Hz, | | | |
| | J′=3Hz, 1H), 5.89-6.05(m, | | | |
| | 1H), 5.33(dd, J=16.5Hz, J′ | | | |
| | 2Hz, 1H), 5.20(dd, J=12Hz, | | | |
| | J′=2Hz, 1H), 4.44(m, | | | |
| | 2H), 2.40(s, 6H) | | | |
| 12dd | (300MHz, acetone-d6): 8.40(s, | N-[4-(8-hydroxy-octyloxy)-2- | M+1=561 | ND |
| | 1H), 8.28(s, 1H), 7.72(d, | (4-toluenesulfonylamino)- | (ESI+) | |
| | J=8Hz, 2H), 7.55(d, J=8Hz, | phenyl]-4- | | |
| | 2H), 8.30-8.40(m, 4H), | toluenesulfonamide | | |
| | 6.83(d, J=3Hz, 1H), 6.68(d, | | | |
| | J=8Hz, 1H), 6.52(dd, J=8Hz, | | | |
| | J′=3Hz, 1H), 3.84(t, | | | |
| | J=6Hz, 2H), 3.48-3.61(m, | | | |
| | 3H), 2.41(s, 6H), 1.10-1.75(m, | | | |
| | 12H) | | | |
| 12ee | (300MHz, acetone-d6): 8.41(s, | 5-[3,4-bis-(4- | M+1=561 | ND |
| | 1H), 8.30(s, 1H), 7.70(d, | toluenesulfonylamino)- | (ESI+) | |
| | J=8.5Hz, 2H), 7.55(d, J=8.5Hz, | phenoxy]-pentyl acetate | | |
| | 2H), 7.29-7.40(m, | | | |
| | 4H), 6.83(d, J=2.8Hz, 1H), | | | |
| | 6.69(d, J=8Hz, 1H), 6.53(dd, | | | |
| | J=8Hz, J′=2.8Hz, | | | |
| | 1H), 4.04(t, J=6Hz, 2H), | | | |
| | 3.85(t, J=6Hz, 2H), 2.38(s, | | | |
| | 6H), 2.00(s, 3H), 1.59-1.81(m, | | | |
| | 4H), 1.41-1.55(m, | | | |
| | 2H) | | | |
| 12ff | (300MHz, acetone-d6): 8.55(br s, | N-[4-(4-chloro- | M−1=557(ESI−) | >83% |
| | 1H), 8.48(br s, 1H), | phenylsulfanyl)-2-(4- | | |
| | 7.65(d, J=8Hz, 2H), 7.54(d, | toluenesulfonylamino)- | | |
| | J=8Hz, 2H), 7.30-7.44(m, | phenyl]-4- | | |
| | 6H), 7.05-7.21(m, 4H), | toluenesulfonamide | | |
| | 6.88(d, J=2Hz, 1H), 2.42(s, | | | |
| | 6H) | | | |
| 12gg | (300MHz, acetone-d6): 8.43(br s, | N-[4-(4-methoxy- | M+1=555 | >70% |
| | 2H), 7.60(d, J=8.5Hz, | phenylsulfanyl)-2-(4- | (ESI+) | |
| | 2H), 7.53(d, J=8.5Hz, 2H), | toluenesulfonylamino)- | | |
| | 7.24-7.38(m, 6H), 7.03(d, J=8.5Hz, | phenyl]-4- | | |
| | 2H), 6.93(d, J=8Hz, | toluenesulfonamide | | |
| | 1H), 6.83(dd, J=8Hz; J′=2.5Hz, | | | |
| | 1H), 6.74(d, J=2.5Hz, | | | |
| | 1H), 3.86(s, 3H), 2.41(s, | | | |
| | 3H), 2.37(s, 3H) | | | |
| 12hh | (300MHz, acetone-d6): | N-[4-butylsulfanyl-2-(4- | M−1=503(ESI−) | >87% |
| | 7.56-7.68(m, 4H), 7.30-7.42(m, | toluenesulfonylamino)- | | |
| | 4H), 6.90-7.02(m, 3H), | phenyl]-4- | | |
| | 2.75(m, 4H), 2.37(m, 6H), | toluenesulfonamide | | |
| | 1.27-1.54(m, 8H), 0.91(t, J=6Hz, | | | |
| | 6H) | | | |
| 15a | (300MHz, acetone-d6): 8.07(s, | N-[3-allyl-4-(4-methoxy- | ND | ND |
| | 2H), 7.43-7.53(m, 4H), | benzyloxy)-2-(4- | | |
| | 7.30-7.37(m, 4H), 7.26(d, J=7.5Hz, | toluenesulfonylamino)- | | |
| | 2H), 7.10(d, J=7.5Hz, | phenyl]-4- | | |
| | 1H), 6.99(d, J=7.5Hz, | toluenesulfonamide | | |
| | 1H), 6.94(d, J=7.5Hz, 2H), | | | |
| | 5.53-5.68(m, 1H), 5.01(s, | | | |
| | 2H), 4.78(dd, J=12Hz, J′=2Hz, | | | |
| | 1H), 4.05(dd, J=15Hz, | | | |
| | J′=2Hz, 1H), 3.80(s, | | | |
| | 3H), 2.95-3.02(m, 2H), 2.40(s, | | | |
| | 3H), 2.36(s, 3H) | | | |
| 15b | (300MHz, acetone-d6): 8.41(s, | N-[4-(4-chloro-benzyloxy)-2- | M−1=555(ESI−) | ND |
| | 1H), 8.29(s, 1H), 7.66(d, | (4-toluenesulfonylamino)- | | |
| | J=7.5Hz, 2H), 7.54(d, J=7.5Hz, | phenyl]-4- | | |
| | 2H), 7.43(m, 4H), | toluenesulfonamide | | |
| | 7.28-7.37(m, 4H), 6.95(d, J=3Hz, | | | |
| | 1H), 6.68(d, J=7.5Hz, | | | |
| | 1H), 6.60(dd, J=7.5Hz, | | | |
| | J′=3Hz, 1H), 5.01(s, 2H), | | | |
| | 2.42(m, 6H) | | | |
| 15c | (300MHz, acetone-d6): 8.42(s, | N-[4-(3-chloro-benzyloxy)-2- | M−1=555(ESI−) | ND |
| | 1H), 8.30(s, 1H), 7.67(d, | (4-toluenesulfonylamino)- | | |
| | J=7.8Hz, 2H), 7.53(d, J=7.5Hz, | phenyl]-4- | | |
| | 2H), 7.30-7.48(m, | toluenesulfonamide | | |
| | 8H), 6.96(d, J=2.6Hz, 1H), | | | |
| | 6.68(d, J=7.5Hz, 1H), 6.61(dd, | | | |
| | J=7.5Hz, J′=2.6Hz, | | | |
| | 1H), 5.02(s, 2H), 2.39(m, | | | |
| | 6H) | | | |
| 15d | (300MHz, acetone-d6): 8.43(s, | N-[4-(2-chloro-benzyloxy)-2- | M−1=555(ESI−) | <70% |
| | 1H), 8.32(s, 1H), 7.69(d, | (4-toluenesulfonylamino)- | | |
| | J=8Hz, 2H), 7.27-7.59(m, | phenyl]-4- | | |
| | 9H), 6.91(d, J=3Hz, 1H), | toluenesulfonamide | | |
| | 6.76(d, J=8Hz, 1H), 6.65(dd, | | | |
| | J=8Hz, J′=3Hz, 1H), | | | |
| | 5.05(s, 2H), 2.39(m, 6H) | | | |
| 15e | (300MHz, acetone-d6): 8.38(s, | N-[4-(4-methoxy-benzyloxy)- | M−1=551(ESI−) | >73% |
| | 1H), 8.27(s, 1H), 7.67(d, | 2-(4-toluenesulfonylamino)- | | |
| | J=8Hz, 2H), 7.53(d, J=8Hz, | phenyl]-4- | | |
| | 2H), 7.28-7.38(m, 6H), | toluenesulfonamide | | |
| | 7.39-7.49(m, 3H), 6.67(d, J=7.5Hz, | | | |
| | 1H), 6.58(dd, J=7.5Hz, | | | |
| | J′=2.5Hz, 1H), 4.90(s, | | | |
| | 2H), 3.82(s, 3H), 2.39(m, | | | |
| | 6H) | | | |
| 15f | (300MHz, acetone-d6): 8.42(s, | N-[4-cyclohexylmethoxy-2- | M+1=529 | ND |
| | 1H), 8.30(s, 1H), 7.70(d, | (4-toluenesulfonylamino)- | (ESI+) | |
| | J=8Hz, 2H), 7.30-7.58(m, | phenyl]-4- | | |
| | 6H), 6.81(d, J=2.9Hz, 1H), | toluenesulfonamide | | |
| | 6.69(d, J=8Hz, 1H), 6.52(dd, | | | |
| | J=8Hz, J′=2.9Hz, | | | |
| | 1H), 3.64(d, J=6.5Hz, 2H), | | | |
| | 2.39(s, 6H), 1.60-1.90(m), | | | |
| | 0.98-1.38(m) | | | |
| 15g | (300MHz, acetone-d6): 8.54(br s, | N-[4-(2-chloro- | M−1=571(ESI−) | >87% |
| | 2H), 7.59-7.66(m, 4H), | benzylsulfanyl)-2-(4- | | |
| | 7.14-7.45(m, 8H), 7.08(s, | toluenesulfonylamino)- | | |
| | 1H), 6.95-6.99(m, 2H), 4.08(s, | phenyl]-4- | | |
| | 2H), 2.42(s, 3H), 2.37(s, | toluenesulfonamide | | |
| | 3H) | | | |
| 17a | (300MHz, acetone-d6): 8.61(s, | N-[4-(pyrazol-1-yl)-2-(4- | M−1=481(ESI−) | >98% |
| | 1H), 8.53(s, 1H), 8.14(d, | toluenesulfonylamino)- | | |
| | J=2Hz, 1H), 7.65-7.80(m, | phenyl]-4- | | |
| | 4H), 7.60(d, J=7.5Hz, 2H), | toluenesulfonamide | | |
| | 7.30-7.53(m, 6H), 7.01(d, J=7.5Hz, | | | |
| | 1H), 6.51(m, 1H), | | | |
| | 2.40(m, 6H) | | | |
| 17b | (300MHz, acetone-d6): 8.16(s, | N-[4-(benzimidazol-1-yl)-2- | M−1=531(ESI−) | >98% |
| | 1H), 7.60-7.70(m, 5H), | (4-toluenesulfonyamino)- | | |
| | 7.21-7.48(m, 9H), 2.42(m, | phenyl]-4- | | |
| | 6H) | toluenesulfonamide | | |
| 17c | (300MHz, acetone-d6): 8.24(s, | N-[4-dimethylamino-2-(4- | M+1=460 | >85% |
| | 1H), 8.10(s, 1H), 7.72(d, | toluenesulfonylamino)- | (ESI+) | |
| | J=7.5Hz, 2H), 7.54(d, J=7.5Hz, | phenyl]-4- | | |
| | 2H), 7.29-7.37(m, | toluenesulfonamide | | |
| | 4H), 6.57(d, J=3Hz, 1H), | | | |
| | 6.54(d, J=9Hz, 1H), 6.27(dd, | | | |
| | J=9Hz, J′=3Hz, 1H), | | | |
| | 2.38(m, 6H) | | | |
| 17d | (300MHz, acetone-d6): 8.06(br s, | N-[4-(imidazol-1-yl)-2-(4- | M+1=483 | >99% |
| | 1H), 7.72(d, J=9Hz, | toluenesulfonylamino)- | (ESI+) | |
| | 2H), 7.66(d, J=9Hz, 2H), | phenyl]-4- | | |
| | 7.28-7.42(m, 8H), 7.13(d, J=7.5Hz, | toluenesulfonamide | | |
| | 1H), 2.39(s, 6H) | | | |
| 17e | (300MHz, acetone-d6): | N-{2-(4- | M−1=653(ESI−) | >71% |
| | 7.62-7.74(m, 4H), 7.44-7.56(m, | toluenesulfonylamino)-4-[4- | | |
| | 4H), 7.27-7.37(m, 4H), | (toluene-4-sulfonyl)- | | |
| | 6.74(d, J=3Hz, 1H), 6.64(d, | piperazin-1-yl]-phenyl}-4- | | |
| | J=7.8Hz, 1H), 6.51(dd, | toluenesulfonamide | | |
| | J=7.8Hz, J′=3Hz, 1H), | | | |
| | 3.02-3.18(m, 8H), 2.44(s, | | | |
| | 3H), 2.37(s, 6H) | | | |
| 17f | (300MHz, acetone-d6): 8.33(br s, | N-[4-(morpholin-4-yl)-2-(4- | M−1=500(ESI−) | >77% |
| | 1H), 8.22(br s, 1H), | toluenesulfonylamino)- | | |
| | 7.68(d, J=9Hz, 2H), 7.55(d, | phenyl]-4- | | |
| | J=9Hz, 2H), 7.28-7.39(m, | toluenesulfonamide | | |
| | 4H), 6.74(d, J=2.5Hz, | | | |
| | 1H), 6.67(d, J=7.5Hz, 1H), | | | |
| | 6.54(dd, J=7.5Hz, J′=2.5Hz, | | | |
| | 1H), 3.67-3.75(m, 4H), | | | |
| | 2.93-3.02(m, 4H), 2.41(s, | | | |
| | 6H) | | | |
| 21 | (300MHz, acetone-d6): 8.64(br s, | N-[4-(4-chloro-phenyl)-2-(4- | M−1=525(ESI−) | >70% |
| | 2H), 7.63-7.72(m, 4H), | toluenesulfonylamino)- | | |
| | 7.44(d, J=8Hz, 2H), 7.33-7.40(m, | phenyl]-4- | | |
| | 7H), 7.17-7.24(m, | toluenesulfonamide | | |
| | 2H), 2.40(s, 3H), 2.38(s, 3H) | | | |
| 22a | (300MHz, acetone-d6): 8.57(s, | N-[3-methoxy-2-(4- | M+1=447 | ND |
| | 1H), 7.92(s, 1H), 7.79(d, | toluenesulfonylamino)- | (ESI+) | |
| | J=9Hz, 2H), 7.44(d, J=9Hz, | phenyl]-4- | | |
| | 2H), 7.34(d, J=9Hz, | toluenesulfonamide | | |
| | 2H), 7.28(d, J=9Hz, 2H), | | | |
| | 7.11-7.22(m, 2H), 6.53(dd, J=7Hz, | | | |
| | J′=2.5Hz, 1H), 3.18(s, | | | |
| | 3H), 2.44-2.51(m, 6H) | | | |
| 22b | RR | N-[2-(4-methoxy-phenyl)- | RR | RR |
| | | ethyl]-3,4-bis-(4- | | |
| | | toluenesulfonylamino)- | | |
| | | benzamide | | |
| 22c | RR | N-t-butyl-3,4-bis-(4- | RR | RR |
| | | toluenesulfonylamino)- | | |
| | | benzamide | | |
| 22d | RR | N-pyridin-3-yl-3,4-bis-(4- | RR | RR |
| | | toluenesulfonylamino)- | | |
| | | benzamide | | |
| 22e | RR | N-phenyl-3,4-bis-(4- | RR | RR |
| | | toluenesulfonylamino)- | | |
| | | benzamide | | |
| 22f | RR | N-(3-hydroxy-2,2-dimethyl- | RR | RR |
| | | propyl)-3,4-bis-(4- | | |
| | | toluenesulfonylamino)- | | |
| | | benzamide | | |
| 22g | RR | N-naphthalen-1-ylmethyl- | RR | RR |
| | | 3,4-bis-(4- | | |
| | | toluenesulfonylamino)- | | |
| | | benzamide | | |
| 22h | RR | 2-phenyl-N-[3,4-bis-(4- | RR | RR |
| | | toluenesulfonylamino)- | | |
| | | phenyl]-acetamide | | |
| 22i | RR | N-[3,4-bis-(4- | RR | RR |
| | | toluenesulfonylamino)- | | |
| | | phenyl]-thiophene-2- | | |
| | | sulfonamide | | |
| 22j | RR | 3,5-dimethyl-N-[3,4-bis-(4- | RR | RR |
| | | toluenesulfonylamino)- | | |
| | | phenyl]-isoxazole-4- | | |
| | | sulfonamide | | |
| 22k | RR | 3,4-bis-(4- | RR | RR |
| | | toluenesulfonylamino)- | | |
| | | benzoic acid | | |
| 22l | RR | N-[4-hydroxymethyl-2-(4- | RR | RR |
| | | toluenesulfonylamino- | | |
| | | phenyl]-4- | | |
| | | toluenesulfonamide | | |
| 22m | RR | N-[2-methanesulfonylamino- | RR | RR |
| | | 4-(4-methoxy-phenoxy)- | | |
| | | phenyl]-4- | | |
| | | toluenesulfonamide | | |
| 22n | RR | 3,5-dimethyl-N-[5-(4- | RR | RR |
| | | methoxy-phenoxy)-2-(4- | | |
| | | toluenesulfonylamino)- | | |
| | | phenyl]-isoxazole-4- | | |
| | | sulfonamide | | |
| 22o | RR | N-[5-(4-methoxy-phenoxy)- | RR | RR |
| | | 2-(4-toluenesulfonylamino)- | | |
| | | phenyl]-1-methyl-1H- | | |
| | | imidazole-4-sulfonamide | | |
| 22p | RR | N-[2-methanesulfonylamino- | RR | RR |
| | | 5-(4-methoxy-phenoxy)- | | |
| | | phenyl]-4- | | |
| | | toluenesulfonamide | | |
| 22q | RR | N-{5-[4-(4-methoxy- | RR | RR |
| | | phenoxy)-2-(4- | | |
| | | toluenesulfonylamino)- | | |
| | | phenylsulfamoyl]-4-methyl- | | |
| | | thiazol-2-yl}-acetamide | | |
| 22r | RR | 3,5-dimethyl-N-[4-(4- | RR | RR |
| | | methoxy-phenoxy)-2-(4- | | |
| | | toluenesulfonylamino)- | | |
| | | phenyl]-isoxazole-4- | | |
| | | sulfonamide | | |
| 22s | RR | N-[4-(4-methoxy-phenoxy)- | RR | RR |
| | | 2-(4-toluenesulfonylamino)- | | |
| | | phenyl]-1-methyl-1H- | | |
| | | imidazole-4-sulfonamide | | |
| 22t | RR | 5-bromo-6-chloro-N-[4-(4- | RR | RR |
| | | methoxy-phenoxy)-2-(4- | | |
| | | toluenesulfonylamino)- | | |
| | | phenyl]-pyridine-3- | | |
| | | sulfonamide | | |
| 22u | RR | 7-chloro-N-[4-(4-methoxy- | RR | RR |
| | | phenoxy)-2-(4- | | |
| | | toluenesulfonylamino)- | | |
| | | phenyl]- | | |
| | | benzo[1,2,5]oxadiazole-4- | | |
| | | sulfonamide | | |
| 22v | RR | 5-isoxazol-3-yl-N-[4-(4- | RR | RR |
| | | methoxy-phenoxy)-2-(4- | | |
| | | toluenesulfonylamino)- | | |
| | | phenyl]-thiophene-2- | | |
| | | sulfonamide | | |
| 22w | RR | methyl 4-[4-(4-methoxy- | RR | RR |
| | | phenoxy)-2-(4- | | |
| | | toluenesulfonylamino)- | | |
| | | phenylsulfamoyl]-1,2,5- | | |
| | | trimethyl-1H-pyrrole-3- | | |
| | | carboxylate | | |
| 22x | RR | 4-butyl-N-[4-(4-methoxy- | RR | RR |
| | | phenoxy)-2-(4- | | |
| | | toluenesulfonylamino)- | | |
| | | phenyl]-benzenesulfonamide | | |
| 22y | RR | N-[5-(4-methoxy-phenoxy)- | RR | RR |
| | | 2-(2-naphthalen-1-yl- | | |
| | | ethanesulfonylamino)- | | |
| | | phenyl]-4- | | |
| | | toluenesulfonamide | | |
| 22z | RR | 4-methanesulfonyl-N-[4-(4- | RR | RR |
| | | methoxy-phenoxy)-2-(4- | | |
| | | toluenesulfonylamino)- | | |
| | | phenyl]-benzenesulfonamide | | |
| 22aa | RR | 3-methoxy-N-[4-(4-methoxy- | RR | RR |
| | | phenoxy)-2-(4- | | |
| | | toluenesulfonylamino)- | | |
| | | phenyl]-benzenesulfonamide | | |
| 22bb | RR | N-[4-(4-methoxy-phenoxy)- | RR | RR |
| | | 2-(4-toluenesulfonylamino)- | | |
| | | phenyl]-5-bromo-thiophene- | | |
| | | 2-sulfonamide | | |
| 22cc | RR | N-[4-(4-methoxy-phenoxy)- | RR | RR |
| | | 2-(4-toluenesulfonylamino)- | | |
| | | phenyl]-isoquinoline-5- | | |
| | | sulfonamide | | |
| 22dd | RR | methyl 4-[5-(4-methoxy- | RR | RR |
| | | phenoxy)-2-(4- | | |
| | | toluenesulfonylamino)- | | |
| | | phenylsulfamoyl]-1,2,5- | | |
| | | trimethyl-1H-pyrrole-3- | | |
| | | carboxylate | | |
| 22ee | RR | N-[4-(4-methoxy-phenoxy)- | RR | RR |
| | | 2-(2-naphthalen-1-yl- | | |
| | | ethanesulfonylamino)- | | |
| | | phenyl]-4- | | |
| | | toluenesulfonamide | | |
| 22ff | RR | 4-methanesulfonyl-N-[5-(4- | RR | RR |
| | | methoxy-phenoxy)-2-(4- | | |
| | | toluenesulfonylamino)- | | |
| | | phenyl]-benzenesulfonamide | | |
| 22gg | RR | 5-bromo-N-[5-(4-methoxy- | RR | RR |
| | | phenoxy)-2-(4- | | |
| | | toluenesulfonylamino)- | | |
| | | phenyl]-thiophene-2- | | |
| | | sulfonamide | | |
| 22hh | RR | 2-methoxy-N-[5-(4-methoxy- | RR | RR |
| | | phenoxy)-2-(4- | | |
| | | toluenesulfonylamino)- | | |
| | | phenyl]-4-methyl- | | |
| | | benzenesulfonamide | | |
| 22ii | RR | N-[4-(benzyloxyimino- | RR | RR |
| | | phenyl-methyl)-2-(4- | | |
| | | toluenesulfonylamino)- | | |
| | | phenyl]-4- | | |
| | | toluenesulfonamide | | |
| 22jj | RR | N-[4-(hydroxyimino-phenyl- | RR | RR |
| | | methyl)-2-(4- | | |
| | | toluenesulfonylamino)- | | |
| | | phenyl]-4- | | |
| | | toluenesulfonamide | | |
| 22kk | RR | N-[4-(methoxyimino-phenyl- | RR | RR |
| | | methyl)-2-(4- | | |
| | | toluenesulfonylamino)- | | |
| | | phenyl]-4- | | |
| | | toluenesulfonamide | | |
| 22ll | RR | N-[5-(2-chloro- | RR | RR |
| | | benzylsulfanyl)-2- | | |
| | | methanesulfonylamino- | | |
| | | phenyl]-4- | | |
| | | toluenesulfonamide | | |
| 22mm | RR | N-[4-(2-chloro- | RR | RR |
| | | benzylsulfanyl)-2-(4- | | |
| | | toluenesulfonylamino)- | | |
| | | phenyl]-4- | | |
| | | toluenesulfonamide | | |
| 22nn | RR | N-[4-(2-chloro- | RR | RR |
| | | benzylsulfanyl)-2- | | |
| | | methanesulfonylamino- | | |
| | | phenyl]-4- | | |
| | | toluenesulfonamide | | |
| 23 | (300MHz, acetone-d6): 7.80(d, | N-[5-amino-4-(4-chloro- | M−1=572(ESI−) | >86% |
| | J=8.3Hz, 2H), 7.51(d, J=8.3Hz, | phenylsulfanyl)-2-(4- | | |
| | 2H), 7.38(d, J=8.3Hz, | toluenesulfonylamino)- | | |
| | 2H0, 7.24-7.32(m, 4H), | phenyl]-4- | | |
| | 7.03(s, 1H), 6.81(d, J=8Hz, | toluenesulfonamide | | |
| | 2H), 6.46(s, 1H), 2.40(s, | | | |
| | 3H), 2.25(s, 3H) | | | |
| 24 | (300MHz, acetone-d6): 7.96(d, | N-[2-(4-chloro- | ND | <70% |
| | J=8.5Hz, 2H), 7.86(d, J=8.5Hz, | phenylsulfanyl)-4,5-bis-(4- | | |
| | 2H), 7.53(s, 1H), | toluenesulfonylamino)- | | |
| | 7.30-7.45(m, 8H), 7.14(s, | phenyl]-acetamide | | |
| | 1H), 2.48(s, 3H), 2.45(s, | | | |
| | 3H), 1.97(s, 3H) | | | |
| 33 | (300MHz, acetone-d6): 8.64(s, | methyl 2-(4-methoxy- | ND | ND |
| | 1H), 8.47(s, 1H), 7.50-7.43(m, | phenoxy)-4,5-bis-(4- | | |
| | 4H), 7.29-7.40(m, | toluenesulfonylamino)- | | |
| | 4H), 7.25(s, 1H), 7.04(d, J=8Hz, | benzoate | | |
| | 2H), 6.83(d, J=8Hz, | | | |
| | 2H), 6.72(s, 1H), 3.83(s, | | | |
| | 3H), 6.71(s, 3H), 2.42(s, 6H) | | | |
| 34 | (300MHz, acetone-d6): 7.60(d, | 2-(4-methoxy-phenoxy)-4,5- | M−1=581(ESI−) | ND |
| | J=7.8Hz, 2H), 7.53(d, J=7.8Hz, | bis-(4- | | |
| | 2H), 7.30-7.42(m, | toluenesulfonylamino)- | | |
| | 4H), 7.04(d, J=8Hz, 2H), | benzoic acid | | |
| | 6.87(d, J=8Hz, 2H), 6.73(s, | | | |
| | 1H), 3.89(s, 3H), 2.40(m, | | | |
| | 6H) | | | |
| 37 | (300MHz, acetone-d6): 8.28(s, | N-[4-(4-methoxy-phenoxy)- | M+1=553 | ND |
| | 1H), 8.18(s, 1H), 7.58(d, | 5-methyl-2-(4- | (ESI+) | |
| | J=8.5Hz, 2H), 7.48(d, J=8.5Hz, | toluenesulfonylamino)- | | |
| | 2H), 7.27-7.38(m, | phenyl]-4- | | |
| | 4H), 6.87-6.96(m, 3H), 6.71(d, | toluenesulfonamide | | |
| | J=9Hz, 2H), 6.32(s, | | | |
| | 1H), 3.81(s, 3H), 2.43(s, | | | |
| | 3H), 2.40(s, 3H) | | | |
| 38a | Sigma-Aldrich Rare | N-[4,5-dibromo-2-(4- | Aldrich | Aldrich |
| | Chemicals Library | toluenesulfonylamino)- | | |
| | | phenyl]-4- | | |
| | | toluenesulfonamide | | |
| 38b | RR | N-[4-(4-methoxy-phenoxy)- | RR | RR |
| | | 2-(4-toluenesulfonylamino)- | | |
| | | 5-trifluoromethyl-phenyl]-4- | | |
| | | toluenesulfonamide | | |
| 38c | RR | N-[4-(2-chloro- | RR | RR |
| | | benzylsulfanyl)-5-fluoro-2-(4- | | |
| | | toluenesulfonylamino)- | | |
| | | phenyl]-4- | | |
| | | toluenesulfonamide | | |
| 38d | RR | N-(4-(2-chloro- | RR | RR |
| | | phenylmethanesulfonyl)-5- | | |
| | | fluoro-2-(4- | | |
| | | toluenesulfonylamino)- | | |
| | | phenyl]-4- | | |
| | | toluenesulfonamide | | |
| 42 | (300MHz, acetone-d6): 7.61(d, | diethyl-5-(4-methoxy- | ND | >86% |
| | J=8Hz, 2H), 7.44(d, J=7.8Hz, | phenoxy)-2-(4- | | |
| | 1H), 7.36(d, J=8Hz, | toluenesulfonylamino)- | | |
| | 2H), 7.01(m, 4H), 6.74-6.83(m, | phenyl phosphate | | |
| | 2H), 3.96-4.11(m, | | | |
| | 4H), 3.80(s, 3H), 2.40(s, | | | |
| | 3H), 1.21(t, J=6Hz, 6H) | | | |
| 43 | (300MHz, acetone-d6): 7.66(d, | [5-(4-methoxy-phenoxy)-2- | M−1=464 | ND |
| | J=8.5Hz, 2H), 7.44(d, J=8Hz, | (4-toluenesulfonylamino)- | (ESI−) | |
| | 1H), 7.29(d, J=8.5Hz, | phenyl]-monophosphate | | |
| | 2H), 6.96(m, 4H), 6.68(m, | | | |
| | 2H), 3.79(s, 3H), 2.36(s, | | | |
| | 3H) | | | |
| 48 | (300MHz, acetone-d6): | 4-(4-methoxy- | M+1=555 | ND |
| | 7.60-7.70(m, 4H), 7.38-7.49(m, | phenoxymethyl)-2-(4- | (ESI+) | |
| | 5H), 7.25-7.36(m, 2H), | toluenesulfonyloxy)-phenyl | | |
| | 6.95(d, J=8Hz, 2H), 6.88(d, | 4-toluenesulfonate | | |
| | J=8Hz, 2H), 5.10(s, | | | |
| | 2H), 3.77(s, 3H), 2.47(m, | | | |
| | 6H) | | | |
| 51 | (300MHz, acetone-d6): 9.90(br m, | N-[4-(4-chloro- | M−1=530(ESI−) | >92% |
| | 2H), 8.72(s, 1H), 7.96(d, | phenylsulfanyl)-5-nitro-2-(4- | | |
| | J=7.8Hz, 2H), 7.78(d, J=7.8Hz, | toluamido)-phenyl]-4- | | |
| | 2H), 7.64-7.73(m, | toluamide | | |
| | 3H), 7.60(d, J=7.8Hz, 2H), | | | |
| | 7.28-7.38(m, 4H), 2.40(s, | | | |
| | 3H), 2.35(s, 3H) | | | |
| 52 | (300MHz, acetone-d6): 9.85(br s, | N-[4-(4-chloro- | M+1=487 | ND |
| | 1H), 7.91-7.97(m, 6H), | phenylsulfanyl)-2-(4- | (ESI+) | |
| | 7.30-7.44(m, 9H), 2.42(m, | toluoylamino)-phenyl]-4- | | |
| | 6H) | toluamide | | |
| 53 | (300MHz, acetone-d6): | N-[4-(4-chloro- | M+1=632 | >81% |
| | 7.66-7.74(m, 3H), 7.45-7.56(m, | phenylsulfanyl)-5-nitro-2-(4- | (ESI+) | |
| | 10H), 6.75(s, 1H), 3.22(s, | toluenesulfonyl-methyl- | | |
| | 3H), 2.94(s, 3H), 2.61(s, | amino)-phenyl]-N-methyl-4- | | |
| | 3H), 2.50(s. 3H) | toluenesulfonamide | | |
| 55 | (300MHz, acetone-d6): 8.34(br s, | N-[2-amino-4-(4-chloro- | M+1=450 | >85% |
| | 1H), 7.70(d, J=7.5Hz, | phenylsulfanyl)-5-nitro- | | |
| | 2H), 7.61(m, 4H), 7.41(d, J=7.5Hz, | phenyl]-4- | | |
| | 2H), 6.15(s, 1H), | toluenesulfonamide | | |
| | 6.08(br s, 2H), 2.44(s, 3H) | | | |
| 56 | (300MHz, acetone-d6): 9.11(s, | N-[2-amino-4-(4-chloro- | M−1=412(ESI−) | >93% |
| | 1H), 8.38(s, 1H), 7.93(d, | phenylsulfanyl)-5-nitro- | | |
| | J=7Hz, 2H), 7.68(d, J=7.8Hz, | phenyl]-4-toluamide | | |
| | 2H), 7.59(d, J=7.8Hz, | | | |
| | 2H), 7.33(d, J=7.5Hz, 2H), | | | |
| | 6.73(s, 1H), 2.42(s, 3H) | | | |
| 59 | (300MHz, acetone-d6): 7.66(d, | N-[2-hydroxy-4-(4-methoxy- | M+1=386 | ND |
| | J=8Hz, 2H), 7.32(d, J=8Hz, | phenoxy)-phenyl]-4- | (ESI+) | |
| | 2H), 7.14(d, J=8.2Hz, | toluenesulfonamide | | |
| | 1H), 6.95(m, 4H), 6.30-6.39(m, | | | |
| | 2H), 3.79(s, 3H), | | | |
| | 2.39(s, 3H) | | | |
| 60 | (300MHz, acetone-d6): 7.95(s, | N-[2-methoxy-4-(4-methoxy- | ND | ND |
| | 1H), 7.62(d, J=8Hz, | phenoxy)-phenyl]-4- | | |
| | 2H), 7.25-7.38 m, 4H), 6.95(m, | toluenesulfonamide | | |
| | 4H), 6.50(d, J=2.8Hz, | | | |
| | 1H), 6.40(dd, J=7.5Hz, J′=2.8Hz, | | | |
| | 1H), 3.80(s, 3H), | | | |
| | 3.50(s, 3H), 2.40(s, 3H) | | | |
| 61 | (300MHz, acetone-d6): 7.57(d, | N-[2-methoxy-4-(4-methoxy- | M+1=414 | ND |
| | J=8Hz, 2H), 7.38(d, J=8Hz, | phenoxy)-phenyl]-N-methyl- | (ESI+) | |
| | 2H), 7.15(d, J=7.8Hz, | 4-toluenesulfonamide | | |
| | 2H), 7.04(d, J=7.5Hz, | | | |
| | 2H), 6.98(d, J=7.5Hz, 2H), | | | |
| | 6.55(d, J=3Hz, 1H), 6.37(dd, | | | |
| | J=7.8Hz, J′=3Hz, | | | |
| | 1H), 3.82(s, 3H), 3.37(s, | | | |
| | 3H), 3.16(s, 3H), 2.45(s, 3H) | | | |
| 62 | (300MHz, acetone-d6): 8.16(s, | N-[2-(2-hydroxy-ethoxy)-4- | M−1=428(ESI−) | ND |
| | 1H), 7.62(d, J=7.5Hz, | (4-methoxy-phenoxy)- | | |
| | 2H), 7.42(d, J=8Hz, 1H), | phenyl]-4- | | |
| | 7.32(d, J=7.5Hz, 2H), 6.97(m, | toluenesulfonamide | | |
| | 4H), 6.53(d, J=2.5Hz, | | | |
| | 1H), 6.46(dd, J=8Hz, J′=2.5Hz, | | | |
| | 1H), 4.17(t, J=6.2Hz, | | | |
| | 1H), 3.81(s, 3H), 3.74(t, | | | |
| | J=6Hz, 2H), 3.68(m, 2H), | | | |
| | 2.38(s, 3H) | | | |
| 63 | (300MHz, acetone-d6): 8.05(s, | N-[2-(3-hydroxy-propoxy)-4- | M+1=444 | ND |
| | 1H), 7.61(d, J=7.8Hz, | (4-methoxy-phenoxy)- | (ESI+) | |
| | 2H), 7.41(d, J=8.5Hz, 1H), | phenyl]-4- | | |
| | 7.32(d, J=7.8Hz, 2H), 6.96(m, | toluenesulfonamide | | |
| | 4H), 6.53(d, J=2.3Hz, | | | |
| | 1H), 6.43(dd, J=8.5Hz, J′=2.3Hz, | | | |
| | 1H), 3.72-3.85(m, | | | |
| | 5H), 3.55-3.69(m, 3H), 2.39(s, | | | |
| | 3H), 1.79(quintet, J=5.8Hz, | | | |
| | 2H) | | | |
| 64 | (300MHz, acetone-d6): 8.20(br s, | N-[2-(2,3-dihydroxy- | M−1=458(ESI−) | ND |
| | 1H), 7.60(d, J=7.5Hz, | propoxy)-4-(4-methoxy- | | |
| | 2H), 7.42(d, J=8Hz, 1H), | phenoxy)-phenyl]-4- | | |
| | 7.32(d, J=7.5Hz, 2H), 6.96(m, | toluenesulfonamide | | |
| | 4H), 6.54(d, J=2.8Hz, | | | |
| | 1H), 6.47(dd, J=8Hz, J′=2.8Hz, | | | |
| | 1H), 4.36(br s, 1H), | | | |
| | 3.65-3.85(m, 7H), 3.54(m, | | | |
| | 2H), 2.40(s, 3H) | | | |
| 65 | (300MHz, acetone-d6): 8.28(br s, | [5-(4-methoxy-phenoxy)-2- | M+1=444 | ND |
| | 1H), 7.66(d, J=7.8Hz, | (4-toluenesulfonylamino)- | (ESI+) | |
| | 2H), 7.43(d, J=8Hz; 1H), | phenoxy]-acetic acid | | |
| | 7.30(d, J=7.8Hz, 2H), 6.96(m, | | | |
| | 4H), 6.59(d, J=2.5Hz, | | | |
| | 1H), 6.51(dd, J=8Hz, J′=2.5Hz, | | | |
| | 1H), 4.53(s, 3H), | | | |
| | 3.80(s, 3H), 2.39(s, 3H) | | | |
| 66 | (300MHz, acetone-d6): 8.09(s, | N-[2-methoxy-5-(4-methoxy- | M+1=414 | ND |
| | 1H), 7.60-7.68(m, 3H), | phenoxymethyl)-phenyl]-4- | (ESI+) | |
| | 7.28(d, J=8Hz, 2H), 7.15(d, | toluenesulfonamide | | |
| | J=8.2Hz, 1H), 6.83-6.96(m, | | | |
| | 5H), 4.99(s, 2H), 3.76(s, | | | |
| | 3H), 3.65(s, 3H), 2.37(s, 3H) | | | |
| 67 | (300MHz, acetone-d6): | methyl 5-(4-methoxy- | M+1=427 | ND |
| | 7.65-7.73(m, 3H), 7.33-7.42(m, | phenoxy)-2-(4- | (ESI+) | |
| | 3H), 7.29-7.37(m, 1H), | toluenesulfonylamino)- | | |
| | 6.97(m, 4H), 3.78-3.84(m, | benzoate | | |
| | 6H), 2.38(s, 3H) | | | |
| 68 | (300MHz, acetone-d6): | 5-(4-methoxy-phenoxy)-2-(4- | M+1=414 | ND |
| | 7.68-7.76(m, 3H), 7.49(d, J=2.8Hz, | toluenesulfonylamino)- | (ESI+) | |
| | 1H), 7.35(d, J=8Hz, | benzoic acid | | |
| | 2H), 7.22(dd, J=8Hz, J′=2.8Hz, | | | |
| | 1H), 6.97(m, 4H), | | | |
| | 6.76(s, 1H), 3.81(s, 3H), | | | |
| | 2.39(s, 3H) | | | |
| 69 | (300MHz, acetone-d6): 9.50(s, | N-[4-(4-chloro- | ND | >98% |
| | 1H), 8.12(d, J=3Hz, | phenylsulfanyl)-3-nitro- | | |
| | 1H), 7.75(d, J=7.8Hz, 2H), | phenyl]-4- | | |
| | 7.54-7.63(m, 4H), 7.34-7.44(m, | toluenesulfonamide | | |
| | 3H), 6.93(d, J=7.8Hz, | | | |
| | 1H), 2.39(s, 3H) | | | |
| 70 | (300MHz, acetone-d6): 8.12(d, | N-[3-(4-chloro- | M−1=433(ESI−) | >86% |
| | J=7.8Hz, 1H), 7.51-7.72(m, | phenylsulfanyl)-4-nitro- | | |
| | 6H), 7.40(d, J=7.5Hz, | phenyl]-4- | | |
| | 2H), 7.17(dd, J=7.8Hz, J′=2.8Hz, | toluenesulfonamide | | |
| | 1H), 6.79(d, J=2.8Hz, | | | |
| | 1H), 2.44(s, 3H) | | | |
| 71a | RR | N-[2-amino-5-(4-methoxy- | RR | RR |
| | | phenoxy)-phenyl]-4- | | |
| | | toluenesulfonamide | | |
| 71b | RR | 6-cyano-N-[4-(4-methoxy- | RR | RR |
| | | phenoxy)-2-(4- | | |
| | | toluenesulfonylamino)- | | |
| | | phenyl]-nicotinamide | | |
| 71c | RR | N-[4-(4-methoxy-phenoxy)- | RR | RR |
| | | 2-(4-toluenesulfonylamino)- | | |
| | | phenyl]-6-trifluoromethyl- | | |
| | | nicotinamide | | |
| 71d | RR | N-[2-(4-cyano-benzylamino)- | RR | RR |
| | | 5-(4-methoxy-phenoxy)- | | |
| | | phenyl]-4- | | |
| | | toluenesulfonamide | | |
| 71e | RR | N-(4-{[4-(4-methoxy- | RR | RR |
| | | phenoxy)-2-(4- | | |
| | | toluenesulfonylamino)- | | |
| | | phenylamino]-methyl}- | | |
| | | phenyl)-acetamide | | |
| 71f | RR | N-[2-[(benzofuran-2- | RR | RR |
| | | ylmethyl)-amino]-5-(4- | | |
| | | methoxy-phenoxy)-phenyl]- | | |
| | | 4-toluenesulfonamide | | |
| 71g | RR | N-{5-(4-methoxy-phenoxy)- | RR | RR |
| | | 2-[(quinolin-2-ylmethyl)- | | |
| | | amino]-phenyl}-4- | | |
| | | toluenesulfonamide | | |
| 71h | RR | N-[5-(4-methoxy-phenoxy)- | RR | RR |
| | | 2-(4-toluenesulfonylamino)- | | |
| | | phenyl]-5-methyl-pyrazine-2- | | |
| | | carboxamide | | |
| 71i | RR | N-[2-amino-5-(4-methoxy- | RR | RR |
| | | phenoxy)-benzyl]-4- | | |
| | | toluenesulfonamide | | |
| 71j | RR | N-[2-cyano-4-(4-methoxy- | RR | RR |
| | | phenoxy)-phenyl]-4- | | |
| | | toluenesulfonamide | | |
| 71k | RR | 5-(4-methoxy-phenoxy)-2-(4- | RR | RR |
| | | toluenesulfonylamino)- | | |
| | | benzamide | | |
| 71l | RR | N-[2-amino-4-(4-methoxy- | RR | RR |
| | | phenoxy)-phenyl]-4- | | |
| | | toluenesulfonamide | | |
| 71m | RR | N-[4-(4-methoxy-phenoxy)- | RR | RR |
| | | 2-(4-toluenesulfonylamino- | | |
| | | methyl)-phenyl]-4- | | |
| | | toluenesulfonamide | | |
| 71n | RR | N-[2-aminomethyl-4-(4- | RR | RR |
| | | methoxy-phenoxy)-phenyl]- | | |
| | | 4-toluenesulfonamide | | |
| 71o | RR | N-[3-[benzoyl-(4- | RR | RR |
| | | toluenesulfonyl)-amino]-4-(4- | | |
| | | toluenesulfonylamino)- | | |
| | | phenyl]-benzamide | | |
| 71p | RR | N-[5-(4-methoxy-phenoxy)- | RR | RR |
| | | 2-(4-toluenesulfonylamino)- | | |
| | | benzyl]-4-toluamide | | |
| 71q | RR | N-[5-(4-methoxy-phenoxy)- | RR | RR |
| | | 2-(4-toluenesulfonylamino)- | | |
| | | phenyl]-4-toluamide | | |
| 71r | RR | N-[4-(4-methoxy-phenoxy)- | RR | RR |
| | | 2-(4-toluenesulfonylamino)- | | |
| | | phenyl]-4-toluamide | | |
| 71s | RR | N-[4-(4-methoxy-phenoxy)- | RR | RR |
| | | 2-(4-toluenesulfonylamino)- | | |
| | | phenyl]-2-phenyl-acetamide | | |
| 71t | RR | N-[2-amino-5-(4-methoxy- | RR | RR |
| | | phenoxy)-phenyl]-N-methyl- | | |
| | | 4-toluenesulfonamide | | |
| 71u | RR | N-{4-(4-methoxy-phenoxy)- | RR | RR |
| | | 2-[(quinolin-2-ylmethylene)- | | |
| | | amino]-phenyl}-4- | | |
| | | toluenesulfonamide | | |
| 71v | RR | 4-(4-methoxy- | RR | RR |
| | | phenoxymethyl)-2-(4- | | |
| | | toluenesulfonylamino)- | | |
| | | benzamide | | |
| 71w | RR | N-[2-aminomethyl-5-(4- | RR | RR |
| | | methoxy-phenoxymethyl)- | | |
| | | phenyl]-4- | | |
| | | toluenesulfonamide | | |
| 71x | RR | N-[2- | RR | RR |
| | | (methanesulfonylamino- | | |
| | | methyl)-5-(4-methoxy- | | |
| | | phenoxymethyl)-phenyl]-6- | | |
| | | methyl-naphthalene-2- | | |
| | | sulfonamide | | |
| 71y | RR | N-(4-morpholin-4-yl-5-nitro- | RR | RR |
| | | 2-(4-toluoylamino)-phenyl)- | | |
| | | 4-toluamide | | |
| 71z | RR | N-[5-(4-methoxy-phenoxy)- | RR | RR |
| | | 2-(4-toluenesulfonylamino)- | | |
| | | phenyl]-butyramide | | |
| 71aa | RR | N-[4-(cyclohexylmethyl- | RR | RR |
| | | amino)-5-nitro-2-(4- | | |
| | | toluoylamino)-phenyl]-4- | | |
| | | toluamide | | |
| 71bb | RR | N-{4-[(naphthalen-1- | RR | RR |
| | | ylmethyl)-amino]-5-nitro-2- | | |
| | | (4-toluoylamino)-phenyl}-4- | | |
| | | toluamide | | |
| 71cc | RR | N-[2-amino-4-(4-methoxy- | RR | RR |
| | | phenoxy)-5-nitro-phenyl]-4- | | |
| | | toluenesulfonamide | | |
| 71dd | RR | N-[4-(4-methoxy-phenoxy)- | RR | RR |
| | | 2-(4-toluenesulfonyl-methyl- | | |
| | | amino)-phenyl]-4- | | |
| | | toluenesulfonamide | | |
| 71ee | RR | N-[2-amino-4-(4-methoxy- | RR | RR |
| | | phenoxy)-5-trifluoromethyl- | | |
| | | phenyl]-4- | | |
| | | toluenesulfonamide | | |
| 71ff | RR | N-[4-(4-methoxy- | RR | RR |
| | | phenoxymethyl)-2-(4- | | |
| | | toluenesulfonylamino)- | | |
| | | benzyl]-4-toluamide | | |
| 71gg | RR | N-[2-amino-5-(4-methoxy- | RR | RR |
| | | phenoxy)-4-trifluoromethyl- | | |
| | | phenyl]-4- | | |
| | | toluenesulfonamide | | |
| 71hh | RR | N-[4-(4-methoxy-phenoxy)- | RR | RR |
| | | 2-(4-methyl-benzylamino)- | | |
| | | phenyl]-4- | | |
| | | toluenesulfonamide | | |
| 71ii | RR | N-{4-(4-methoxy-phenoxy)- | RR | RR |
| | | 2-[3-(4-toluenesulfonyl)- | | |
| | | ureido]-phenyl}-4- | | |
| | | toluenesulfonamide | | |
| 71jj | RR | N-[5-(4-methoxy-phenoxy)- | RR | RR |
| | | 2-(4-methyl-benzylamino)- | | |
| | | phenyl]-4- | | |
| | | toluenesulfonamide | | |
| 71kk | RR | N-[2-amino-5-(2-chloro- | RR | RR |
| | | benzylsulfanyl)-phenyl]-4- | | |
| | | toluenesulfonamide | | |
| 71ll | RR | N-[2-amino-4-(2-chloro- | RR | RR |
| | | benzylsulfanyl)-5-nitro- | | |
| | | phenyl]-7-chloro- | | |
| | | benzo[1,2,5]oxadiazole-4- | | |
| | | sulfonamide | | |
| 71mm | RR | N-(4-ethyl-phenyl)-4- | RR | RR |
| | | methoxy-2-(4- | | |
| | | toluenesulfonylamino)- | | |
| | | benzenesulfonamide | | |
| 71nn | RR | N-[4-(2-chloro- | RR | RR |
| | | phenylmethanesulfonyl)-2- | | |
| | | (4-toluenesulfonylamino)- | | |
| | | phenyl]-4- | | |
| | | toluenesulfonamide | | |
| 72a | RR | 6-(4-methoxy-phenoxy)-1-(4- | RR | RR |
| | | toluenesulfonyl)-1,3-dihydro- | | |
| | | benzoimidazol-2-one | | |
| 72b | RR | 5-(4-methoxy-phenoxy)-1-(4- | RR | RR |
| | | toluenesulfonyl)-1,3-dihydro- | | |
| | | benzoimidazol-2-one | | |
| 72c | RR | 5-(4-methoxy-phenoxy)-1,3- | RR | RR |
| | | bis-(4-toluenesulfonyl)-1,3- | | |
| | | dihydro-benzoimidazol-2- | | |
| | | one | | |
| 72d | RR | 5-(4-methoxy-phenoxy)-1,3- | RR | RR |
| | | dihydro- | | |
| | | benzo[1,2,5]thiadiazole 2,2- | | |
| | | dioxide | | |
| 72e | RR | 5-(4-methoxy-phenoxy)-1,3- | RR | RR |
| | | dihydro-benzoimidazol-2- | | |
| | | one | | |
| 72f | RR | 5-(4-methoxy-phenoxy)-1-(4- | RR | RR |
| | | toluenesulfonyl)-1,3-dihydro- | | |
| | | benzo[1,2,5]thiadiazole 2,2- | | |
| | | dioxide | | |

### Example 16

Specificity of Sulfonamide-Based Inhibitors for Src

Recombinant human Src was expressed using the baculovirus-insect cell system and purified as published (Budde et al., 1993 and 2000). Recombinant Csk and the FGF receptor (FGFr) were expressed as glutathione-5-transferase fusion proteins using the pGEX expression vector and E. coli, and purified as described (Sun & Budde, 1995).

The tyrosine kinase activity of Src, Csk and FGFr was determined using poly E₄Y and ³²P-ATP. Briefly, enzymes were assayed in a reaction mixture consisting of 0.15 M EPPS-NaOH (pH 8.0) with 6 mM MgCl₂, 0.2 mM γ³²P-ATP (0.2-0.4 mCi/μmol), 10% glycerol, 0.1% Triton X-100, and poly E₄Y. Poly E₄Y is a synthetic peptide whose phosphorylation is measured in this assay by the addition of the radioactively labeled phosphate from the ATP (Budde et al., 1995). For screening assays, 50 μg/ml poly E₄Y was used, and for Kᵢ determinations variable concentrations (0, 20, 30, 75, and 150 μg/ml) of poly E₄Y were used. When ATP was varied (0, 50, 100 and 250 μM), poly E₄Y was kept constant at 150 μg/ml.

Compounds were identified as especially good inhibitors of Src if they possessed an IC₅₀ of 10 μM or less. However, all of the disclosed compounds have excellent potential, and numerous other commercial candidates will emerge after further experimentation.

TABLE 2
Formula I Compounds

| | | | | | | | | | % Inhibition |
|---|---|---|---|---|---|---|---|---|---|
| | | | | | | IC50 (μM) | | | at 15 ug/mL |
| Compound # | R1 | R2 | R3 | X1 | Y1 | Src | Csk | FGFr | Src (%) |
| 2 | p-(C6H4)CH3 | p-(C6H4)CH3 | F | — | — | NI | NI | NI | ND |
| 4a | p-(C6H4)CH3 | p-(C6H4)CH3 | p-(C6H4)OCH3 | S | — | 3.4 | 10.8 | 5.8 | ND |
| 4b | p-(C6H4)CH3 | p-(C6H4)CH3 | o-(C6H4)OCH3 | S | — | 7 | 15 | 20 | ND |
| 4c | p-(C6H4)CH3 | p-(C6H4)CH3 | m-(C6H4)OCH3 | S | — | 3.5 | 8.3 | 7.5 | ND |
| 4d | p-(C6H4)CH3 | p-(C6H4)CH3 | p-(C6H4)OH | S | — | 5.5 | 23.9 | 23.9 | ND |
| 4e | p-(C6H4)CH3 | p-(C6H4)CH3 | p-(C6H4)Cl | S | — | 8 | 23 | 1.7 | ND |
| 4f | p-(C6H4)CH3 | p-(C6H4)CH3 | o-(C6H4)Cl | S | — | 2.5 | 10.8 | 10.8 | ND |
| 4g | p-(C6H4)CH3 | p-(C6H4)CH3 | m-(C6H4)Cl | S | — | 1.6 | 9.1 | 5.8 | ND |
| 4h | p-(C6H4)CH3 | p-(C6H4)CH3 | p-(C6H4)F | S | — | 1.8 | 8.6 | 7.8 | ND |
| 4i | p-(C6H4)CH3 | p-(C6H4)CH3 | p-(C6H4)CH3 | S | — | 1.7 | 10.3 | 5.1 | ND |
| 4j | p-(C6H4)CH3 | p-(C6H4)CH3 | o-(C6H4)CH3 | S | — | 1.7 | 12 | 8.6 | ND |
| 4k | p-(C6H4)CH3 | p-(C6H4)CH3 | m-(C6H4)CH3 | S | — | 1.9 | 9.4 | 3.1 | ND |
| 4l | p-(C6H4)CH3 | p-(C6H4)CH3 | 3,5-(C6H3)(CH3)2 | S | — | 1.5 | 6.4 | 3.7 | ND |
| 4m | p-(C6H4)CH3 | p-(C6H4)CH3 | 2,6-(C6H3)(CH3)2 | S | — | 1 | 5.4 | 4.7 | ND |
| 4n | p-(C6H4)CH3 | p-(C6H4)CH3 | o-(C6H4)[CH(CH3)2] | S | — | 1.3 | 5.7 | 4.3 | ND |
| 4o | p-(C6H4)CH3 | p-(C6H4)CH3 | C6H5 | S | — | 1.3 | 5.7 | 1.8 | ND |
| 4p | p-(C6H4)CH3 | p-(C6H4)CH3 | 2-furyl | S | CH2 | 1.6 | 11.3 | 7.3 | ND |
| 4q | p-(C6H4)CH3 | p-(C6H4)CH3 | p-(C6H4)Cl | S | CH2 | 1 | 4.1 | 2.7 | ND |
| 4r | p-(C6H4)CH3 | p-(C6H4)CH3 | o-(C6H4)Cl | S | CH2 | 0.65 | 4.2 | 1.9 | ND |
| 4s | p-(C6H4)CH3 | p-(C6H4)CH3 | p-(C6H4)OCH3 | S | CH2 | 1.3 | 5.7 | 4.3 | ND |
| 4t | p-(C6H4)CH3 | p-(C6H4)CH3 | C6H5 | S | CH2 | 1.2 | 5.5 | ND | ND |
| 4u | CH3 | CH3 | p-(C6H4)Cl | S | — | 590 | 333 | 188 | ND |
| 6 | p-(C6H4)CH3 | p-(C6H4)CH3 | Cl | — | — | 1060 | 172 | 20.2 | ND |
| 8 | p-(C6H4)CH3 | p-(C6H4)CH3 | C6H5 | — | — | 1.7 | 10.4 | 4.9 | ND |
| 9a | p-(C6H4)CH3 | p-(C6H4)CH3 | morpholin-4-yl | — | — | ND | ND | ND | 13* |
| 9b | p-(C6H4)CH3 | p-(C6H4)CH3 | N-Me-piperazin-1-yl | — | — | ND | ND | ND | 2 |
| 9c | p-(C6H4)CH3 | p-(C6H4)CH3 | thiomorpholin-4-yl | — | — | ND | ND | ND | 15 |
| 9d | p-(C6H4)CH3 | p-(C6H4)CH3 | 3-pyridyl | NH | CH2 | NI | ND | ND | 19* |
| 9e | p-(C6H4)CH3 | p-(C6H4)CH3 | 2-pyridyl | NH | CH2 | ND | ND | ND | 15 |
| 9f | p-(C6H4)CH3 | p-(C6H4)CH3 | p-(C6H4)OCH3 | NH | CH2 | ND | ND | ND | 17 |
| 9g | p-(C6H4)CH3 | p-(C6H4)CH3 | o-(C6H4)Cl | NH | CH2 | 54 | ND | ND | 65 |
| 9h | p-(C6H4)CH3 | p-(C6H4)CH3 | cyclohexyl | NH | CH2 | ND | ND | ND | 22 |
| 9i | p-(C6H4)CH3 | p-(C6H4)CH3 | cyclohexyl-1-ol | NH | CH2 | ND | ND | ND | 18 |
| 9j | p-(C6H4)CH3 | p-(C6H4)CH3 | cyclohexyl | O | — | ND | ND | ND | 4 |
| 9k | p-(C6H4)CH3 | p-(C6H4)CH3 | 5-Me-pyrazol-4-yl | O | (CH2)3 | ND | ND | ND | 13 |

*= assayed at 50 μg/mL
NI = not determined
NI = no inhibition at 100 μg/mL

TABLE 3
Formula II Compounds

| Compound # | R4 | R5 | R6 |
|---|---|---|---|
| 2 | p-(C6H4)CH3 | p-(C6H4)CH3 | F |
| 6 | p-(C6H4)CH3 | p-(C6H4)CH3 | Cl |
| 12a | p-(C6H4)(CH2CH3) | p-(C6H4)(CH2CH3) | p-(C6H4)OCH3 |
| 12b | p-(C6H4)[CH(CH3)2] | p-(C6H4)[CH(CH3)2] | p-(C6H4)OCH3 |
| 12c | p-(C6H4)Cl | p-(C6H4)Cl | p-(C6H4)OCH3 |
| 12d | p-(C6H4)F | p-(C6H4)F | p-(C6H4)OCH3 |
| 12e | p-(C6H4)OC6H5 | p-(C6H4)OC6H5 | p-(C6H4)OCH3 |
| 12f | m-(C6H4)NO2 | m-(C6H4)NO2 | p-(C6H4)OCH3 |
| 12g | CH2(C6H5) | CH2(C6H5) | p-(C6H4)OCH3 |
| 12h | 1-naphthyl | 1-naphthyl | p-(C6H4)OCH3 |
| 12i | 2-naphthyl | 2-naphthyl | p-(C6H4)OCH3 |
| 12j | p-(C6H4)NH(C═O)CH3 | p- | p-(C6H4)OCH3 |
| | | (C6H4)NH(C═O)CH3 | |
| 12k | p-(C6H4)OCH3 | p-(C6H4)OCH3 | p-(C6H4)OCH3 |
| 12l | (CH2)3CH3 | (CH2)3CH3 | p-(C6H4)OCH3 |
| 12m | 3,4-(C6H3)(OCH3)2 | 3,4-(C6H3)(OCH3)2 | p-(C6H4)OCH3 |
| 12n | C6H5 | C6H5 | p-(C6H4)OCH3 |
| 12o | p-(C6H4)[C(CH3)]3 | p-(C6H4)[C(CH3)]3 | p-(C6H4)OCH3 |
| 12p | p-(C6H4)CH3 | p-(C6H4)CH3 | p-(C6H4)OCH3 |
| 12q | 2,4,6-(C6H2)(CH3)3 | 2,4,6-(C6H2)(CH3)3 | p-(C6H4)OCH3 |
| 12r | p-(C6H4)NO2 | p-(C6H4)NO2 | p-(C6H4)OCH3 |
| 12s | p-(C6H4)CH3 | p-(C6H4)CH3 | (CH2)4CH2OH |
| 12t | p-(C6H4)CH3 | p-(C6H4)CH3 | p-(C6H4)F |
| 12u | p-(C6H4)CH3 | p-(C6H4)CH3 | 2-naphthyl |
| 12v | p-(C6H4)CH3 | p-(C6H4)CH3 | CH3 |
| 12w | p-(C6H4)CH3 | p-(C6H4)CH3 | p-(C6H4)Cl |
| 12x | p-(C6H4)CH3 | p-(C6H4)CH3 | m-(C6H4)CO2H |
| 12y | p-(C6H4)CH3 | p-(C6H4)CH3 | m-(C6H4)CH2CO2H |
| 12z | p-(C6H4)CH3 | p-(C6H4)CH3 | p-(C6H4)CO2H |
| 12aa | p-(C6H4)CH3 | p-(C6H4)CH3 | p-(C6H4)CH2CO2H |
| 12bb | p-(C6H4)CH3 | p-(C6H4)CH3 | CH2CH2CH2OH |
| 12cc | p-(C6H4)CH3 | p-(C6H4)CH3 | allyl |
| 12dd | p-(C6H4)CH3 | p-(C6H4)CH3 | (CH2)7CH2OH |
| 12ee | p-(C6H4)CH3 | p-(C6H4)CH3 | (CH2)7CH2OC(═0)CH3 |
| 12ff | p-(C6H4)CH3 | p-(C6H4)CH3 | p-(C6H4)Cl |
| 12gg | p-(C6H4)CH3 | p-(C6H4)CH3 | p-(C6H4)OCH3 |
| 12hh | p-(C6H4)CH3 | p-(C6H4)CH3 | (CH2)3CH3 |
| 15a | p-(C6H4)CH3 | p-(C6H4)CH3 | p-(C6H4)OCH3 |
| 15b | p-(C6H4)CH3 | p-(C6H4)CH3 | p-(C6H4)Cl |
| 15c | p-(C6H4)CH3 | p-(C6H4)CH3 | m-(C6H4)Cl |
| 15d | p-(C6H4)CH3 | p-(C6H4)CH3 | o-(C6H4)Cl |
| 15e | p-(C6H4)CH3 | p-(C6H4)CH3 | p-(C6H4)OCH3 |
| 15f | p-(C6H4)CH3 | p-(C6H4)CH3 | cyclohexyl |
| 15g | p-(C6H4)CH3 | p-(C6H4)CH3 | o-(C6H4)Cl |
| 17a | p-(C6H4)CH3 | p-(C6H4)CH3 | pyrazol-1-yl |
| 17b | p-(C6H4)CH3 | p-(C6H4)CH3 | benzimidazol-1-yl |
| 17c | p-(C6H4)CH3 | p-(C6H4)CH3 | N(CH3)2 |
| 17d | p-(C6H4)CH3 | p-(C6H4)CH3 | imidazol-1-yl |
| 17e | p-(C6H4)CH3 | p-(C6H4)CH3 | N-(4- |
| | | | toluenesulfonyl)piperazin- |
| | | | 1-yl |
| 17f | p-(C6H4)CH3 | p-(C6H4)CH3 | morpholin-4-yl |
| 21 | p-(C6H4)CH3 | p-(C6H4)CH3 | p-(C6H4)Cl |
| 22a | p-(C6H4)CH3 | p-(C6H4)CH3 | — |
| 22b | p-(C6H4)CH3 | p-(C6H4)CH3 | p-CH2CH2(C6H4)OCH3 |
| 22c | p-(C6H4)CH3 | p-(C6H4)CH3 | C(CH3)3 |
| 22d | p-(C6H4)CH3 | p-(C6H4)CH3 | 3-pyridyl |
| 22e | p-(C6H4)CH3 | p-(C6H4)CH3 | C6H5 |
| 22f | p-(C6H4)CH3 | p-(C6H4)CH3 | CH2C(CH3)2CH2OH |
| 22g | p-(C6H4)CH3 | p-(C6H4)CH3 | CH2(naphth-1-yl) |
| 22h | p-(C64H)CH3 | p-(C6H4)CH3 | CH2C6H5 |
| 22i | p-(C6H4)CH3 | p-(C6H4)CH3 | 2-thienyl |
| 22j | p-(C6H4)CH3 | p-(C6H4)CH3 | 3,5-dimethylisoxazol-4-yl |
| 22k | p-(C6H4)CH3 | p-(C6H4)CH3 | OH |
| 22l | p-(C6H4)CH3 | p-(C6H4)CH3 | OH |
| 22m | p-(C6H4)CH3 | CH3 | p-(C6H4)OCH3 |
| 22n | p-(C6H4)CH3 | 3.5-dimethylisoxazol- | p-(C6H4)OCH3 |
| | | 4-yl | |
| 22o | p-(C6H4)CH3 | 1-methylimidazol-4- | p-(C6H4)OCH3 |
| | | yl | |
| 22p | CH3 | p-(C6H4)CH3 | p-(C6H4)OCH3 |
| 22q | 4-methyl-2- | p-(C6H4)CH3 | p-(C6H4)OCH3 |
| | acetamidothiazol-5-yl | | |
| 22r | 3,5-dimethylisoxazol-4-yl | p-(C6H4)CH3 | p-(C6H4)OCH3 |
| 22s | 1-methylimidazol-4-yl | p-(C6H4)CH3 | p-(C6H4)OCH3 |
| 22t | 5-Br-6-Cl-pyrid-3-yl | p-(C6H4)CH3 | p-(C6H4)OCH3 |
| 22u | 7-Cl- | p-(C6H4)CH3 | p-(C6H4)OCH3 |
| | benzo[1,2,5]oxadiazol-4- | | |
| | yl | | |
| 22v | 5-[3-(isoxazolyl)]thien-2- | p-(C6H4)CH3 | p-(C6H4)OCH3 |
| | yl | | |
| 22w | 1,2,5-trimethyl-3- | p-(C6H4)CH3 | p-(C6H4)OCH3 |
| | carbomethoxypyrrol-4-yl | | |
| 22x | p- | p-(C6H4)CH3 | p-(C6H4)OCH3 |
| | (C6H4)CH2CH2CH2CH3 | | |
| 22y | 2-(1-naphthyl)ethyl | p-(C6H4)CH3 | p-(C6H4)OCH3 |
| 22z | p-(C6H4)SO2CH3 | p-(C6H4)CH3 | p-(C6H4)OCH3 |
| 22aa | m-(C6H4)OCH3 | p-(C6H4)CH3 | p-(C6H4)OCH3 |
| 22bb | 5-bromothien-2-yl | p-(C6H4)CH3 | p-(C6H4)OCH3 |
| 22cc | isoquinolin-5-yl | p-(C6H4)CH3 | p-(C6H4)OCH3 |
| 22dd | p-(C6H4)CH3 | 1,2,5-trimethyl-3- | p-(C6H4)OCH3 |
| | | carbomethoxypyrrol- | |
| | | 4-yl | |
| 22ee | p-(C6H4)CH3 | 2-(1-naphthyl)ethyl | p-(C6H4)OCH3 |
| 22ff | p-(C6H4)CH3 | p-(C6H4)SO2CH3 | p-(C6H4)OCH3 |
| 22gg | p-(C6H4)CH3 | 5-bromothien-2-yl | p-(C6H4)OCH3 |
| 22hh | p-(C6H4)CH3 | 2-methoxy-4- | p-(C6H4)OCH3 |
| | | methylphenyl | |
| 22ii | p-(C6H4)CH3 | p-(C6H4)CH3 | C6H5 |
| 22jj | p-(C6H4)CH3 | p-(C6H4)CH3 | C6H5 |
| 22kk | p-(C6H4)CH3 | p-(C6H4)CH3 | C6H5 |
| 22ll | CH3 | p-(C6H4)CH3 | o-(C6H4)Cl |
| 22mm | p-(C6H4)CH3 | p-(C6H4)CH3 | o-(C6H4)Cl |
| 22nn | p-(C6H4)CH3 | Me | o-(C6H4)Cl |

| | | | | | | | % Inhibition |
|---|---|---|---|---|---|---|---|
| | | | | IC50 (μM) | | | at 15 μg/mL |
| Compound # | R7 | X2 | Y2 | Src | Csk | FGFr | Src (%) |
| 2 | H | — | — | NI | NI | NI | ND |
| 6 | H | — | — | NI | NI | 736 | ND |
| 12a | H | O | — | 23 | NI | 44 | ND |
| 12b | H | O | — | 18.5 | NI | 45 | ND |
| 12c | H | O | — | 8.6 | 95 | 19 | ND |
| 12d | H | O | — | 24 | 73 | 35 | ND |
| 12e | H | O | — | 19 | NI | 46 | ND |
| 12f | H | O | — | 20 | NI | 35 | ND |
| 12g | H | O | — | 35 | 102 | 70 | ND |
| 12h | H | O | — | 9 | 98 | 36 | ND |
| 12i | H | O | — | 8 | NI | 48 | ND |
| 12j | H | O | — | 50 | NI | 192 | ND |
| 12k | H | O | — | 21 | 88 | 44 | ND |
| 12l | H | O | — | 40 | NI | 81 | ND |
| 12m | H | O | — | 54 | NI | NI | ND |
| 12n | H | O | — | 29 | NI | 24 | ND |
| 12o | H | O | — | 7.2 | NI | 22 | ND |
| 12p | H | O | — | 28 | NI | 26 | ND |
| 12q | H | O | — | 39 | NI | 42 | ND |
| 12r | H | O | — | 6.6 | NI | 15 | ND |
| 12s | H | O | — | ND | ND | ND | 9* |
| 12t | H | O | — | 19 | 105 | 40 | ND |
| 12u | H | O | — | 12 | 104 | 38 | ND |
| 12v | H | O | — | 69 | NI | 145 | ND |
| 12w | H | O | — | 5.5 | 185 | 15.3 | ND |
| 12x | H | O | — | 9.5 | 72 | 31 | ND |
| 12y | H | O | — | 11 | 104 | 26 | ND |
| 12z | H | O | — | 9 | 118 | 24 | ND |
| 12aa | H | O | — | 6.7 | 77 | 10.6 | ND |
| 12bb | H | O | — | NI | NI | NI | ND |
| 12cc | H | O | — | ND | ND | ND | 19 |
| 12dd | H | O | — | ND | ND | ND | 36 |
| 12ee | H | O | — | 131 | ND | ND | ND |
| 12ff | H | S | — | 4 | 8.6 | 3.6 | ND |
| 12gg | H | S | — | 16 | NI | 17 | ND |
| 12hh | H | S | — | 18 | NI | 30 | ND |
| 15a | CH2CHCH2 | O | CH2 | 212 | ND | ND | ND |
| 15b | H | O | CH2 | 14 | NI | 88 | ND |
| 15c | H | O | CH2 | 27 | NI | 54 | ND |
| 15d | H | O | CH2 | 14 | 59 | 66 | ND |
| 15e | H | O | CH2 | 15 | NI | 132 | ND |
| 15f | H | O | CH2 | 42 | NI | 91 | ND |
| 15g | H | S | CH2 | 11 | NI | NI | ND |
| 17a | H | — | — | 46 | 214 | 64 | ND |
| 17b | H | — | — | 21 | 83 | ND | ND |
| 17c | H | — | — | 39 | NI | 98 | ND |
| 17d | H | — | — | 51 | NI | 3.1 | ND |
| 17e | H | — | — | 19 | NI | 21 | ND |
| 17f | H | — | — | 38 | NI | 50 | ND |
| 21 | H | — | — | 8.6 | NI | 23 | ND |
| 22a | OCH3 | H | — | NI | NI | NI | ND |
| 22b | H | C(═0) | NH | ND | ND | ND | 25 |
| 22c | H | C(═O) | NH | 300 | ND | ND | ND |
| 22d | H | C(═O) | NH | ND | ND | ND | 0 |
| 22e | H | C(═O) | NH | 149 | ND | ND | ND |
| 22f | H | C(═O) | NH | ND | ND | ND | 3 |
| 22g | H | C(═O) | NH | 18 | ND | ND | ND |
| 22h | H | NH | C(═O) | 47 | ND | ND | ND |
| 22i | H | NH | SO2 | ND | ND | ND | 26 |
| 22j | H | NH | SO2 | 25 | ND | ND | ND |
| 22k | H | C(═O) | — | ND | NI | ND | ND |
| 22l | H | CH2 | — | ND | ND | ND | 5 |
| 22m | H | O | — | ND | ND | ND | 22 |
| 22n | H | O | — | ND | ND | ND | 5 |
| 22o | H | O | — | ND | ND | ND | 0 |
| 22p | H | O | — | ND | ND | ND | 17 |
| 22q | H | O | — | 154 | ND | ND | ND |
| 22r | H | O | — | 335 | ND | ND | ND |
| 22s | H | O | — | ND | ND | ND | 3 |
| 22t | H | O | — | 44 | ND | ND | ND |
| 22u | H | O | — | 20 | ND | ND | ND |
| 22v | H | O | — | ND | ND | ND | 22 |
| 22w | H | O | — | ND | ND | ND | 6 |
| 22x | H | O | — | 36 | ND | ND | ND |
| 22y | H | O | — | 20 | ND | ND | ND |
| 22z | H | O | — | 174 | ND | ND | ND |
| 22aa | H | O | — | 133 | ND | ND | ND |
| 22bb | H | O | — | 19.5 | ND | ND | ND |
| 22cc | H | O | — | 45 | ND | ND | ND |
| 22dd | H | O | — | ND | ND | ND | 5 |
| 22ee | H | O | — | 50 | ND | ND | ND |
| 22ff | H | O | — | ND | ND | ND | 2 |
| 22gg | H | O | — | 24 | ND | ND | ND |
| 22hh | H | O | — | ND | ND | ND | 17 |
| 22ii | H | C(═NOCH2C6H5) | — | 64 | ND | ND | ND |
| 22jj | H | C(═NOH) | — | ND | ND | ND | 16 |
| 22kk | H | C(═NOCH3) | — | ND | ND | ND | 23 |
| 22ll | H | S | CH2 | ND | ND | ND | 17 |
| 22mm | H | S | CH2 | 11 | ND | ND | ND |
| 22nn | H | S | CH2 | ND | ND | ND | 21 |

*= assayed at 50 μg/mL
ND = not determined
NI = no inhibition at 100 μg/mL

TABLE 4
Formula III Compounds

| | | | | | | | | | | % Inhibition |
|---|---|---|---|---|---|---|---|---|---|---|
| | | | | | | | IC50 (μM) | | | at 15 μg/mL |
| Compound # | R8 | R9 | R10 | R11 | X3 | Y3 | Src | Csk | FGFr | Src (%) |
| 23 | p-(C6H4)CH3 | p-(C6H4)CH3 | p-(C6H4)Cl | NH2 | S | — | 58 | 280 | 56 | ND |
| 24 | p-(C6H4)CH3 | p-(C6H4)CH3 | p-(C6H4)Cl | NHC(═O)CH3 | S | — | NI | NI | 515 | ND |
| 33 | p-(C6H4)CH3 | p-(C6H4)CH3 | p-(C6H4)OCH3 | CO2CH3 | O | — | 67 | NI | NI | ND |
| 34 | p-(C6H4)CH3 | p-(C6H4)CH3 | p-(C6H4)OCH3 | CO2H | O | — | 77 | ND | ND | ND |
| 37 | p-(C6H4)CH3 | p-(C6H4)CH3 | p-(C6H4)OCH3 | CH3 | O | — | 118 | ND | ND | ND |
| 38a | p-(C6H4)CH3 | p-(C6H4)CH3 | Br | Br | — | — | 31 | NI | 110 | ND |
| 38b | p-(C6H4)CH3 | p-(C6H4)CH3 | p-(C6H4)OCH3 | CF3 | O | — | ND | ND | ND | 50 |
| 38c | p-(C6H4)CH3 | p-(C6H4)CH3 | o-(C6H4)Cl | F | S | CH2 | 2.2 | ND | ND | ND |
| 38d | p-(C6H4)CH3 | p-(C6H4)CH3 | o-(C6H4)Cl | F | SO2 | CH2 | 13 | ND | ND | ND |

ND = not determined
NI = no inhibition at 100 μg/mL

TABLE 5
Formula IV Compounds

| Compound | R12 | R13 | R14 | R15 | A |
|---|---|---|---|---|---|
| 42 | p-(C6H4)CH3 | OP(═O)(OCH2CH3)2 | p-(C6H4)OCH3 | H | NHSO2 |
| 43 | p-(C6H4)CH3 | OP(═O)(OH)2 | p-(C6H4)OCH3 | H | NHSO2 |
| 48 | p-(C6H4)CH3 | p-(C6H4)CH3 | p-(C6H4)OCH3 | H | O-SO2 |
| 51 | p-(C6H4)CH3 | p-(C6H4)CH3 | p-(C6H4)Cl | NO2 | NHC(═O) |
| 52 | p-(C6H4)CH3 | p-(C6H4)CH3 | p-(C6H4)Cl | H | NHC(═O) |
| 53 | p-(C6H4)CH3 | p-(C6H4)CH3 | p-(C6H4)Cl | NO2 | N(CH3)SO2 |
| 55 | p-(C6H4)CH3 | NH2 | p-(C6H4)Cl | NO2 | NHSO2 |
| 56 | p-(C6H4)CH3 | NH2 | p-(C6H4)Cl | NO2 | NHC(═O) |
| 59 | p-(C6H4)CH3 | OH | p-(C6H4)OCH3 | H | NHSO2 |
| 60 | p-(C6H4)CH3 | OCH3 | p-(C6H4)OCH3 | H | NHSO2 |
| 61 | p-(C6H4)CH3 | OCH3 | p-(C6H4)OCH3 | H | N(CH3)SO2 |
| 62 | p-(C6H4)CH3 | CH2CH2OH | p-(C6H4)OCH3 | H | NHSO2 |
| 63 | p-(C6H4)CH3 | CH2CH2CH2OH | p-(C6H4)OCH3 | H | NHSO2 |
| 64 | p-(C6H4)CH3 | rac-CH2CH(OH)CH2OH | p-(C6H4)OCH3 | H | NHSO2 |
| 65 | p-(C6H4)CH3 | CH2CO2H | p-(C6H4)OCH3 | H | NHSO2 |
| 66 | OCH3 | p-(C6H4)CH3 | p-(C6H4)OCH3 | H | — |
| 67 | p-(C6H4)CH3 | CO2CH3 | p-(C6H4)OCH3 | H | NHSO2 |
| 68 | p-(C6H4)CH3 | CO2H | p-(C6H4)OCH3 | H | NHSO2 |
| 69 | p-(C6H4)CH3 | H | p-(C6H4)Cl | NO2 | NHSO2 |
| 70 | H | p-(C6H4)CH3 | p-(C6H4)Cl | NO2 | — |
| 71a | NH2 | p-(C6H4)CH3 | p-(C6H4)OCH3 | H | — |
| 71b | 2-cyanopyrid-5-yl | p-(C6H4)CH3 | p-(C6H4)OCH3 | H | NHC(═O) |
| 71c | 2-trifluoromethylpyrid-5-yl | p-(C6H4)CH3 | p-(C6H4)OCH3 | H | NHC(═O) |
| 71d | p-(C6H4)CN | p-(C6H4)CH3 | p-(C6H4)OCH3 | H | NHCH2 |
| 71e | p-(C6H4)NHC(═O)CH3 | p-(C6H4)CH3 | p-(C6H4)OCH3 | H | NHCH2 |
| 71f | benzofuran-2-yl | p-(C6H4)CH3 | p-(C6H4)OCH3 | H | NHCH2 |
| 71g | quinolin-2-yl | p-(C6H4)CH3 | p-(C6H4)OCH3 | H | NHCH2 |
| 71h | 5-methyl-pyrazin-2-yl | p-(C6H4)CH3 | p-(C6H4)OCH3 | H | NHC(═O) |
| 71i | p-(C6H4)CH3 | NH2 | p-(C6H4)OCH3 | H | CH2NHSO2 |
| 71j | CN | p-(C6H4)CH3 | p-(C6H4)OCH3 | H | — |
| 71k | CO2H | p-(C6H4)CH3 | p-(C6H4)OCH3 | H | — |
| 71l | p-(C6H4)CH3 | NH2 | p-(C6H4)OCH3 | H | NHSO2 |
| 71m | p-(C6H4)CH3 | p-(C6H4)CH3 | p-(C6H4)OCH3 | H | NHSO2 |
| 71n | p-(C6H4)CH3 | CH2NH2 | p-(C6H4)OCH3 | H | NHSO2 |
| 71o | p-(C6H4)CH3 | p-(C6H4)CH3 | C6H5 | H | NHSO2 |
| 71p | p-(C6H4)CH3 | p-(C6H4)CH3 | p-(C6H4)OCH3 | H | NHSO2 |
| 71q | p-(C6H4)CH3 | p-(C6H4)CH3 | p-(C6H4)OCH3 | H | NHSO2 |
| 71r | p-(C6H4)CH3 | p-(C6H4)CH3 | p-(C6H4)OCH3 | H | NHC(═O) |
| 71s | C6H5 | p-(C6H4)CH3 | p-(C6H4)OCH3 | H | NHC(═O)CH2 |
| 71t | NH2 | p-(C6H4)CH3 | p-(C6H4)OCH3 | H | — |
| 71u | p-(C6H4)CH3 | quinolin-2-yl | p-(C6H4)OCH3 | H | NHSO2 |
| 71v | C(═O)NH2 | p-(C6H4)CH3 | p-(C6H4)OCH3 | H | — |
| 71w | CH2NH2 | p-(C6H4)CH3 | p-(C6H4)OCH3 | H | — |
| 71x | CH3 | 6-methylnaphth-2-yl | p-(C6H4)OCH3 | H | CH2NHSO2 |
| 71y | p-(C6H4)CH3 | p-(C6H4)CH3 | morpholin-4-yl | NO2 | NHC(═O) |
| 71z | p-(C6H4)CH3 | CH2CH2CH3 | p-(C6H4)OCH3 | H | NHSO2 |
| 71aa | p-(C6H4)CH3 | p-(C6H4)CH3 | cyclohexyl | NO2 | NHC(═O) |
| 71bb | p-(C6H4)CH3 | p-(C6H4)CH3 | naphth-1-yl | NO2 | NHC(═O) |
| 71cc | p-(C6H4)CH3 | NH2 | p-(C6H4)OCH3 | NO2 | NHSO2 |
| 71dd | p-(C6H4)CH3 | p-(C6H4)CH3 | p-(C6H4)OCH3 | H | NHSO2 |
| 71ee | p-(C6H4)CH3 | NH2 | p-(C6H4)OCH3 | CF3 | NHSO2 |
| 71ff | p-(C6H4)CH3 | p-(C6H4)CH3 | p-(C6H4)OCH3 | H | CH2NHC(═O) |
| 71gg | NH2 | p-(C6H4)CH3 | p-(C6H4)OCH3 | CF3 | — |
| 71hh | p-(C6H4)CH3 | p-(C6H4)CH3 | p-(C6H4)OCH3 | H | NHSO2 |
| 71ii | p-(C6H4)CH3 | p-(C6H4)CH3 | p-(C6H4)OCH3 | H | NHSO2 |
| 71jj | p-(C6H4)CH3 | p-(C6H4)CH3 | p-(C6H4)OCH3 | H | NHCH2 |
| 71kk | NH2 | p-(C6H4)CH3 | o-(C6H4)Cl | H | — |
| 71ll | 7-chloro- | NH2 | o-(C6H4)Cl | NO2 | NHSO2 |
| | benzo[1,2,5]oxadiazol-4-yl | | | | |
| 71mm | p-(C6H4)CH2CH3 | p-(C6H4)CH3 | CH3 | H | SO2NH |
| 71nn | p-(C6H4)CH3 | p-(C6H4)CH3 | o-(C6H4)Cl | H | NHSO2 |

| | | | | | | | % Inhibition |
|---|---|---|---|---|---|---|---|
| | | | | IC50 (μM) | | | at 15 μg/mL |
| Compound | B | X4 | Y4 | Src | Csk | FGFr | Src % |
| 42 | — | O | — | 65 | NI | 109 | ND |
| 43 | — | O | — | 13 | NI | 29 | ND |
| 48 | O-SO2 | CH2 | O | 102 | NI | NI | ND |
| 51 | NHC(═O) | S | — | 153 | 51 | 40 | ND |
| 52 | NHC(═O) | S | — | 51 | NI | NI | ND |
| 53 | N(CH3)SO2 | S | — | 388 | 261 | 254 | ND |
| 55 | — | S | — | 33 | NI | 2.2 | ND |
| 56 | — | S | — | NI | NI | 410 | ND |
| 59 | — | O | — | 65 | NI | 65 | ND |
| 60 | — | O | — | 129 | NI | NI | ND |
| 61 | — | O | — | 177 | NI | NI | ND |
| 62 | O | O | — | 126 | 15 | NI | ND |
| 63 | O | O | — | 212 | NI | NI | ND |
| 64 | O | O | — | 189 | NI | NI | ND |
| 65 | O | O | — | 169 | NI | NI | ND |
| 66 | NHSO2 | CH2 | O | 119 | NI | 162 | ND |
| 67 | — | O | — | NI | NI | 246 | ND |
| 68 | — | O | — | 133 | 303 | NI | ND |
| 69 | — | S | — | 1970 | 986 | NI | ND |
| 70 | NHSO2 | S | — | 599 | 369 | 251 | ND |
| 71a | NHSO2 | O | — | ND | ND | ND | 13 |
| 71b | NHSO2 | O | — | ND | ND | ND | 0 |
| 71c | NHSO2 | O | — | ND | ND | ND | 5 |
| 71d | NHSO2 | O | — | 557 | ND | ND | ND |
| 71e | NHSO2 | O | — | 203 | ND | ND | ND |
| 71f | NHSO2 | O | — | 627 | ND | ND | ND |
| 71g | NHSO2 | O | — | ND | ND | ND | 10 |
| 71h | NHSO2 | O | — | ND | ND | ND | 0 |
| 71i | — | O | — | ND | ND | ND | 13 |
| 71j | NHSO2 | O | — | 254 | ND | ND | ND |
| 71k | NHSO2 | O | — | 262 | ND | ND | ND |
| 71l | — | O | — | 62 | ND | ND | ND |
| 71m | CH2NHSO2 | O | — | 136 | ND | ND | ND |
| 71n | — | O | — | 1300 | ND | ND | ND |
| 71o | N[C(═O)C6H5)] | O | C(═O) | 39 | ND | ND | ND |
| | SO2 | | | | | | |
| 71p | CH2NHC(═O) | O | — | 108 | ND | ND | ND |
| 71q | NHC(═O) | O | — | 99 | ND | ND | ND |
| 71r | NHSO2 | O | — | 159 | ND | ND | ND |
| 71s | NHSO2 | O | — | ND | ND | ND | 10 |
| 71t | N(CH3)SO2 | O | — | ND | ND | ND | 0 |
| 71u | E/Z N═C | O | — | ND | ND | ND | 6 |
| 71v | NHSO2 | CH2 | O | ND | ND | ND | 10 |
| 71w | NHSO2 | CH2 | O | ND | ND | ND | 9 |
| 71x | NHSO2 | CH2 | O | ND | ND | ND | 20 |
| 71y | NHC(═O) | — | — | ND | ND | ND | 14 |
| 71z | NHC(═O) | O | — | ND | ND | ND | 5 |
| 71aa | NHC(═O) | NH | CH2 | ND | ND | ND | 0 |
| 71bb | NHC(═O) | NH | CH2 | ND | ND | ND | 22 |
| 71cc | — | O | — | ND | ND | ND | 15 |
| 71dd | N(CH3)SO2 | O | — | 172 | ND | ND | ND |
| 71ee | — | O | — | ND | ND | ND | 21 |
| 71ff | NHSO2 | CH2 | O | ND | ND | ND | 4 |
| 71gg | NHSO2 | O | — | ND | ND | ND | 19 |
| 71hh | NH2CH2 | O | — | ND | ND | ND | 23 |
| 71ii | NHC(═O) | O | — | ND | ND | ND | 21 |
| | NHSO2 | | | | | | |
| 71jj | NHSO2 | O | — | ND | ND | ND | 19 |
| 71kk | NHSO2 | S | CH2 | ND | ND | ND | 29 |
| 71ll | — | S | CH2 | ND | ND | ND | 26 |
| 71mm | NHSO2 | O | — | ND | ND | ND | 19 |
| 71nn | NHSO2 | SO2 | CH2 | ND | ND | ND | 46 |

ND = not determined
NI = no inhibition at 100 μg/mL

TABLE 6
Formula V Compounds

| | | | | | | | | | % Inhibition |
|---|---|---|---|---|---|---|---|---|---|
| | | | | | | IC50 (μM) | | | at 15 μg/mL |
| Compound# | R16 | R17 | R18 | X5 | Y5 | Src | Csk | FGFr | Src (%) |
| 72a | H | p-(C6H4)CH3 | p-(C6H4)OCH3 | O | C(═O) | ND | ND | ND | 21 |
| 72b | p-(C6H4)CH3 | H | p-(C6H4)OCH3 | O | C(═O) | ND | ND | ND | 19 |
| 72c | p-(C6H4)CH3 | p-(C6H4)CH3 | p-(C6H4)OCH3 | O | C(═O) | ND | ND | ND | 22 |
| 72d | H | H | p-(C6H4)OCH3 | O | SO2 | ND | ND | ND | 12 |
| 72e | H | H | p-(C6H4)OCH3 | O | C(═O) | ND | ND | ND | 4 |
| 72f | p-(C6H4)CH3 | H | p-(C6H4)OCH3 | O | SO2 | ND | ND | ND | 14 |

ND = not determined

### REFERENCES

Atwell, et al, J. Med. Chem. 1972, 15 (611-615).
Audeenko, S A. Russian J. Org. Chem. 1998, 34 (515-524).
Barnekow A: Functional Aspects of the c-src Gene: Crit. Rev. Oncogenesis 1:277-292, 1989.
Bezverkhii, N P, Zinukov, V D, Kremlev, M M. J. Org. Chem USSR (Eng. Trans), 1982, 18 (1959-1960).
Bezverkhii, N P, Zinukov, V D, Kremlev, M M. J. Org. Chem USSR (Eng. Trans), 1984, 20 (303-06, 339-43 and 947-50)
Bjelfman C, Hedborg R, Johansson I, Nordenskjold M, Pahlman S: Expression of the Neuronal form of pp60c-src in Neuroblastoma in Relation to Clinical Stage and Prognosis. Cancer Res 50:6908-6914, 1990.
Budde R J A, Ke S, and Levin V A: Activity of pp60c-src in 60 Different Cell Lines Derived from Human Tumors. Cancer Biochem. Biophys. 14:171-175, 1994.
Budde R J A, Ramdas L, and Ke S: Recombinant Src from Baculovirus-infected Insect Cells: Purification and Characterization. Preparative Biochemistry 23:493-515, 1993.
Cambridge Dictionary of Biology, New York, 1990.
Cartwright C A, Meisler A I, Eckhart W: Activation of the pp60c-src Protein Kinase is an Early Event in Colonic Carcinogenesis. Proc Natl Acad Sci USA. 87:558-562, 1990.
Fanning P, Bulovas K, Saini K S, Libertino J A, Joyce A D, Summerhayes I C: Elevated Expression of pp60c-src in Low Grade Human Bladder Carcinoma. Cancer Res 52:1457-1462, 1992.
Heterocycles, 1987, 26 (2433)
Jessup J M, Gallick G E: The Biology of Colorectal Carcinoma. Curr Problems in Cancer 16:263-328, 1993.
J. Med. Chem. 1963, 6, (599-601).
J. Med. Chem. 1992, 35 (4455).
Kitanaka A, Waki M, Kamono H, Tanaka T: Antisense Src Expression Inhibits Proliferation and Erythropoietin-induced Erythroid Differentiation of K562 Human Leukemia Cells. Biochem Biophysic Res Commun 201:1534-1540, 1994.
Kostic, S., Soskic, V., Joksimovic, J. Arzneim. Forsch. 1994, 44 (697-702).
Lynch S A, Brugge J S, Fromowitz F, Glantz L, Wang P, Caruso R, Viola M V: Increased Expression of the Src Proto-oncogene in the Leukemia and a Subgroup of B-cell Lymphomas. Leukemia 7:1416-1422, 1993.
Merck Manual, The Seventeenth Edition, West Point, Pa., 1999.
Park S H, Kang S H, Lim S H, Oh H S, Lee K H: Design and Synthesis of Small Chemical Inhibitors Containing Different Scaffolds for Lck SH2 Domain. Bioorg & Med Chem Lett 13:3455-3459, 2003.
Partanen S: Immunohistochemically Demonstrated pp60c-src in Human Breast Carcinoma. Oncology Reports 1:603-606, 1994.
Sinha S and Corey S J: Implications for Src Kinases in Hematopoiesis: Signal Transduction Therapeutics. Journal of Hematotherapy & Stem Cell Research 8:465-480, 1999.
Soriano P, Montogomery C, Geske R, Bradley A: Targeted Disruption of the c-src Proto-oncogene Leads to oOteopetrosis in Mice. Cell 64:693-702, 1991.
Sridhar R, Hanson-Painton O, Cooper D R: Protein Kinases as Therapeutic Targets. Pharm Res 17:1345-1353, 2000.
Staley C, Parikh N, and Gallick G: Decreased Tumorigenicity of a Human Colon Adenocarcinoma Cell Line by an Antisense Expression Vector Specific for Src. Cell Growth & Differentiation 8:269-274, 1997.
Takeshima E, Hamaguchi M, Watanabe T, Akiyama S, Kataoka M, Ohnishi Y, Xiao H, Nagai Y, Takaagi H: Aberrant Elevation of Tyrosine-specific Phosphorylation in Human Gastric Cancer Cells. Japan J Cancer Res 82:1428-1435, 1991.
Termuhlen P M, Curley S A, Talamonti M S, Saboorian M H, Gallick G E: Site-specific Differences in pp60c-src Activity in Human Colorectal Metastases. J Surg Res 54:293-298, 1993.
U.S. Pat. No. 2,258,162 (1938).
U.S. Pat. No. 6,503,914 (2003).
Waki M, Kitanaka A, Kamano H, Tanaka T, Kubota Y, Ohnishi H, Takahara J, Irino S: Antisense SRC Expression Inhibits U937 Human Leukemia Cell Proliferation in Conjunction with Reduction of c-MYB Expression. Biochem Biophys Res Commun 201:1001-1007, 1994.
Wiener J R, Nakano K, Kruzelock R P, Bucana C D, Bast R C, Gallick, G E: Decreased Src Tyrosine Kinase Activity Inhibits Malignant Human Ovarian Cancer Tumor Growth in a Nude Mouse Model. Clinical Cancer Research 5: 2164-2170, 1999.

What is claimed:

1. A compound having a structure selected from the group consisting of Formulas I through V:
selected from the following general formulas: 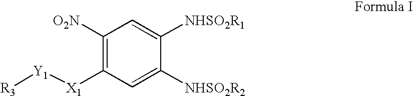
wherein R₁ is p-(C₆H₄)CH₃ or CH₃;
wherein R₂ is p-(C₆H₄)CH₃ or CH₃;
wherein R₃ is F, Cl, p-(C₆H₄)OCH₃, o-(C₆H₄)OCH₃, m-(C₆H₄)OCH₃, p-(C₆H₄)OH, p-(C₆H₄)Cl, o-(C₆H₄)Cl, m-(C₆H₄)Cl, p-(C₆H₄)F, p-(C₆H₄)CH₃, o-(C₆H₄)CH₃, m-(C₆H₄)CH₃, 3,5-(C₆H₃)(CH₃)₂, 2,6-(C₆H₃)(CH₃)₂, o-(C₆H₄)[CH(CH₃)₂], C₆H₅, 2-furyl, morpholin-4-yl, n-Me-piperazin-1-yl, thiomorpholin-4-yl, 3-pyridyl, 2-pyridyl, cyclohexyl, cyclohexyl-1-ol, or 5-Me-pyrazol-4-yl;
wherein X₁ is S, NH or O; and
wherein Y₁ is (CH₂)ₙ wherein n ranges from 1 to 3; 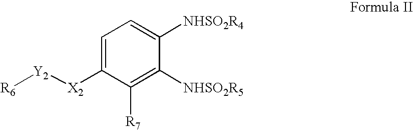
wherein R₄ is p-(C₆H₄)CH₃, p-(C₆H₄)(CH₂CH₃), p-(C₆H₄)[CH(CH₃)₂], p-(C₆H₄)Cl, p-(C₄H₄)F, p-(C₆H₄)OC₆H₅, m-(C₆H₄)NO₂, CH₂(C₆H₅), 1-naphthyl, 2-naphthyl, p-(C₆H₄)NH(C═O)CH3, p-(C₆H₄)OCH₃, (CH₂)₃CH₃, 3,4-(C₆H₃)(OCH₃)₂, C₆H₅, p-(C₆H₄)[C(CH₃)]₃, 2,4,6-(C₆H₂)(CH₃)₃, p-(C₆H₄)NO₂, CH₃, 4-methyl-2-acetamidothiazol-5-yl, 3,5-dimethylisoxazol-4-yl, 1-methylimidazol-4-yl, 5-Br-6-Cl-pyrid-3-yl, 7-Cl-benzo[1,2,5]oxadiazol-4-yl, 5-[3-(isoxazolyl)]thien-2-yl, 1,2,5-trimethyl-3-carbomethoxypyrrol-4-yl, p-(C₆R₄)CH₂CH₂CH₂CH₃, 2-(1-naphthyl)ethyl, p-(C₆H₄)SO₂CH₃, m-(C₆H₄)OCH₃, 5-bromothien-2-yl, or isoquinolin-5-yl;
wherein R₅ is p-(C₆H₄)CH₃, p-(C₆H₄)(CH₂CH₃), p-(C₆H₄)[CH(CH₃)₂], p-(C₆H₄)Cl, p-(C₆H₄)F, p-(C₆H₄)OC₆H₅, m-(C₆H₄)NO₂, CH₂(C₆H₅), 1-naphthyl, 2-naphthyl, p-(C₆H₄)NH(C═O)CH₃, p-(C₆H₄)OCH₃, (CH₂)₃CH₃, 3,4-(C₆H₃)(OCH₃)₂, C₆H₅, p-(C₆H₄)[C(CH₃)]₃, 2,4,6-(C₆H₂)(CH₃)₃, p-(C₆H₄)NO₂, CH₃, 3,5-dimethylisoxazol-4-yl, 1-methylimidazol-4-yl, 1,2,5-trimethyl-3-carbomethoxypyrrol-4-yl, 2-(1-naphthyl)ethyl, p-(C₆H₄)SO₂CH₃, 5-bromothien-2-yl, or 2-methoxy-4-methylphenyl;
wherein R₆ is F, Cl, p-(C₆H₄)OCH₃, (CH₂)₄CH₂OH, p-(C₆H₄)F, 2-naphthyl, CH₃, p-(C₆H₄)Cl, m-(C₆H₄)CO₂H, m-(C₆H₄)CH₂CO₂H, p-(C₆H₄)CO₂H, p-(C₆H₄)CH₂CO₂H, CH₂CH₂CH₂OH, allyl, (CH₂)₇CH₂OH, (CH₂)₇CH₂C(═O)CH₃, (CH₂)₃CH₃, m-(C₆H₄)Cl, o-(C₆H₄)Cl, cyclohexyl, pyrazol-1-yl, benzimidazol-1-yl, N(CH₃)₂, imidazol-1-yl, N-(4-toluenesulfonyl)piperazin-1-yl, morpholin-4-yl, p-CH₂CH₂(C₆H₄)OCH₃, C(CH₃)₃, 3-pyridyl, C₆H₅, CH₂C(CH₃)₂CH₂OH, CH₂(naphth-1-yl), CH₂C₆H₅, 2-thienyl, 3,5-dimethylisoxazol-4-yl, or OH;
wherein R₇ is H, CH₂CHCH₂ or OCH₃;
wherein X₂ is O, S, H, C(═O), NH, CH₂, C(═NOCH₂C₆H₅), C(═NOH), or C(═NOCH₃); and
wherein Y₂ is CH₂, NH, C(═O), or SO₂; 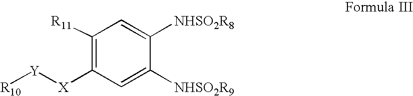
wherein R₈ is p-(C₆H₄)CH₃;
wherein R₉ is p-(C₆H₄)CH₃;
wherein R₁₀ is p-(C₆H₄)Cl, p-(C₆H₄)OCH₃, Br, or o-(C₆H₄)Cl,
wherein R₁₁ is NH₂, NHC(═O)CH₃, CO₂CH₃, CO₂H, CH₃, Br, CF₃, or F;
wherein X₃ is S, O, or SO₂; and
wherein Y₃ is CH₂; 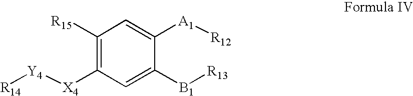
wherein R₁₂ is p-(C₆H₄)CH₃, OCH₃, H, NH₂, 2-cyanopyrid-5-yl, 2-trifluoromethylpyrid-5-yl, p-(C₆H₄)CN, p-(C₆H₄)NHC(═O)CH₃, benzofuran-2-yl, quinolin-2-yl, 5-methyl-pyrazin-2-yl, CN, CO₂H, C₆H₅, C(═O)NH₂, CH₂NH₂, CH₃, 7-chloro-benzo[1,2,5]oxadiazol-4-yl, and p-(C₆H₄)CH₂CH₃,
wherein R₁₃ is p-(C₆H₄)CH₃, OP(═O)(OCH₂CH₃)₂, OP(═O)(OH)₂, p-(C₆H₄)CH₃, NH₂, OH, OCH₃, CH₂CH₂OH, CH₂CH₂CH₂OH, rac-CH₂CH(OH)CH₂OH, CH₂CO₂H, CO₂CH₃, CO₂H, H, CH₂NH₂, quinolin-2-yl, 6-methylnaphth-2-yl, or CH₂CH₂CH₃;
wherein R₁₄ is p-(C₆H₄)OCH₃, p-(C₆H₄)C₁, C₆H₅, morpholin-4-yl, cyclohexyl, naphth-1-yl, o-(C₆H₄)Cl, or CH₃;
wherein R₁₅ is H, NO₂, or CF₃;
wherein X₄ O, CH₂, S, NH, or SO₂;
wherein Y₄ is O, —C(═O), or CH₂;
wherein A is NHSO₂, O—SO₂, NHC(═O), N(CH₃)SO₂, NHCH₂, CH₂NHSO₂, NHC(═O)CH₂, CH₂NHC(═O), or SO₂NH;
wherein B is O—SO₂, NHC(═O), N(CH₃)SO₂, O, NHSO₂, CH₂NHSO₂, N[C(═O)C₆H₅)]SO₂, CH₂NHC(═O), E/Z N═C, NHC(═O), or NH₂CH₂, NHC(═O)NHSO₂; and 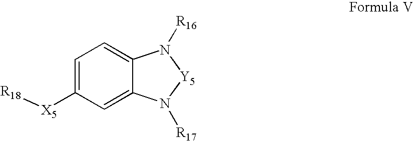
wherein R₁₆ is p-(C₆H₄)CH₃ or H;
wherein R₁₇ is p-(C₆H₄)CH₃ or H;
wherein R₁₈ is p-(C₆H₄)OCH₃;
wherein X₅ is O; and
wherein Y₅ is C(═O) or SO₂.

2. The compound of claim 1 wherein the compound is selected from the group consisting of:
In an embodiment, the PTK inhibitor compound is selected from the group consisting of:
N-[4-fluoro-5-nitro-2-(4-toluenesulfonylamino)-phenyl]-4-toluenesulfonamide;
N-[4-(4-methoxy-phenylsulfanyl)-5-nitro-2-(4-toluenesulfonylamino)-phenyl]-4-toluenesulfonamide;
N-[4-(2-methoxy-phenylsulfanyl)-5-nitro-2-(4-toluenesulfonylamino)-phenyl]-4-toluenesulfonamide;
N-[4-(3-methoxy-phenylsulfanyl)-5-nitro-2-(4-toluenesulfonylamino)-phenyl]-4-toluenesulfonamide;
N-[4-(4-hydroxy-phenylsulfanyl)-5-nitro-2-(4-toluenesulfonylamino)-phenyl]-4-toluenesulfonamide;
N-[4-(4-chloro-phenylsulfanyl)-5-nitro-2-(4-toluenesulfonylamino)-phenyl]-4-toluenesulfonamide;
N-[4-(2-chloro-phenylsulfanyl)-5-nitro-2-(4-toluenesulfonylamino)-phenyl]-4-toluenesulfonamide;
N-[4-(3-chloro-phenylsulfanyl)-5-nitro-2-(4-toluenesulfonylamino)-phenyl]-4-toluenesulfonamide;
N-[4-(4-fluoro-phenylsulfanyl)-5-nitro-2-(4-toluenesulfonylamino)-phenyl]-4-toluenesulfonamide;
N-[5-nitro-2-(4-toluenesulfonylamino)-4-(p-tolylsulfanyl)-phenyl]-4-toluenesulfonamide;
N-[5-nitro-2-(4-toluenesulfonylamino)-4-(o-tolylsulfanyl)-phenyl]-4-toluenesulfonamide;
N-[5-nitro-2-(4-toluenesulfonylamino)-4-(m-tolylsulfanyl)-phenyl]-4-toluenesulfonamide;
N-[4-(2,4-dimethyl-phenylsulfanyl)-5-nitro-2-(4-toluenesulfonylamino)-phenyl]-4-toluenesulfonamide;
N-[4-(2,6-dimethyl-phenylsulfanyl)-5-nitro-2-(4-toluenesulfonylamino)-phenyl]-4-toluenesulfonamide;
N-[4-(2-isopropyl-phenylsulfanyl)-5-nitro-2-(4-toluenesulfonylamino)-phenyl]-4-toluenesulfonamide;
N-[5-nitro-4-phenylsulfanyl-2-(4-toluenesulfonylamino)-phenyl]-4-toluenesulfonamide;
N-[4-(furan-2-ylmethylsulfanyl)-5-nitro-2-(4-toluenesulfonylamino)-phenyl]-4-toluenesulfonamide;
N-[4-(4-chloro-benzylsulfanyl)-5-nitro-2-(4-toluenesulfonylamino)-phenyl]-4-toluenesulfonamide;
N-[4-(2-chloro-benzylsulfanyl)-5-nitro-2-(4-toluenesulfonylamino)-phenyl]-4-toluenesulfonamide;
N-[4-(4-methoxy-benzylsulfanyl)-5-nitro-2-(4-toluenesulfonylamino)-phenyl]-4-toluenesulfonamide;
N-[4-benzylsulfanyl-5-nitro-2-(4-toluenesulfonylamino)-phenyl]-4-toluenesulfonamide;
N-[5-(4-chloro-phenylsulfanyl)-2-methanesulfonylamino-4-nitro-phenyl]-methanesulfonamide;
N-[4-chloro-5-nitro-2-(4-toluenesulfonylamino)-phenyl]-4-toluenesulfonamide;
N-[5-nitro-4-phenyl-2-(4-toluenesulfonylamino)-phenyl]-4-toluenesulfonamide;
N-[4-(morpholin-4-yl)-5-nitro-2-(4-toluenesulfonylamino)-phenyl]-4-toluenesulfonamide;
N-[4-(4-methyl-piperazin-1-yl)-5-nitro-2-(4-toluenesulfonylamino)-phenyl]-4-toluenesulfonamide;
N-[5-nitro-4-(thiomorpholin-4-yl)-2-(4-toluenesulfonylamino)-phenyl]-4-toluenesulfonamide;
N-[5-nitro-4-[(pyridin-3-ylmethyl)-amino]-2-(4-toluenesulfonylamino)-phenyl]-4-toluenesulfonamide;
N-[5-nitro-4-[(pyridin-2-ylmethyl)-amino]-2-(4-toluenesulfonylamino)-phenyl]-4-toluenesulfonamide;
N-[4-(4-methoxy-benzylamino)-5-nitro-2-(4-toluenesulfonylamino)-phenyl]-4-toluenesulfonamide;
N-[4-(2-chloro-benzylamino)-5-nitro-2-(4-toluenesulfonylamino)-phenyl]-4-toluenesulfonamide;
N-[4-(cyclohexylmethyl-amino)-5-nitro-2-(4-toluenesulfonylamino)-phenyl]-4-toluenesulfonamide;
N-[4-(1-hydroxy-cyclohexylmethyl-amino)-5-nitro-2-(4-toluenesulfonylamino)-phenyl]-4-toluenesulfonamide;
N-[4-cyclohexylamino-5-nitro-2-(4-toluenesulfonylamino)-phenyl]-4-toluenesulfonamide;
N-[4-[3-(5-methyl-1H-pyrazol-4-yl)-propylamino]-5-nitro-2-(4-toluenesulfonylamino)-phenyl]-4-toluenesulfonamide;
N-[4-fluoro-2-(4-toluenesulfonylamino)-phenyl]-4-toluenesulfonamide;
N-[4-chloro-2-(4-toluenesulfonylamino)-phenyl]-4-toluenesulfonamide;
4-ethyl-N-[2-(4-ethyl-benzenesulfonylamino)-4-(4-methoxy-phenoxy)-phenyl]-benzenesulfonamide;
4-isopropyl-N-[2-(4-isopropyl-benzenesulfonylamino)-4-(4-methoxy-phenoxy)-phenyl]-benzenesulfonamide;
4-chloro-N-[2-(4-chloro-benzenesulfonylamino)-4-(4-methoxy-phenoxy)-phenyl]-benzenesulfonamide;
4-fluoro-N-[2-(4-fluoro-benzenesulfonylamino)-4-(4-methoxy-phenoxy)-phenyl]-benzenesulfonamide;
N-[4-(4-methoxy-phenoxy)-2-(4-phenoxy-benzenesulfonylamino)-phenyl]-4-phenoxy-benzenesulfonamide;
N-[4-(4-methoxy-phenoxy)-2-(3-nitro-benzenesulfonylamino)-phenyl]-3-nitro-benzenesulfonamide;
N-[4-(4-methoxy-phenoxy)-2-phenylmethanesulfonylamino-phenyl]-C-phenyl-methanesulfonamide;
naphthalene-1-sulfonic acid [4-(4-methoxy-phenoxy)-2-(naphthalen-1-yl-sulfonylamino)-phenyl]-amide;
naphthalene-2-sulfonic acid [4-(4-methoxy-phenoxy)-2-(naphthalen-2-yl-sulfonylamino)-phenyl]-amide;
N-[2-(4-acetamido-benzenesulfonylamino)-4-(4-methoxy-phenoxy)-phenyl]-4-acetamido-benzenesulfonamide;
N-[2-(4-methoxy-benzenesulfonylamino)-4-(4-methoxy-phenoxy)-phenyl]-4-methoxy-benzenesulfonamide;
butane-1-sulfonic acid [2-(butane-1-sulfonylamino)-4-(4-methoxy-phenoxy)-phenyl]-amide;
N-[2-(3,4-dimethoxy-benzenesulfonylamino)-4-(4-methoxy-phenoxy)-phenyl]-3,4-dimethoxy-benzenesulfonamide;
N-[2-benzenesulfonylamino-4-(4-methoxy-phenoxy)-phenyl]-benzenesulfonamide;
N-[2-(4-t-butyl-benzenesulfonylamino)-4-(4-methoxy-phenoxy)-phenyl]-4-t-butyl-benzenesulfonamide;
N-[4-(4-methoxy-phenoxy)-2-(4-toluenesulfonylamino)-phenyl]-4-toluenesulfonamide;
N-[4-(4-methoxy-phenoxy)-2-(2,4,6-trimethylbenzenesulfonylamino)-phenyl]-2,4,6-trimethylbenzenesulfonamide;
N-[4-(4-methoxy-phenoxy)-2-(4-nitrobenzenesulfonylamino)-phenyl]-4-nitrobenzenesulfonamide;
N-[4-(5-hydroxy-pentyloxy)-2-(4-toluenesulfonylamino)-phenyl]-4-toluenesulfonamide;
N-[4-(4-fluoro-phenoxy)-2-(4-toluenesulfonylamino)-phenyl]-4-toluenesulfonamide;
N-[4-(naphthalene-2-yloxy)-2-(4-toluenesulfonylamino)-phenyl]-4-toluenesulfonamide;
N-[4-methoxy-2-(4-toluenesulfonylamino)-phenyl]-4-toluenesulfonamide
N-[4-(4-chloro-phenoxy)-2-(4-toluenesulfonylamino)-phenyl]-4-toluenesulfonamide;
N-[4-(3-carboxy-phenoxy)-2-(4-toluenesulfonylamino)-phenyl]-4-toluenesulfonamide;
{3-[3,4-bis(4-toluenesulfonylamino)-phenoxy]-phenyl}-acetic acid;
N-[4-(3-carboxy-phenoxy)-2-(4-toluenesulfonylamino)-phenyl]-4-toluenesulfonamide;
{4-[3,4-bis(4-toluenesulfonylamino)-phenoxy]-phenyl}-acetic acid;
N-[4-(3-hydroxypropoxy)-2-(4-toluenesulfonylamino)-phenyl]-4-toluenesulfonamide;
N-[4-allyloxy-2-(4-toluenesulfonylamino)-phenyl]-4-toluenesulfonamide;
N-[4-(8-hydroxy-octyloxy)-2-(4-toluenesulfonylamino)-phenyl]-4-toluenesulfonamide;
5-[3,4-bis-(4-toluenesulfonylamino)-phenoxy]-pentyl acetate;
N-[4-(4-chloro-phenylsulfanyl)-2-(4-toluenesulfonylamino)-phenyl]-4-toluenesulfonamide;
N-[4-(4-methoxy-phenylsulfanyl)-2-(4-toluenesulfonylamino)-phenyl]-4-toluenesulfonamide;
N-[4-butylsulfanyl-2-(4-toluenesulfonylamino)-phenyl]-4-toluenesulfonamide;
N-[3-allyl-4-(4-methoxy-benzyloxy)-2-(4-toluenesulfonylamino)-phenyl]-4-toluenesulfonamide;
N-[4-(4-chloro-benzyloxy)-2-(4-toluenesulfonylamino)-phenyl]-4-toluenesulfonamide;
N-[4-(3-chloro-benzyloxy)-2-(4-toluenesulfonylamino)-phenyl]-4-toluenesulfonamide;
N-[4-(2-chloro-benzyloxy)-2-(4-toluenesulfonylamino)-phenyl]-4-toluenesulfonamide;
N-[4-(4-methoxy-benzyloxy)-2-(4-toluenesulfonylamino)-phenyl]-4-toluenesulfonamide;
N-[4-cyclohexylmethoxy-2-(4-toluenesulfonylamino)-phenyl]-4-toluenesulfonamide;
N-[4-(2-chloro-benzylsulfanyl)-2-(4-toluenesulfonylamino)-phenyl]-4-toluenesulfonamide;
N-[4-(pyrazol-1-yl)-2-(4-toluenesulfonylamino)-phenyl]-4-toluenesulfonamide;
N-[4-(benzimidazol-1-yl)-2-(4-toluenesulfonylamino)-phenyl]-4-toluenesulfonamide;
N-[4-dimethylamino-2-(4-toluenesulfonylamino)-phenyl]-4-toluenesulfonamide;
N-[4-(imidazol-1-yl)-2-(4-toluenesulfonylamino)-phenyl]-4-toluenesulfonamide;
N-{2-(4-toluenesulfonylamino)-4-[4-(toluene-4-sulfonyl)-piperazin-1-yl]-phenyl}-4-toluenesulfonamide;
N-[4-(morpholin-4-yl)-2-(4-toluenesulfonylamino)-phenyl]-4-toluenesulfonamide;
N-[4-(4-chloro-phenyl)-2-(4-toluenesulfonylamino)-phenyl]-4-toluenesulfonamide;
N-[3-methoxy-2-(4-toluenesulfonylamino)-phenyl]-4-toluenesulfonamide;
N-[2-(4-methoxy-phenyl)-ethyl]-3,4-bis-(4-toluenesulfonylamino)-benzamide;
N-t-butyl-3,4-bis-(4-toluenesulfonylamino)-benzamide;
N-pyridin-3-yl-3,4-bis-(4-toluenesulfonylamino)-benzamide;
N-phenyl-3,4-bis-(4-toluenesulfonylamino)-benzamide;
N-(3-hydroxy-2,2-dimethyl-propyl)-3,4-bis-(4-toluenesulfonylamino)-benzamide;
N-naphthalen-1-ylmethyl-3,4-bis-(4-toluenesulfonylamino)-benzamide;
2-phenyl-N-[3,4-bis-(4-toluenesulfonylamino)-phenyl]-acetamide;
N-[3,4-bis-(4-toluenesulfonylamino)-phenyl]-thiophene-2-sulfonamide;
3,5-dimethyl-N-[3,4-bis-(4-toluenesulfonylamino)-phenyl]-isoxazole-4-sulfonamide;
3,4-bis-(4-toluenesulfonylamino)-benzoic acid;
N-[4-hydroxymethyl-2-(4-toluenesulfonylamino-phenyl]-4-toluenesulfonamide;
N-[2-methanesulfonylamino-4-(4-methoxy-phenoxy)-phenyl]-4-toluenesulfonamide;
3,5-dimethyl-N-[5-(4-methoxy-phenoxy)-2-(4-toluenesulfonylamino)-phenyl]-isoxazole-4-sulfonamide;
N-[5-(4-methoxy-phenoxy)-2-(4-toluenesulfonylamino)-phenyl]-1-methyl-1H-imidazole-4-sulfonamide;
N-[2-methanesulfonylamino-5-(4-methoxy-phenoxy)-phenyl]-4-toluenesulfonamide;
N-{5-[4-(4-methoxy-phenoxy)-2-(4-toluenesulfonylamino)-phenylsulfamoyl]-4-methyl-thiazol-2-yl}-acetamide;
3,5-dimethyl-N-[4-(4-methoxy-phenoxy)-2-(4-toluenesulfonylamino)-phenyl]-isoxazole-4-sulfonamide;
N-[4-(4-methoxy-phenoxy)-2-(4-toluenesulfonylamino)-phenyl]-1-methyl-1H-imidazole-4-sulfonamide;
5-bromo-6-chloro-N-[4-(4-methoxy-phenoxy)-2-(4-toluenesulfonylamino)-phenyl]-pyridine-3-sulfonamide;
7-chloro-N-[4-(4-methoxy-phenoxy)-2-(4-toluenesulfonylamino)-phenyl]-benzo[1,2,5]oxadiazole-4-sulfonamide;
5-isoxazol-3-yl-N-[4-(4-methoxy-phenoxy)-2-(4-toluenesulfonylamino)-phenyl]-thiophene-2-sulfonamide;
methyl 4-[4-(4-methoxy-phenoxy)-2-(4-toluenesulfonylamino)-phenylsulfamoyl]-1,2,5-trimethyl-1H-pyrrole-3-carboxylate;
4-butyl-N-[4-(4-methoxy-phenoxy)-2-(4-toluenesulfonylamino)-phenyl]-benzenesulfonamide;
N-[5-(4-methoxy-phenoxy)-2-(2-naphthalen-1-yl-ethanesulfonylamino)-phenyl]-4-toluenesulfonamide;
4-methanesulfonyl-N-[4-(4-methoxy-phenoxy)-2-(4-toluenesulfonylamino)-phenyl]-benzenesulfonamide;
3-methoxy-N-[4-(4-methoxy-phenoxy)-2-(4-toluenesulfonylamino)-phenyl]-benzenesulfonamide;
N-[4-(4-methoxy-phenoxy)-2-(4-toluenesulfonylamino)-phenyl]-5-bromo-thiophene-2-sulfonamide;
N-[4-(4-methoxy-phenoxy)-2-(4-toluenesulfonylamino)-phenyl]-isoquinoline-5-sulfonamide;
methyl 4-[5-(4-methoxy-phenoxy)-2-(4-toluenesulfonylamino)-phenylsulfamoyl]-1,2,5-trimethyl-1H-pyrrole-3-carboxylate;
N-[4-(4-methoxy-phenoxy)-2-(2-naphthalen-1-yl-ethanesulfonylamino)-phenyl]-4-toluenesulfonamide;
4-methanesulfonyl-N-[5-(4-methoxy-phenoxy)-2-(4-toluenesulfonylamino)-phenyl]-benzenesulfonamide;
5-bromo-N-[5-(4-methoxy-phenoxy)-2-(4-toluenesulfonylamino)-phenyl]-thiophene-2-sulfonamide;
2-methoxy-N-[5-(4-methoxy-phenoxy)-2-(4-toluenesulfonylamino)-phenyl]-4-methyl-benzenesulfonamide;
N-[4-(benzyloxyimino-phenyl-methyl)-2-(4-toluenesulfonylamino)-phenyl]-4-toluenesulfonamide;
N-[4-(hydroxyimino-phenyl-methyl)-2-(4-toluenesulfonylamino)-phenyl]-4-toluenesulfonamide;
N-[4-(methoxyimino-phenyl-methyl)-2-(4-toluenesulfonylamino)-phenyl]-4-toluenesulfonamide;
N-[5-(2-chloro-benzylsulfanyl)-2-methanesulfonylamino-phenyl]-4-toluenesulfonamide;
N-[4-(2-chloro-benzylsulfanyl)-2-(4-toluenesulfonylamino)-phenyl]-4-toluenesulfonamide;
N-[4-(2-chloro-benzylsulfanyl)-2-methanesulfonylamino-phenyl]-4-toluenesulfonamide;
N-[5-amino-4-(4-chloro-phenylsulfanyl)-2-(4-toluenesulfonylamino)-phenyl]-4-toluenesulfonamide;
N-[2-(4-chloro-phenylsulfanyl)-4,5-bis-(4-toluenesulfonylamino)-phenyl]-acetamide;
methyl 2-(4-methoxy-phenoxy)-4,5-bis-(4-toluenesulfonylamino)-benzoate;
2-(4-methoxy-phenoxy)-4,5-bis-(4-toluenesulfonylamino)-benzoic acid;
N-[4-(4-methoxy-phenoxy)-5-methyl-2-(4-toluenesulfonylamino)-phenyl]-4-toluenesulfonamide;
N-[4,5-dibromo-2-(4-toluenesulfonylamino)-phenyl]-4-toluenesulfonamide;
N-[4-(4-methoxy-phenoxy)-2-(4-toluenesulfonylamino)-5-trifluoromethyl-phenyl]-4-toluenesulfonamide;
N-[4-(2-chloro-benzylsulfanyl)-5-fluoro-2-(4-toluenesulfonylamino)-phenyl]-4-toluenesulfonamide;
N-[4-(2-chloro-phenylmethanesulfonyl)-5-fluoro-2-(4-toluenesulfonylamino)-phenyl]-4-toluenesulfonamide;
diethyl-5-(4-methoxy-phenoxy)-2-(4-toluenesulfonylamino)-phenyl phosphate;
[5-(4-methoxy phenoxy)-2-(4-toluenesulfonylamino)-phenyl]-monophosphate;
4-(4-methoxy-phenoxymethyl)-2-(4-toluenesulfonyloxy)-phenyl 4-toluenesulfonate;
N-[4-(4-chloro-phenylsulfanyl)-5-nitro-2-(4-toluamido)-phenyl]-4-toluamide;
N-[4-(4-chloro-phenylsulfanyl)-2-(4-toluoylamino)-phenyl]-4-toluamide;
N-[4-(4-chloro-phenylsulfanyl)-5-nitro-2-(4-toluenesulfonyl-methyl-amino)-phenyl]-N-methyl-4-toluenesulfonamide;
N-[2-amino-4-(4-chloro-phenylsulfanyl)-5-nitro-phenyl]-4-toluenesulfonamide;
N-[2-amino-4-(4-chloro-phenylsulfanyl)-5-nitro-phenyl]-4-toluamide;
N-[2-hydroxy-4-(4-methoxy-phenoxy)-phenyl]-4-toluenesulfonamide;
N-[2-methoxy-4-(4-methoxy-phenoxy)-phenyl]-4-toluenesulfonamide;
N-[2-methoxy-4-(4-methoxy-phenoxy)-phenyl]-N-methyl-4-toluenesulfonamide;
N-[2-(2-hydroxy-ethoxy)-4-(4-methoxy-phenoxy)-phenyl]-4-toluenesulfonamide;
N-[2-(3-hydroxy-propoxy)-4-(4-methoxy-phenoxy)-phenyl]-4-toluenesulfonamide;
N-[2-(2,3-dihydroxy-propoxy)-4-(4-methoxy-phenoxy)-phenyl]-4-toluenesulfonamide;
[5-(4-methoxy-phenoxy)-2-(4-toluenesulfonylamino)-phenoxy]-acetic acid;
N-[2-methoxy-5-(4-methoxy-phenoxymethyl)-phenyl]-4-toluenesulfonamide;
methyl 5-(4-methoxy-phenoxy)-2-(4-toluenesulfonylamino)-benzoate;
5-(4-methoxy-phenoxy)-2-(4-toluenesulfonylamino)-benzoic acid;
N-[4-(4-chloro-phenylsulfanyl)-3-nitro-phenyl]-4-toluenesulfonamide;
N-[3-(4-chloro-phenylsulfanyl)-4-nitro-phenyl]-4-toluenesulfonamide;
N-[2-amino-5-(4-methoxy-phenoxy)-phenyl]-4-toluenesulfonamide;
6-cyano-N-[4-(4-methoxy-phenoxy)-2-(4-toluenesulfonylamino)-phenyl]-nicotinamide;
N-[4-(4-methoxy-phenoxy)-2-(4-toluenesulfonylamino)-phenyl]-6-trifluoromethyl-nicotinamide;
N-[2-(4-cyano-benzylamino)-5-(4-methoxy-phenoxy)-phenyl]-4-toluenesulfonamide;
N-(4-{[4-(4-methoxy-phenoxy)-2-(4-toluenesulfonylamino)-phenylamino]-methyl}-phenyl)-acetamide;
N-[2-[(benzofuran-2-ylmethyl)-amino]-5-(4-methoxy-phenoxy)-phenyl]-4-toluenesulfonamide;
N-{5-(4-methoxy-phenoxy)-2-[(quinolin-2-ylmethyl)-amino]-phenyl}-4-toluenesulfonamide;
N-[5-(4-methoxy-phenoxy)-2-(4-toluenesulfonylamino)-phenyl]-5-methyl-pyrazine-2-carboxamide;
N-[2-amino-5-(4-methoxy-phenoxy)-benzyl]-4-toluenesulfonamide;
N-[2-cyano-4-(4-methoxy-phenoxy)-phenyl]-4-toluenesulfonamide;
5-(4-methoxy-phenoxy)-2-(4-toluenesulfonylamino)-benzamide;
N-[2-amino-4-(4-methoxy-phenoxy)-phenyl]-4-toluenesulfonamide;
N-[4-(4-methoxy-phenoxy)-2-(4-toluenesulfonylamino-methyl)-phenyl]-4-toluenesulfonamide;
N-[2-aminomethyl-4-(4-methoxy-phenoxy)-phenyl]-4-toluenesulfonamide;
N-[3-[benzoyl-(4-toluenesulfonyl)-amino]-4-(4-toluenesulfonylamino)-phenyl]-benzamide;
N-[5-(4-methoxy-phenoxy)-2-(4-toluenesulfonylamino)-benzyl]-4-toluamide;
N-[5-(4-methoxy-phenoxy)-2-(4-toluenesulfonylamino)-phenyl]-4-toluamide;
N-[4-(4-methoxy-phenoxy)-2-(4-toluenesulfonylamino)-phenyl]-4-toluamide;
N-[4-(4-methoxy-phenoxy)-2-(4-toluenesulfonylamino)-phenyl]-2-phenyl-acetamide;
N-[2-amino-5-(4-methoxy-phenoxy)-phenyl]-N-methyl-4-toluenesulfonamide;
N-{4-(4-methoxy-phenoxy)-2-[(quinolin-2-ylmethylene)-amino]-phenyl}-4-toluenesulfonamide;
4-(4-methoxy-phenoxymethyl)-2-(4-toluenesulfonylamino)-benzamide;
N-[2-aminomethyl-5-(4-methoxy-phenoxymethyl)-phenyl]-4-toluenesulfonamide;
N-[2-(methanesulfonylamino-methyl)-5-(4-methoxy-phenoxymethyl)-phenyl]-6-methyl-naphthalene-2-sulfonamide;
N-(4-morpholin-4-yl-5-nitro-2-(4-toluoylamino)-phenyl)-4-toluamide;
N-[5-(4-methoxy-phenoxy)-2-(4-toluenesulfonylamino)-phenyl]-butyramide;
N-[4-(cyclohexylmethyl-amino)-5-nitro-2-(4-toluoylamino)-phenyl]-4-toluamide;
N-{4-[(naphthalen-1-ylmethyl)-amino]-5-nitro-2-(4-toluoylamino)-phenyl}-4-toluamide;
N-[2-amino-4-(4-methoxy-phenoxy)-5-nitro-phenyl]-4-toluenesulfonamide;
N-[4-(4-methoxy-phenoxy)-2-(4-toluenesulfonyl-methyl-amino)-phenyl]-4-toluenesulfonamide;
N-[2-amino-4-(4-methoxy-phenoxy)-5-trifluoromethyl-phenyl]-4-toluenesulfonamide;
N-[4-(4-methoxy-phenoxymethyl)-2-(4-toluenesulfonylamino)-benzyl]-4-toluamide;
N-[2-amino-5-(4-methoxy-phenoxy)-4-trifluoromethyl-phenyl]-4-toluenesulfonamide;
N-[4-(4-methoxy-phenoxy)-2-(4-methyl-benzylamino)-phenyl]-4-toluenesulfonamide;
N-{4-(4-methoxy-phenoxy)-2-[3-(4-toluenesulfonyl)-ureido]-phenyl}-4-toluenesulfonamide;
N-[5-(4-methoxy-phenoxy)-2-(4-methyl-benzylamino)-phenyl]-4-toluenesulfonamide;
N-[2-amino-5-(2-chloro-benzylsulfanyl)-phenyl]-4-toluenesulfonamide;
N-[2-amino-4-(2-chloro-benzylsulfanyl)-5-nitro-phenyl]-7-chloro-benzo[1,2,5]oxadiazole-4-sulfonamide;
N-(4-ethyl-phenyl)-4-methoxy-2-(4-toluenesulfonylamino)-benzenesulfonamide;
N-[4-(2-chloro-phenylmethanesulfonyl)-2-(4-toluenesulfonylamino)-phenyl]-4-toluenesulfonamide;
6-(4-methoxy-phenoxy)-1-(4-toluenesulfonyl)-1,3-dihydro-benzoimidazol-2-one;
5-(4-methoxy-phenoxy)-1-(4-toluenesulfonyl)-1,3-dihydro-benzoimidazol-2-one;
5-(4-methoxy-phenoxy)-1,3-bis-(4-toluenesulfonyl)-1,3-dihydro-benzoimidazol-2-one;
5-(4-methoxy-phenoxy)-1,3-dihydro-benzo[1,2,5]thiadiazole 2,2-dioxide;
5-(4-methoxy-phenoxy)-1,3-dihydro-benzoimidazol-2-one;
5-(4-methoxy-phenoxy)-1-(4-toluenesulfonyl)-1,3-dihydro-benzo[1,2,5]thiadiazole 2,2-dioxide; and mixtures thereof.

3. The compound of claim 1 wherein the compound has the structure of Formula I.

4. The compound of claim 3 wherein
R₁ and R₂ are p-(C₆H₄)CH₃.

5. The compound of claim 4 wherein R₄ is selected from the group consisting of p-(C₆H₄)OCH₃, o-(C₆H₄)OCH₃, m-(C₆H₄)OCH₃, p-(C₆H₄)OH, p-(C₆H₄)Cl, o-(C₆H₄)Cl, m-(C₆H₄)Cl, p-(C₆H₄)F, p-(C₆H₄)CH₃, o-(C₆H₄)CH₃, m-(C₆H₄)CH₃, 3,5-(C₆H₃)(CH₃)₂, 2,6-(C₆H₃)(CH₃)₂, o-(C₆H₄)[CH(CH₃)₂], and C₆H₅.

6. The compound of claim 5 wherein Y₁ is not present.

7. The compound of claim 6 wherein X₁ is S.

8. The compound of claim 1 wherein the compound has the structure of Formula II.

9. The compound of claim 8 wherein wherein R₄ is selected from the group consisting of p-(C₆H₄)CH₃, p-(C₆H₄)Cl, p-(C₆H₄)[C(CH₃)]₃, p-(C₆H₄)NO₂, 2-naphthyl and 1-naphthyl.

10. The compound of claim 9 wherein R₅ is selected from the group consisting of p-(C₆H₄)CH₃, p-(C₆H₄)Cl, 1-naphthyl, 2-naphthyl, p-(C₆H₄)[C(CH₃)]₃, and p-(C₆H₄)NO₂.

11. The compound of claim 10 wherein R₆ is selected from the group consisting of p-(C₆H₄)OCH₃, p-(C₆H₄)Cl, m-(C₆H₄)CO₂H, m-(C₆H₄)CH₂CO₂H, p-(C₆H₄)CO₂H, p-(C₆H₄)CH₂CO₂H, and o-(C₆H₄)Cl.

12. The compound of claim 11 wherein R₇ is H.

13. The compound of claim 12 wherein X₂ is O or S.

14. The compound of claim 13 wherein Y₂ is CH₂ or is not present.

15. The compound of claim 1 wherein the compound has the structure of Formula III.

16. The compound of claim 15 wherein R₁₀ is o-(C₆H₄)Cl and R₁₁ is F.

17. The compound of claim 16 wherein X₃ is S or SO₂ and Y₃ is CH₂.

18. The compound of claim 1 wherein the compound has the structure of Formula IV.

19. The compound of claim 1 wherein the compound has the structure of Formula V.

20. A method of inhibiting a protein tyrosine kinase by administering a subject at least one compound of claim 1.

21. The method of claim 20 further comprising the step of the binding of the compound to said protein tyrosine kinase.

22. The method of claim 20 wherein the protein tyrosine kinase is selected from the group consisting of Src, Fyn, Yes, Lyn, Lck, Blk, Hck, and Fgr.

23. The method of claim 20 wherein the subject is a mammal.

24. The method of claim 21 wherein the mammal is a human.

25. The method of claim 20 wherein the administering is parenteral.

26. The method of claim 25 wherein the parenteral administration is intravenous, intramuscular, subcutaneous, intraperitoneal, intraarterial, intrathecal or transdermal.

27. The method of claim 25 wherein the administering is alimentary.

28. The method of claim 27 wherein the alimentary administration is oral, rectal, sublingual, or buccal.

29. The method of claim 20 wherein the administration is topical.

30. The method of claim 20 wherein the administration is by inhalation.

31. A pharmaceutical composition comprising a carrier and at least one compound of claim 1.
